(19) United States
(12) Patent Application Publication
(10) Pub. No.: US 2026/0092094 A1
(43) Pub. Date: Apr. 2, 2026

(54) ENGINEERED CYTOKINES AND USES THEREOF

(71) Applicant: Peptone Ltd., London (GB)

(72) Inventors: Louie HENDERSON, London (GB); Oliver DUTTON, London (GB); Falk HOFFMANN, Soest (DE); Sandro BOTTARO, Milanese (IT); Istvan REDL, London (GB); Angela Rita ELIA, Bellinzona (CH); Carlo FISICARO, Cadro (CH); Benjamin OWENS, Buckinghamshire (GB); Patrick KUNZ, Bellinzona (CH); Matthew LAKINS, London (GB); Patrik FOERCH, London (GB); David LOWE, London (GB); Kamil TAMIOLA, Carasso (CH)

(21) Appl. No.: 19/481,848

(22) PCT Filed: May 3, 2024
(86) PCT No.: PCT/IB2024/054340

§ 371 Date: Nov. 5, 2025

Related U.S. Application Data
(60) Provisional application No. 63/500,104, filed on May 4, 2023.

(51) Int. Cl. C07K 14/54

(52) U.S. Cl. CPC C07K 14/54

(57) ABSTRACT

Disclosed herein are engineered cytokines, compositions and uses thereof, and in silico methods for engineering cytokines generated from artificial neural network machine learning protocols and/or molecular dynamics simulations. Also disclosed herein are poly peptide structures designed using AI methods with modulated stability, potency and developability. Also disclosed herein are compositions containing engineered cytokine for pharmaceutical use, as well as methods of treating a subject by administering to the subject compositions containing the same.

### CROSS REFERENCE TO RELATED APPLICATIONS

This application claims priority to U.S. Application No. 63/500,104, filed on 4 May 2023, the disclosure of which is incorporated herein by reference in its entirety.

### BACKGROUND

While techniques such as X-ray crystallography can be used to elucidate a polypeptide structure, such techniques are hampered in poorly expressed or poorly folded proteins. Numerous drugs are commercially available for targeting cancer and immune regulatory conditions. However, the stability; potency, and developability, are all challenges for biologic product development. Thus, there is a need for improved therapeutics to target such diseases and conditions.

### SUMMARY

Provided herein are compositions comprising: an engineered IL-21 cytokine or a functional fragment thereof, that is a variant of a wild type IL-21 cytokine having amino acid residues of SEQ ID NO: 1, wherein the engineered IL-21 cytokine or a functional fragment thereof includes at least one amino acid substitution in a region including amino acid residues 30 to 135 of SEQ ID NO: 1, wherein the at least one amino acid substitution provides for an increased stability of the engineered IL-21 cytokine compared to the wild type IL-21 cytokine, and wherein the increased stability is characterized by an increased thermal stability of the engineered IL-21 cytokine compared to that of the wild type IL-21 cytokine when measured in a 75 ug/mL sample using a differential scanning fluorimetry (DSF) spectroscopic method to detect thermal melting measurements by performing a thermal ramp with a 1 degree Celsius/minute heating rate from 20 degrees Celsius to 95 degrees Celsius and measuring backscattered light intensity to detect heat induced aggregation.

Provided herein are compositions comprising an engineered cytokine that is a variant of a wild type cytokine including a tertiary structure with 4 alpha helices, wherein the engineered cytokine includes at least one amino acid substitution in a non-alpha helical coil region compared to the wild type cytokine that provides for an increased stability of the engineered cytokine compared to the wild type cytokine, wherein the increased stability is characterized by an increased thermal stability of the engineered cytokine compared to that of the wild type cytokine when measured in a 75 ug/mL sample using a differential scanning fluorimetry (DSF) spectroscopic method to detect thermal melting measurements by performing a thermal ramp with a 1 degree Celsius/minute heating rate from 20 degrees Celsius to 95 degrees Celsius and measuring backscattered light intensity to detect heat induced aggregation.

Provided herein are compositions comprising: an engineered cytokine that is a variant of a wild type cytokine including a disordered region, wherein the engineered cytokine includes at least one amino acid substitution in the disordered region, wherein the at least one amino acid substitution provides for increased stability of the engineered cytokine compared to the wild type cytokine, and wherein the increased stability is characterized by an increased thermal stability of the engineered cytokine compared to the wild type cytokine when measured in a 75 ug/mL sample using a differential scanning fluorimetry (DSF) spectroscopic method to detect thermal melting measurements by performing a thermal ramp with a 1 degree Celsius/minute heating rate from 20 degrees Celsius to 95 degrees Celsius and measuring backscattered light intensity to detect heat induced aggregation.

Provided herein are pharmaceutical compositions comprising the compositions disclosed herein.

Provided herein are methods of inducing cell death, the methods comprising contacting a cell with the composition or pharmaceutical composition disclosed herein.

Provided herein are methods of reducing cancer cell growth, the methods comprising contacting a cell with the composition or pharmaceutical composition disclosed herein.

Provided herein are methods of modulating an immune response in a subject, the methods comprising administering to the subject an effective amount of the composition or pharmaceutical composition disclosed herein.

Provided herein are methods of treating proliferative diseases or fibrotic disorders in a subject, the methods comprising administering to the subject an effective amount of the composition or pharmaceutical composition disclosed herein.

Provided herein are methods of treating cancer in a subject in need thereof, the methods comprising administering to the subject an effective amount of the composition or pharmaceutical composition disclosed herein.

Provided herein are polynucleotides encoding the engineered IL-21 cytokines or a functional fragment thereof described herein or the engineered cytokines described herein.

Provided herein are vectors including the polynucleotides encoding the engineered IL-21 cytokines or a functional fragment thereof described herein or the engineered cytokines described herein.

Provided herein are host cells comprising the vectors disclosed herein.

Provided herein are oncolytic viruses including an exogenous nucleic acid that codes for the engineered IL-21 cytokines or a functional fragment thereof described herein or the engineered cytokines described herein.

### BRIEF DESCRIPTION OF THE DRAWINGS

Features of exemplary embodiments are set forth with particularity in the appended claims. A better understanding of the features and advantages will be obtained by reference to the following detailed description that sets forth illustrative embodiments, in which the principles of the disclosed systems and methods are utilized, and the accompanying drawings of which:

FIG. 1 depicts an exemplary workflow for generating optimal mutant cytokines consistent with embodiments described herein.

FIG. 2 illustrates cytokine drug engineering, including fusion protein comprising cytokine, antibody-cytokine immune complex, cytokine PEGylation, and cytokine mutagenesis.

FIG. 3 illustrates the structures of ordered IL-21, disordered IL-21, and IL-2 in the presence of an IL-21 receptor.

FIG. 4A-FIG. 4B shows secondary structure analysis of human IL-21 in different conformations. FIG. 4A is a plot showing disorder in regions of the human IL-21 protein. FIG. 4B shows alternative conformations in IL-21, including IL-21 in disordered state (minor) and folded state (major) performed by biased molecular dynamics.

FIG. 5 illustrates the region in IL-21 that forms helix C after the disorder-to-order transition.

FIG. 6 illustrates AI driven mutant design of mutations that destabilizes the disordered state of IL-21 (left) and forces the protein to populate ordered and compact conformations (right).

FIG. 7 illustrates the results of mutation prediction for 250,000 triplet mutant populations of IL-21. X axis of the plot shows the number of triplet mutant populations. Y axis of the plot shows the fitness score for every triplet mutant population as predicted by AI. The IL-21 sequence is listed on the top of the plot.

FIG. 8 is a plot showing the predicted folding stability of two exemplary IL-21 variants as compared to the wild type as evaluated by the ADOPT order score for each amino acid residue position predicted by AI.

FIG. 9 are graphs and plots showing the results of integrative modelling and mutant prediction for IL-21 using the software ProteinPrime. The top left graph is the amino acid sequence alignment of IL-21 wild type and mutants. The bottom left plots show the prediction of the disordered region for IL-21 wild type and mutants. Left graph shows the structure of an exemplary IL-21.

FIG. 10A-FIG. 10B show the comparisons of the productions of different IL-21 variants. FIG. 10A is a table showing the number of mutations and relative yield of different IL-21 variants as compared to the wild type. FIG. 10B shows the SDS-PAGE analysis of purified IL-21 variants expressed in HEK-293 cells. The bands from left to right represent proteins in different sizes in the loading dye and the IL-21 variants shown in TABLE 1.

FIG. 11A-FIG. 11D show size exclusion chromatography (SEC) chromatogram traces and intact mass analysis profiles of IL-21 wild type and variants. FIG. 11A shows an SEC chromatogram and the corresponding mass spectrometry trace for an exemplary IL-21 variant.

FIG. 11B-FIG. 11D are SEC chromatograms of IL-21 wild type and 3 variants. Each plot shows the UV absorbance at 280 nm (Y axis) versus elution time (X axis).

FIG. 12A-FIG. 12C shows pepsin digestion fragments of IL-21. FIG. 12A-FIG. 12C are graphs mapping the fragments of IL-21 wild type and variants digested by pepsin.

FIG. 13 are structures showing the rigidity of IL-21 wild type and two variants as predicted by hydrogen-deuterium exchange mass spectrometry experiments. The darkest regions are the most rigid regions with a slow proton-deuterium exchange rate. The light gray regions are the flexible regions with a fast proton-deuterium exchange rate.

FIG. 14A-FIG. 14B demonstrates structural rigidity from mutations introduced by Oppenheimer's generative AI increases Il-21 compactness and thermostability. FIG. 14A is a graph showing the particle size of IL-21 wild type and mutants as determined by dynamic light scattering (DLS). FIG. 14B is a graph comparing the melting temperature (Tm) for IL-21 wild type and exemplary variants.

FIG. 15A-FIG. 15B are results showing the expression of IL-21R in M116 cell line as determined by flow-cytometry. FIG. 15A are graphs showing sorting of M116 cells expressing a control antibody IgG1PE (top) and IL-21 receptor (bottom) FIG. 15B are plots of cells on a histogram with the number of cells normalized to the mode on the y-axis and IL-21 receptor antibody on the x-axis.

FIG. 16A-FIG. 16B are plots and bar charts showing the viability of MC116 cells in the presence of AI designed IL-21 variants. FIG. 16A are plots showing the viability of MC116 cells with increasing concentration of IL-21 WT and exemplary variants. FIG. 16B are bar charts showing the average percentage of MC116 viability in the presence of 25 ng/ml IL-21 WT and variants.

FIG. 17A-FIG. 17D are plots and bar charts showing STAT3 phosphorylation triggered by IL-21 in MC116 cell lines as determined by flow-cytometry. FIG. 17A are plots of cells on histograms with the number of cells normalized to the mode on the y-axis and anti-STAT3 phospho (Tyr705) antibody on the x-axis. FIG. 17B-FIG. 17D are bar charts showing the fold induction in the presence of 6.25 ng/ml (FIG. 17B), 25 ng/ml (FIG. 17C), and 100 ng/ml (FIG. 17D).

FIG. 18A-FIG. 18B are results demonstrating AI designed variants trigger STAT3 in MC116 cell lines. FIG. 18A are plots of histograms with M116 cellular events untreated, treated with polypeptides 1, 41, and 78, respectively. In each plot, the dots on the left represents live cells, the dots on the right represents cells undergoing early apoptosis. FIG. 18B are bar charts showing the percentage of cells undergoing apoptosis and necrosis when treated with polypeptide 1, polypeptide 41, and polypeptide 78.

FIG. 19 are structures of IL-2, IL-4, IL-7, IL-9, and IL-15.

FIG. 20A-FIG. 20J are plots and bar charts showing STAT3 phosphorylation in MC116 cell line induced by IL-21 wild type and variants at low concentrations. FIG. 20A-FIG. 20C are plots of cells on histograms with the number of MC116 cells in the presence of polypeptide 1 (FIG. 20A), Polypeptide 41 (FIG. 20B), and polypeptide 78 (FIG. 20C) at different concentrations normalized to the mode on the y-axis and anti-STAT3 phospho (Tyr705) antibody on the x-axis. FIG. 20D are plots of cells on histograms with the number of MC116 cells in the presence of 0.1 ng/ml Polypeptide 1, Polypeptide 41, and Polypeptide 78, respectively, at different concentrations normalized to the mode on the y-axis and anti-STAT3 phospho (Tyr705) antibody on the x-axis. FIG. 20E-FIG. 20G are bar charts showing the percentage of phosphorylated STAT3 in MC116 cells in the presence of IL-21 WT and variants at 0.1 ng/ml (FIG. 20E), 0.5 ng/ml (FIG. 20F), 2.5 ng/ml (FIG. 20G). FIG. 20H-FIG. 20J are bar charts showing the median of total population of phosphorylated STAT3 in MC116 cells in the presence of IL-21 WT (polypeptide 1) and variants at 0.1 ng/ml (FIG. 20H), 0.5 ng/ml (FIG. 20I), 2.5 ng/ml (FIG. 20J).

FIG. 21A-FIG. 21D are schematic illustrations of the cell treatment procedures for evaluating the level of IL-21R (FIG. 21A) cell viability (FIG. 21B), STAT3 phosphorylation (FIG. 21C), and cell apoptosis (FIG. 21D).

FIG. 22A-FIG. 22D are graphs showing serum stability of IL-21 variants, as measured by HEK IL-21 reporter cells (FIG. 22A at T=0 days and FIG. 22B at T=4 days) and STAT3 phosphorylation (FIG. 22C at T=0 days and FIG. 22D at T=4 days) in physiologically relevant MC116 cells.

FIG. 23 shows pharmacokinetic profiles of an IL-21 variant compared to wild type IL-21.

### DETAILED DESCRIPTION

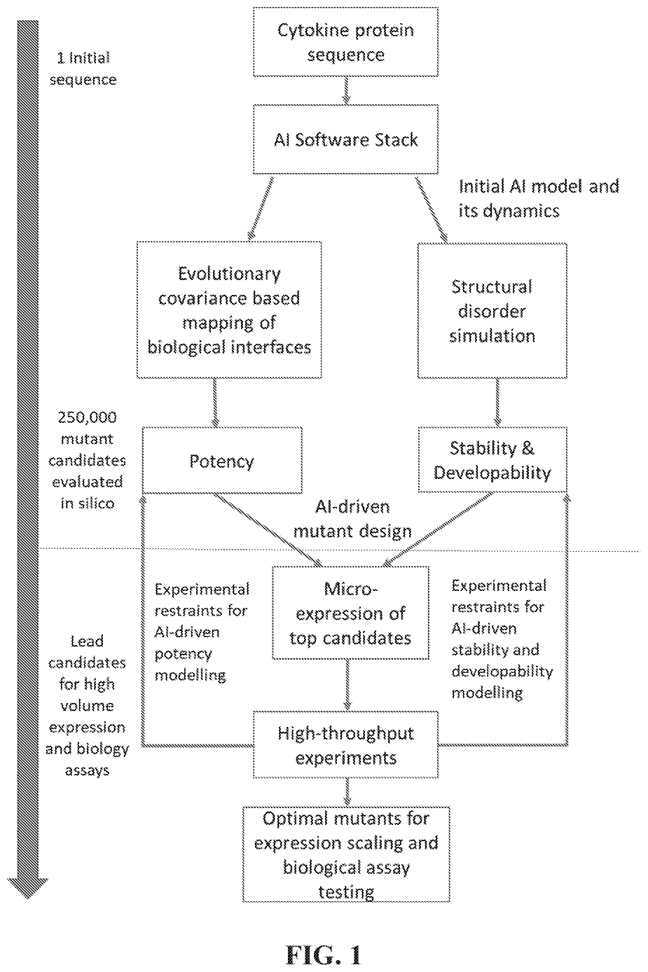

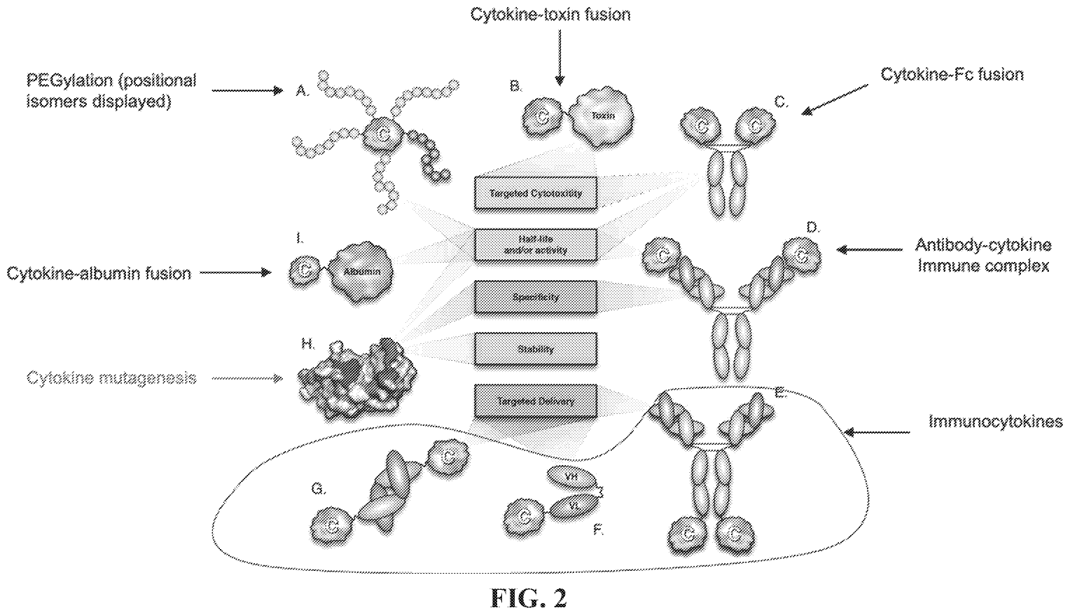

Disclosed herein are engineered cytokines, such as engineered IL-21 cytokines, as well as compositions thereof, and methods of generating and using the same. In some cases, engineered cytokines designed by computer assisted analysis using Artificial Intelligence (AI) assisted protein design is described. Assays to identify functional engineered cytokines were carried out to evaluate potency, stability; and developability. Assays described herein include both in silico techniques, as well as confirmatory empirical lab analyses to arrive at select cytokines with commercially relevant and enhanced features. In some embodiments, select cytokines include engineered cytokines possessing distinct tertiary structures. In some embodiments, the engineered cytokines possess four helical bundles. Engineered cytokines described herein include engineered IL-21 cytokines and related structures. Also described herein are polypeptide structures designed and generated in silico using big data, for example, from molecular dynamics simulations as a function of time. An exemplary workflow for the generation of a variant cytokine protein sequence using methods described herein, including AI driven mutant design is illustrated in FIG. 1. Such data with specific features required by the cytokines of interest are processed using machine learning algorithms as described herein to generate a more fulsome predicted polypeptide structure as compared to using existing methods of structural prediction. By allowing a polypeptide structure to vary as a function of time and sample rare conformations along potential energy wells, the predicted structure can more closely match the dynamics that exist in the polypeptide when present in its natural environment. Using this method, binding surfaces and epitopes that are present through dynamic movement of residues separated by significant sequence space can be accurately mapped, which can allow for generation of robust therapeutics that are able to interact with these epitopes. Furthermore, the AI-driven variant design model described herein lead to a large library of mutant, or variant candidates to be used in high throughput experiments. Lead variant candidates are then identified for stability, developability, and potency profiling.

The section headings used herein are for organizational purposes only and are not to be construed as limiting the subject matter described.

Although various features of the disclosure may be described in the context of a single embodiment, the features can also be provided separately or in any suitable combination. Conversely, although the disclosure may be described herein in the context of separate embodiments for clarity, various aspects and embodiments can be implemented in a single embodiment.

### Definitions

Any methods and materials similar or equivalent to those described herein can be used in the practice for testing of the present disclosure. Accordingly, the terminology used herein is for the purpose of describing particular embodiments only, and is not intended to be limiting.

As used in the specification and claims, the singular form “a,” “an,” and “the” include plural references unless the context clearly dictates otherwise. For example, the term “a polypeptide” includes a plurality of polypeptides, including mixtures thereof.

The term “or” is used disjunctively unless the context specifically refers to a conjunctive use.

Unless specifically stated or obvious from context, as used herein, the term “about” in reference to a number or range of numbers is understood to mean the stated number and numbers +/−10% thereof.

The term “effective amount” or “therapeutically effective amount” refers to an amount that is sufficient to achieve or at least partially achieve the desired effect.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this disclosure. Embodiments defined by each of these transition terms are within the scope of this disclosure.

The terms, “% identical,” “% identity,” “percent identity,” and grammatical equivalents thereof, as used herein, in the context of an amino acid sequence or nucleotide sequence, refer to the percent of residues that are identical between respective positions of two sequences when the two sequences are aligned for maximum sequence identity. The % identity is calculated by dividing the total number of the aligned residues by the number of the residues that are identical between the respective positions of the at least two sequences and multiplying by 100. Generally, computer programs can be employed for such calculations. Illustrative programs that compare and align pairs of sequences, include ALIGN (Myers and Miller, Comput Appl Biosci. 1988 March; 4 (1): 11-7), FASTA (Pearson and Lipman, Proc Natl Acad Sci USA. 1988 April; 85(8): 2444-8: Pearson, Methods Enzymol. 1990; 183:63-98) and gapped BLAST (Altschul et al., Nucleic Acids Res. 1997 Sep. 1; 25(17):3389-40), BLASTP, BLASTN, or GCG (Devereux et al., Nucleic Acids Res. 1984 Jan. 11; 12(1 Pt 1):387-95).

The term “epitope” refers to a portion or structure on a polypeptide that a moiety (e.g., a polypeptide immunoglobulin, antibody; etc.) specifically binds to.

The terms, “bind.” “binding,” “interact,” and “interacting,” as used herein, refer to a non-covalent interaction between macromolecules (e.g., between two polypeptides, between a polypeptide and a nucleic acid; between a polypeptide/guide nucleic acid complex and a target nucleic acid; and the like). While in a state of noncovalent interaction, the macromolecules are said to be “associated” or “interacting” or “binding” (e.g., when a molecule X is said to interact with a molecule Y, it is meant the molecule X binds to molecule Y in a non-covalent manner). Non-limiting examples of non-covalent interactions are ionic bonds, hydrogen bonds, van der Waals interactions, and hydrophobic interactions. Not all components of a binding interaction need be sequence-specific (e.g., contacts with phosphate residues in a DNA backbone), but some portions of a binding interaction may be sequence-specific.

The term “nucleotide,” as used herein, can refer to a base-sugar-phosphate combination. The nucleotide can be composed of three subunit molecules: a nucleobase, a five-carbon sugar (ribose or deoxyribose), and a phosphate. The four nucleobases in DNA can include guanine, adenine, cytosine and thymine: in RNA, uracil can be used in place of thymine. Where DNA sequences are included herein, the corresponding RNA sequences, wherein at least one, two, three, four, five, or all T are replaced with U, are contemplated. A nucleotide can comprise a synthetic nucleotide. A nucleotide can comprise a synthetic nucleotide analog. Nucleotides can be monomeric units of a nucleic acid sequence (e.g., deoxyribonucleic acid (DNA) or ribonucleic acid (RNA)).

The term “pleiotropy” refers to the ability of a cell or cytokine to induce different phenotypic traits that may result in biological differences and/or activities.

The term “paratope” refers to a structure of a moiety (e.g., a polypeptide immunoglobulin, antibody, etc.) that specifically binds to an epitope.

As used herein, the terms “protein,” “peptide,” and “polypeptide” are used interchangeably to designate a series of amino acid residues connected to each other by peptide bonds between the alpha-amino and carboxy groups of adjacent residues. The terms “protein,” “peptide,” and “polypeptide” refer to a polymer of amino acids, including engineered amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein,” “peptide,” and “polypeptide” are used interchangeably herein when referring to a gene product and fragments thereof.

The term “subject” as used herein is interchangeable with the term “patient” and includes human and non-human mammals, including for example: a primate, cow, horse, pig, sheep, goat, dog, cat, or rodent, capable of being colonized by other organisms. A mammal can be any age or at any stage of development (e.g., an adult, teen, child, infant, or a mammal in utero). A mammal can be male or female. A mammal can be a pregnant female. In some embodiments a subject can be a human. In some cases, a human can be more than about: 1 day to about 10 months old, from about 9 months to about 24 months old, from about 1 year to about 8 years old, from about 5 years to about 25 years old, from about 20 years to about 50 years old, from about 1 year to about 130 years old or from about 30 years to about 100 years old. Humans can be more than about: 1, 2, 5, 10, 20, 30, 40, 50, 60, 70, 80, 90, 100, 110, or 120 years of age. Humans can be less than about: 1, 2, 5, 10, 20, 30, 40, 50, 60, 70, 80, 90, 100, 110, 120 or 130 years of age.

As used herein, “substantially pure,” when applied to a molecule, can mean sufficiently homogeneous to appear free of readily detectable impurities by weight as determined by standard methods of analysis, such as thin layer chromatography (TLC), gel electrophoresis and high performance liquid chromatography (HPLC), used by those of skill in the art to assess such purity, or sufficient purity such that further purification would not detectably alter the physical and chemical properties, such as enzymatic and biological activities, of the substance. A substantially chemically pure compound may, however, be a mixture of stereoisomers such as a mixture of enantiomers or diastereomers. In some embodiments, the compositions of the present disclosure are substantially pure or contain one or more substantially pure active ingredients, such as engineered cytokines and/or second therapeutics or pharmaceutically acceptable salts thereof.

The term “supervised learning” refers to a deep learning training method in which the machine is provided data from human sources. The term “unsupervised learning” refers to a deep learning training method in which the machine is not provided data from human sources.

The term “semi-supervised learning” refers to a deep learning training method in which the machine is provided a small amount of data from human sources which is then compared to a larger amount of data from other sources available to the machine.

As used herein, the term “treating” or “treatment” refers to clinical intervention in an attempt to alter the disease course of the individual or subject, or subject in need thereof, or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, decreasing the rate of the progression of a disease or health condition, or amelioration or palliation of the disease state. By preventing progression of a disease or disorder, a treatment can prevent deterioration due to a disorder in an affected or diagnosed subject or subject in need thereof, or a subject suspected of having the disorder, but also a treatment may prevent the onset of the disorder or a symptom of the disorder in a subject at risk for the disorder or suspected of having the disorder.

Although various features of the disclosure may be described in the context of a single embodiment, the features can also be provided separately or in any suitable combination. Conversely, although the disclosure may be described herein in the context of separate embodiments for clarity, various aspects and embodiments can be implemented in a single embodiment.

### Sequences

The nucleic and amino acid sequences listed in the disclosure are shown using standard letter abbreviations for nucleotide bases, and one or three letter codes for amino acids.

### Engineered Cytokine and Chemokine Structures

Cytokines are regulators of the immune system and can be classified according to their function, for example, as pro-inflammatory or anti-inflammatory. Upon binding to the cytokine receptor on a target cell, cytokines can activate enzymes that regulate epigenetic modifications, cytokine synthesis, augmented metabolism, cellular proliferation, and apoptosis. Cytokine pleiotropy refers to the ability to induce different phenotypic traits, resulting in a variety of biological consequences. The ability of cytokines to act on the same receptor indicates their redundancy.

Disclosed herein are engineered cytokines. In some embodiments, the engineered cytokine results in an engineered cytokine. As described herein, an engineered cytokine may be referred to as a “mutant,” or “variant” cytokine. In some embodiments, the cytokine is a T-Natural Killer (TNF) cytokine. In some embodiments, the cytokine is a chemokine. Chemokines include the class of cytokines with functionality or functionalities that attract white blood cells to sites of infection. In some embodiments, the engineered cytokine comprises at least one defined secondary structure. In some embodiments, the engineered cytokine comprises a stable secondary structure. In some embodiments, the engineered cytokine comprises at least one stable secondary structure. In some embodiments, the engineered cytokine comprises at least one transient secondary structure. In some embodiments, the engineered cytokine comprises at least one defined tertiary structure. In some embodiments, the secondary structure comprises an alpha helix. In some embodiments, the secondary structure comprises a beta sheet. In some embodiments described herein are engineered cytokines comprising four helices. In some embodiments, the engineered cytokine is an interleukin. In some embodiments, the interleukin is an interleukin is IL-2, IL-5, IL-7, IL-15, IL-21, or a variant of any of these. In some embodiments, the engineered cytokine is an engineered IL-2 cytokine. In some embodiments, the engineered cytokine is an engineered IL-4 cytokine. In some embodiments, the engineered cytokine is an engineered IL-7 cytokine. In some embodiments, the engineered cytokine is an engineered IL-9 cytokine. In some embodiments, the engineered cytokine is an engineered IL-15 cytokine. In some embodiments, the engineered cytokine is an engineered IL-21 cytokine.

Also disclosed herein are engineered cytokines comprising a disordered region. In some embodiments, the engineered cytokine comprises an amino acid substitution. In some embodiments, the engineered cytokine comprises an amino acid substitution in a disordered region of the cytokine. In some embodiments, the engineered cytokine comprises an amino acid substitution in a region complimentary to a disordered region of the cytokine. In some embodiments, the amino acid substitution provides for increased stability of the engineered cytokine compared to a wild type cytokine. A region of disorder of a cytokine described herein includes regions with no defined secondary or tertiary protein structure. In some embodiments, an engineered cytokine described herein, in comparison with a wild type cytokine, lacks hydrogen bonding exhibited by the wild type cytokine.

In some embodiments, there is provided a composition including: an engineered cytokine that is a variant of a wild type cytokine including a disordered region, wherein the engineered cytokine includes at least one amino acid substitution in the disordered region, wherein the at least one amino acid substitution provides for increased stability of the engineered cytokine compared to the wild type cytokine. In some embodiments, there is provided a composition including an engineered cytokine that is a variant of a wild type cytokine including a tertiary structure with 4 alpha helices, wherein the engineered cytokine includes at least one amino acid substitution in a non-alpha helical coil region compared to the wild type cytokine that provides for an increased stability of the engineered cytokine compared to the wild type cytokine. In some embodiments, the increased stability is characterized by an increased thermal stability of the engineered cytokine compared to the wild type cytokine. In some embodiments, the increased thermal stability is measured in a 75 ug/mL sample using a differential scanning fluorimetry (DSF) spectroscopic method to detect thermal melting measurements. In some embodiments, the thermal melting measurements are obtained by performing a thermal ramp with a 1 degree Celsius/minute heating rate from 20 degrees Celsius to 95 degrees Celsius and measuring backscattered light intensity to detect heat induced aggregation. In some embodiments, the disordered region lacks a stable tertiary structure. In some embodiments, the disordered region includes at least one alpha-helical conformation component. In some embodiments, the engineered cytokine further includes at least one amino acid substitution in a region that is not the disordered region. In some embodiments, the engineered cytokine includes 4 alpha helices. In some embodiments, the wild type cytokine is a human cytokine.

The present disclosure further relates to a composition including: an engineered IL-21 cytokine or a functional fragment thereof, that is a variant of a wild type IL-21 cytokine having amino acid residues of SEQ ID NO: 1, wherein the engineered IL-21 cytokine or a functional fragment thereof includes at least one amino acid substitution in a region including amino acid residues 30 to 135 of SEQ ID NO: 1, wherein the at least one amino acid substitution provides for an increased stability of the engineered IL-21 cytokine compared to the wild type IL-21 cytokine, and wherein the increased stability is characterized by an increased thermal stability of the engineered IL-21 cytokine compared to that of the wild type IL-21 cytokine when measured in a 75 ug/mL sample using a differential scanning fluorimetry (DSF) spectroscopic method to detect thermal melting measurements by performing a thermal ramp with a 1 degree Celsius/minute heating rate from 20 degrees Celsius to 95 degrees Celsius and measuring backscattered light intensity to detect heat induced aggregation.

In some embodiments, the engineered IL-21 cytokine or a functional fragment thereof includes an amino acid sequence having at least 80%, at least 85%, at least 90%, or at least 95% sequence identity to SEQ ID NO: 81. In some embodiments, variant cytokines described herein comprise an engineered cytokine comprising 90%, 91%, 92%, 93%, 94%, 95%, 96%, 97%, 98%, 99%, 99.5%, 99.9%, or 100% sequence identity to a sequence disclosed in SEQ ID NO: 2-SEQ ID NO: 81. In some embodiments, the engineered IL-21 cytokine or a functional fragment thereof includes at least two amino acid substitutions, at least three substitutions, at least four amino acid substitutions, or at least five amino acid substitutions in a region including amino acid residues of SEQ ID NO: 81.

Engineered cytokines described herein include mutant, or variant cytokines. In some embodiments, the engineered IL-21 cytokine or a functional variant thereof includes an amino acid sequence selected from any one of SEQ ID NO: 2 to SEQ ID NO: 160. In some embodiments, variant cytokines described herein comprise an engineered cytokine comprising 90%, 91%, 92%, 93%, 94%, 95%, 96%, 97%, 98%, 99%, 99.5%, 99.9%, or 100% sequence identity to a sequence disclosed in SEQ ID NO: 2-SEQ ID NO: 160. In some embodiments, an engineered cytokine disclosed herein comprises a sequence having at least 80%, 85%, 90%, 95%, or more sequence identity to any one of SEQ ID NO: 2-SEQ ID NO: 160. In some embodiments, the variant cytokine induces improved secondary structure in biological media. In some embodiments, the engineered cytokine comprises an amino acid substitution that results in reduced disorder of the engineered cytokine as compared to a wild type cytokine. Also disclosed herein are interleukins comprising modifications that result in reduced disorder, better folding and improved physical properties.

In some embodiments, the engineered IL-21 cytokine or a functional variant thereof includes the at least one amino acid substitution, at least two amino acid substitutions, at least three substitutions, at least four amino acid substitutions or at least five amino acid substitutions in a region including amino acid residues 30-135 of SEQ ID NO: 1 is at positions selected from: R34, H35, 137, R38, D44, 145, Q48, N70, A82, T89, G90, N92, E93, I95, I96, V98, K102, L103, K104, R105, P107, T110, N111, A112, G113, R114, R115, Q116, H118, R119, L120, or P133.

TABLE 1
IL Sequences

| SEQ | Poly | | |
|---|---|---|---|
| ID | peptide | | |
| NO | No. | Mutation | Sequence |
| 1 | 1 | wild type | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERIINVSIKKLKRKPPSTNAGRRQKHRLTCPSCDSYEKKPP |
| | | | KEFLERFKSLLQKMIHQHLSSRTHGSEDS |
| 2 | 2 | R34P | MRSSPGNMERIVICLMVIFLGTLVHKSSSQGQDPHMIRMRQLIDI |
| | | | VDQLKNYVNDLVPEFLPAPEDVETNCEWSAFSCFQKAQLKSAN |
| | | | TGNNERIINVSIKKLKRKPPSTNAGRRQKHRLTCPSCDSYEKKPP |
| | | | KEFLERFKSLLQKMIHQHLSSRTHGSEDS |
| 3 | 3 | R34W | MRSSPGNMERIVICLMVIFLGTLVHKSSSQGQDWHMIRMRQLIDI |
| | | | VDQLKNYVNDLVPEFLPAPEDVETNCEWSAFSCFQKAQLKSAN |
| | | | TGNNERIINVSIKKLKRKPPSTNAGRRQKHRLTCPSCDSYEKKPP |
| | | | KEFLERFKSLLQKMIHQHLSSRTHGSEDS |
| 4 | 4 | H35C | MRSSPGNMERIVICLMVIFLGTLVHKSSSQGQDRCMIRMRQLIDI |
| | | | VDQLKNYVNDLVPEFLPAPEDVETNCEWSAFSCFQKAQLKSAN |
| | | | TGNNERIINVSIKKLKRKPPSTNAGRRQKHRLTCPSCDSYEKKPP |
| | | | KEFLERFKSLLQKMIHQHLSSRTHGSEDS |
| 5 | 5 | H35F | MRSSPGNMERIVICLMVIFLGTLVHKSSSQGQDRFMIRMRQLIDI |
| | | | VDQLKNYVNDLVPEFLPAPEDVETNCEWSAFSCFQKAQLKSAN |
| | | | TGNNERIINVSIKKLKRKPPSTNAGRRQKHRLTCPSCDSYEKKPP |
| | | | KEFLERFKSLLQKMIHQHLSSRTHGSEDS |
| 6 | 6 | H35W | MRSSPGNMERIVICLMVIFLGTLVHKSSSQGQDRWMIRMRQLIDI |
| | | | VDQLKNYVNDLVPEFLPAPEDVETNCEWSAFSCFQKAQLKSAN |
| | | | TGNNERIINVSIKKLKRKPPSTNAGRRQKHRLTCPSCDSYEKKPP |
| | | | KEFLERFKSLLQKMIHQHLSSRTHGSEDS |
| 7 | 7 | H35Y | MRSSPGNMERIVICLMVIFLGTLVHKSSSQGQDRYMIRMRQLIDI |
| | | | VDQLKNYVNDLVPEFLPAPEDVETNCEWSAFSCFQKAQLKSAN |
| | | | TGNNERIINVSIKKLKRKPPSTNAGRRQKHRLTCPSCDSYEKKPP |
| | | | KEFLERFKSLLQKMIHQHLSSRTHGSEDS |
| 8 | 8 | I37K | MRSSPGNMERIVICLMVIFLGTLVHKSSSQGQDRHMKRMRQLID |
| | | | IVDQLKNYVNDLVPEFLPAPEDVETNCEWSAFSCFQKAQLKSAN |
| | | | TGNNERIINVSIKKLKRKPPSTNAGRRQKHRLTCPSCDSYEKKPP |
| | | | KEFLERFKSLLQKMIHQHLSSRTHGSEDS |
| 9 | 9 | I37R | MRSSPGNMERIVICLMVIFLGTLVHKSSSQGQDRHMRRMRQLID |
| | | | IVDQLKNYVNDLVPEFLPAPEDVETNCEWSAFSCFQKAQLKSAN |
| | | | TGNNERIINVSIKKLKRKPPSTNAGRRQKHRLTCPSCDSYEKKPP |
| | | | KEFLERFKSLLQKMIHQHLSSRTHGSEDS |
| 10 | 10 | I37H | MRSSPGNMERIVICLMVIFLGTLVHKSSSQGQDRHMHRMRQLID |
| | | | IVDQLKNYVNDLVPEFLPAPEDVETNCEWSAFSCFQKAQLKSAN |
| | | | TGNNERIINVSIKKLKRKPPSTNAGRRQKHRLTCPSCDSYEKKPP |
| | | | KEFLERFKSLLQKMIHQHLSSRTHGSEDS |
| 11 | 11 | R38F | MRSSPGNMERIVICLMVIFLGTLVHKSSSQGQDRHMIFMRQLIDI |
| | | | VDQLKNYVNDLVPEFLPAPEDVETNCEWSAFSCFQKAQLKSAN |
| | | | TGNNERIINVSIKKLKRKPPSTNAGRRQKHRLTCPSCDSYEKKPP |
| | | | KEFLERFKSLLQKMIHQHLSSRTHGSEDS |
| 12 | 12 | R38W | MRSSPGNMERIVICLMVIFLGTLVHKSSSQGQDRHMIWMRQLIDI |
| | | | VDQLKNYVNDLVPEFLPAPEDVETNCEWSAFSCFQKAQLKSAN |
| | | | TGNNERIINVSIKKLKRKPPSTNAGRRQKHRLTCPSCDSYEKKPP |
| | | | KEFLERFKSLLQKMIHQHLSSRTHGSEDS |
| 13 | 13 | D44K | MRSSPGNMERIVICLMVIFLGTLVHKSSSQGQDRHMIRMRQLIKI |
| | | | VDQLKNYVNDLVPEFLPAPEDVETNCEWSAFSCFQKAQLKSAN |
| | | | TGNNERIINVSIKKLKRKPPSTNAGRRQKHRLTCPSCDSYEKKPP |
| | | | KEFLERFKSLLQKMIHQHLSSRTHGSEDS |
| 14 | 14 | D44H | MRSSPGNMERIVICLMVIFLGTLVHKSSSQGQDRHMIRMRQLIHI |
| | | | VDQLKNYVNDLVPEFLPAPEDVETNCEWSAFSCFQKAQLKSAN |
| | | | TGNNERIINVSIKKLKRKPPSTNAGRRQKHRLTCPSCDSYEKKPP |
| | | | KEFLERFKSLLQKMIHQHLSSRTHGSEDS |
| 15 | 15 | I45V | MRSSPGNMERIVICLMVIFLGTLVHKSSSQGQDRHMIRMRQLID |
| | | | VVDQLKNYVNDLVPEFLPAPEDVETNCEWSAFSCFQKAQLKSA |
| | | | NTGNNERIINVSIKKLKRKPPSTNAGRRQKHRLTCPSCDSYEKKP |
| | | | PKEFLERFKSLLQKMIHQHLSSRTHGSEDS |
| 16 | 16 | I45T | MRSSPGNMERIVICLMVIFLGTLVHKSSSQGQDRHMIRMRQLIDT |
| | | | VDQLKNYVNDLVPEFLPAPEDVETNCEWSAFSCFQKAQLKSAN |
| | | | TGNNERIINVSIKKLKRKPPSTNAGRRQKHRLTCPSCDSYEKKPP |
| | | | KEFLERFKSLLQKMIHQHLSSRTHGSEDS |
| 17 | 17 | Q48T | MRSSPGNMERIVICLMVIFLGTLVHKSSSQGQDRHMIRMRQLIDI |
| | | | VDTLKNYVNDLVPEFLPAPEDVETNCEWSAFSCFQKAQLKSANT |
| | | | GNNERIINVSIKKLKRKPPSTNAGRRQKHRLTCPSCDSYEKKPPK |
| | | | EFLERFKSLLQKMIHQHLSSRTHGSEDS |
| 18 | 18 | Q48S | MRSSPGNMERIVICLMVIFLGTLVHKSSSQGQDRHMIRMRQLIDI |
| | | | VDSLKNYVNDLVPEFLPAPEDVETNCEWSAFSCFQKAQLKSANT |
| | | | GNNERIINVSIKKLKRKPPSTNAGRRQKHRLTCPSCDSYEKKPPK |
| | | | EFLERFKSLLQKMIHQHLSSRTHGSEDS |
| 19 | 19 | N70G | MRSSPGNMERIVICLMVIFLGTLVHKSSSQGQDRHMIRMRQLIDI |
| | | | VDQLKNYVNDLVPEFLPAPEDVETGCEWSAFSCFQKAQLKSAN |
| | | | TGNNERIINVSIKKLKRKPPSTNAGRRQKHRLTCPSCDSYEKKPP |
| | | | KEFLERFKSLLQKMIHQHLSSRTHGSEDS |
| 20 | 20 | A82I | MRSSPGNMERIVICLMVIFLGTLVHKSSSQGQDRHMIRMRQLIDI |
| | | | VDQLKNYVNDLVPEFLPAPEDVETNCEWSAFSCFQKIQLKSANT |
| | | | GNNERIINVSIKKLKRKPPSTNAGRRQKHRLTCPSCDSYEKKPPK |
| | | | EFLERFKSLLQKMIHQHLSSRTHGSEDS |
| 21 | 21 | A82R | MRSSPGNMERIVICLMVIFLGTLVHKSSSQGQDRHMIRMRQLIDI |
| | | | VDQLKNYVNDLVPEFLPAPEDVETNCEWSAFSCFQKRQLKSAN |
| | | | TGNNERIINVSIKKLKRKPPSTNAGRRQKHRLTCPSCDSYEKKPP |
| | | | KEFLERFKSLLQKMIHQHLSSRTHGSEDS |
| 22 | 22 | T89L | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | LGNNERIINVSIKKLKRKPPSTNAGRRQKHRLTCPSCDSYEKKPP |
| | | | KEFLERFKSLLQKMIHQHLSSRTHGSEDS |
| 23 | 23 | G90Y | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TYNNERIINVSIKKLKRKPPSTNAGRRQKHRLTCPSCDSYEKKPP |
| | | | KEFLERFKSLLQKMIHQHLSSRTHGSEDS |
| 24 | 24 | G90W | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TWNNERIINVSIKKLKRKPPSTNAGRRQKHRLTCPSCDSYEKKPP |
| | | | KEFLERFKSLLQKMIHQHLSSRTHGSEDS |
| 25 | 25 | N92L | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNLERIINVSIKKLKRKPPSTNAGRRQKHRLTCPSCDSYEKKPP |
| | | | KEFLERFKSLLQKMIHQHLSSRTHGSEDS |
| 26 | 26 | N92I | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNIERIINVSIKKLKRKPPSTNAGRRQKHRLTCPSCDSYEKKPPK |
| | | | EFLERFKSLLQKMIHQHLSSRTHGSEDS |
| 27 | 27 | N92M | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNMERIINVSIKKLKRKPPSTNAGRRQKHRLTCPSCDSYEKKPP |
| | | | KEFLERFKSLLQKMIHQHLSSRTHGSEDS |
| 28 | 28 | E93L | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNLRIINVSIKKLKRKPPSTNAGRRQKHRLTCPSCDSYEKKPP |
| | | | KEFLERFKSLLQKMIHQHLSSRTHGSEDS |
| 29 | 29 | I95K | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERKINVSIKKLKRKPPSTNAGRRQKHRLTCPSCDSYEKKPP |
| | | | KEFLERFKSLLQKMIHQHLSSRTHGSEDS |
| 30 | 30 | I95L | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERLINVSIKKLKRKPPSTNAGRRQKHRLTCPSCDSYEKKPP |
| | | | KEFLERFKSLLQKMIHQHLSSRTHGSEDS |
| 31 | 31 | I95R | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERRINVSIKKLKRKPPSTNAGRRQKHRLTCPSCDSYEKKPP |
| | | | KEFLERFKSLLQKMIHQHLSSRTHGSEDS |
| 32 | 32 | I96P | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERIPNVSIKKLKRKPPSTNAGRRQKHRLTCPSCDSYEKKPP |
| | | | KEFLERFKSLLQKMIHQHLSSRTHGSEDS |
| 33 | 33 | V98W | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERIINWSIKKLKRKPPSTNAGRRQKHRLTCPSCDSYEKKPP |
| | | | KEFLERFKSLLQKMIHQHLSSRTHGSEDS |
| 34 | 34 | V98Y | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERIINYSIKKLKRKPPSTNAGRRQKHRLTCPSCDSYEKKPP |
| | | | KEFLERFKSLLQKMIHQHLSSRTHGSEDS |
| 35 | 35 | K102T | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERIINVSIKTLKRKPPSTNAGRRQKHRLTCPSCDSYEKKPP |
| | | | KEFLERFKSLLQKMIHQHLSSRTHGSEDS |
| 36 | 36 | K102Y | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERIINVSIKYLKRKPPSTNAGRRQKHRLTCPSCDSYEKKPP |
| | | | KEFLERFKSLLQKMIHQHLSSRTHGSEDS |
| 37 | 37 | K102F | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERIINVSIKFLKRKPPSTNAGRRQKHRLTCPSCDSYEKKPP |
| | | | KEFLERFKSLLQKMIHQHLSSRTHGSEDS |
| 38 | 38 | L103P | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERIINVSIKKPKRKPPSTNAGRRQKHRLTCPSCDSYEKKPP |
| | | | KEFLERFKSLLQKMIHQHLSSRTHGSEDS |
| 39 | 39 | K104L | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERIINVSIKKLLRKPPSTNAGRRQKHRLTCPSCDSYEKKPP |
| | | | KEFLERFKSLLQKMIHQHLSSRTHGSEDS |
| 40 | 40 | R105W | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERIINVSIKKLKWKPPSTNAGRRQKHRLTCPSCDSYEKKPP |
| | | | KEFLERFKSLLQKMIHQHLSSRTHGSEDS |
| 41 | 41 | P107L | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERIINVSIKKLKRKLPSTNAGRRQKHRLTCPSCDSYEKKPP |
| | | | KEFLERFKSLLQKMIHQHLSSRTHGSEDS |
| 42 | 42 | P107I | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERIINVSIKKLKRKIPSTNAGRRQKHRLTCPSCDSYEKKPP |
| | | | KEFLERFKSLLQKMIHQHLSSRTHGSEDS |
| 43 | 43 | P107N | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERIINVSIKKLKRKNPSTNAGRRQKHRLTCPSCDSYEKKPP |
| | | | KEFLERFKSLLQKMIHQHLSSRTHGSEDS |
| 44 | 44 | P107W | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERIINVSIKKLKRKWPSTNAGRRQKHRLTCPSCDSYEKKPP |
| | | | KEFLERFKSLLQKMIHQHLSSRTHGSEDS |
| 45 | 45 | T110L | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERIINVSIKKLKRKPPSLNAGRRQKHRLTCPSCDSYEKKPP |
| | | | KEFLERFKSLLQKMIHQHLSSRTHGSEDS |
| 46 | 46 | N111L | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERIINVSIKKLKRKPPSTLAGRRQKHRLTCPSCDSYEKKPP |
| | | | KEFLERFKSLLQKMIHQHLSSRTHGSEDS |
| 47 | 47 | N111E | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERIINVSIKKLKRKPPSTEAGRRQKHRLTCPSCDSYEKKPP |
| | | | KEFLERFKSLLQKMIHQHLSSRTHGSEDS |
| 48 | 48 | N111W | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERIINVSIKKLKRKPPSTWAGRRQKHRLTCPSCDSYEKKPP |
| | | | KEFLERFKSLLQKMIHQHLSSRTHGSEDS |
| 49 | 49 | N111I | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERIINVSIKKLKRKPPSTIAGRRQKHRLTCPSCDSYEKKPPK |
| | | | EFLERFKSLLQKMIHQHLSSRTHGSEDS |
| 50 | 50 | N111M | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERIINVSIKKLKRKPPSTMAGRRQKHRLTCPSCDSYEKKPP |
| | | | KEFLERFKSLLQKMIHQHLSSRTHGSEDS |
| 51 | 51 | A112I | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERIINVSIKKLKRKPPSTNIGRRQKHRLTCPSCDSYEKKPPK |
| | | | EFLERFKSLLQKMIHQHLSSRTHGSEDS |
| 52 | 52 | A112M | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERIINVSIKKLKRKPPSTNMGRRQKHRLTCPSCDSYEKKPP |
| | | | KEFLERFKSLLQKMIHQHLSSRTHGSEDS |
| 53 | 53 | A112W | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERIINVSIKKLKRKPPSTNWGRRQKHRLTCPSCDSYEKKPP |
| | | | KEFLERFKSLLQKMIHQHLSSRTHGSEDS |
| 54 | 54 | G113Y | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERIINVSIKKLKRKPPSTNAYRRQKHRLTCPSCDSYEKKPP |
| | | | KEFLERFKSLLQKMIHQHLSSRTHGSEDS |
| 55 | 55 | G113L | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERIINVSIKKLKRKPPSTNALRRQKHRLTCPSCDSYEKKPP |
| | | | KEFLERFKSLLQKMIHQHLSSRTHGSEDS |
| 56 | 56 | G113W | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERIINVSIKKLKRKPPSTNAWRRQKHRLTCPSCDSYEKKPP |
| | | | KEFLERFKSLLQKMIHQHLSSRTHGSEDS |
| 57 | 57 | G113A | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERIINVSIKKLKRKPPSTNAARRQKHRLTCPSCDSYEKKPP |
| | | | KEFLERFKSLLQKMIHQHLSSRTHGSEDS |
| 58 | 58 | G113Q | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERIINVSIKKLKRKPPSTNAQRRQKHRLTCPSCDSYEKKPP |
| | | | KEFLERFKSLLQKMIHQHLSSRTHGSEDS |
| 59 | 59 | R114L | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERIINVSIKKLKRKPPSTNAGLRQKHRLTCPSCDSYEKKPP |
| | | | KEFLERFKSLLQKMIHQHLSSRTHGSEDS |
| 60 | 60 | R115F | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERIINVSIKKLKRKPPSTNAGRFQKHRLTCPSCDSYEKKPP |
| | | | KEFLERFKSLLQKMIHQHLSSRTHGSEDS |
| 61 | 61 | R115Y | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERIINVSIKKLKRKPPSTNAGRYQKHRLTCPSCDSYEKKPP |
| | | | KEFLERFKSLLQKMIHQHLSSRTHGSEDS |
| 62 | 62 | Q116L | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERIINVSIKKLKRKPPSTNAGRRLKHRLTCPSCDSYEKKPP |
| | | | KEFLERFKSLLQKMIHQHLSSRTHGSEDS |
| 63 | 63 | H118P | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERIINVSIKKLKRKPPSTNAGRRQKPRLTCPSCDSYEKKPP |
| | | | KEFLERFKSLLQKMIHQHLSSRTHGSEDS |
| 64 | 64 | R119M | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERIINVSIKKLKRKPPSTNAGRRQKHMLTCPSCDSYEKKPP |
| | | | KEFLERFKSLLQKMIHQHLSSRTHGSEDS |
| 65 | 65 | R119P | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERIINVSIKKLKRKPPSTNAGRRQKHPLTCPSCDSYEKKPP |
| | | | KEFLERFKSLLQKMIHQHLSSRTHGSEDS |
| 66 | 66 | L120Y | MRSSPGNMERIVICLMVIFLGTLVHKSSSQGQDRHMIRMRQLIDI |
| | | | VDQLKNYVNDLVPEFLPAPEDVETNCEWSAFSCFQKAQLKSAN |
| | | | TGNNERIINVSIKKLKRKPPSTNAGRRQKHRYTCPSCDSYEKKPP |
| | | | KEFLERFKSLLQKMIHQHLSSRTHGSEDS |
| 67 | 67 | R38F | MRSSPGNMERIVICLMVIFLGTLVHKSSSQGQDRHMIFMRQLIDI |
| | | P107L | VDQLKNYVNDLVPEFLPAPEDVETNCEWSAFSCFQKAQLKSAN |
| | | | TGNNERIINVSIKKLKRKLPSTNAGRRQKHRLTCPSCDSYEKKPP |
| | | | KEFLERFKSLLQKMIHQHLSSRTHGSEDS |
| 68 | 68 | R38W | MRSSPGNMERIVICLMVIFLGTLVHIWMRQLIDIVDQLKNYVND |
| | | P107L | LVPEFLPAPEDVETNCEWSAFSCFQKAQLKSANTGNNERIINVSI |
| | | | KKLKRKLPSTNAGRRQKHRLTCPSCDSYEKKPPKEFLERFKSLL |
| | | | QKMIHQHLSSRTHGSEDS |
| 69 | 69 | R38E | MRSSPGNMERIVICLMVIFLGTLVHKSSSQGQDRHMIEMRQLIDI |
| | | P107L | VDQLKNYVNDLVPEFLPAPEDVETNCEWSAFSCFQKAQLKSAN |
| | | | TGNNERIINVSIKKLKRKLPSTNAGRRQKHRLTCPSCDSYEKKPP |
| | | | KEFLERFKSLLQKMIHQHLSSRTHGSEDS |
| 70 | 70 | R105W | MRSSPGNMERIVICLMVIFLGTLVHIRMRQLIDIVDQLKNYVNDL |
| | | P107L | VPEFLPAPEDVETNCEWSAFSCFQKAQLKSANTGNNERIINVSIK |
| | | | KLKWKLPSTNAGRRQKHRLTCPSCDSYEKKPPKEFLERFKSLLQ |
| | | | KMIHQHLSSRTHGSEDS |
| 71 | 71 | R38F, | MRSSPGNMERIVICLMVIFLGTLVHIFMRQLIDIVDQLKNYVNDL |
| | | R105W, | VPEFLPAPEDVETNCEWSAFSCFQKAQLKSANTGNNERIINVSIK |
| | | P107L | KLKWKLPSTNAGRRQKHRLTCPSCDSYEKKPPKEFLERFKSLLQ |
| | | | KMIHQHLSSRTHGSEDS |
| 72 | 72 | R38W, | MRSSPGNMERIVICLMVIFLGTLVHKSSSQGQDRHMIWMRQLIDI |
| | | R105W, | VDQLKNYVNDLVPEFLPAPEDVETNCEWSAFSCFQKAQLKSAN |
| | | P107L | TGNNERIINVSIKKLKWKLPSTNAGRRQKHRLTCPSCDSYEKKPP |
| | | | KEFLERFKSLLQKMIHQHLSSRTHGSEDS |
| 73 | 73 | A82R, | MRSSPGNMERIVICLMVIFLGTLVHIRMRQLIDIVDQLKNYVNDL |
| | | 196P, | VPEFLPAPED VETNCEWSAFSCFQKRQLKSANTGNNERIPNVSIK |
| | | L103P | KPKRKPPSTNAGRRQKHRLTCPSCDSYEKKPPKEFLERFKSLLQK |
| | | | MIHQHLSSRTHGSEDS |
| 74 | 74 | T89L, | MRSSPGNMERIVICLMVIFLGTLVHKSSSQGQDRHMIRMRQLIDI |
| | | N92M, | VDQLKNYVNDLVPEFLPAPEDVETNCEWSAFSCFQKAQLKSAN |
| | | P133 L | LGNMERIINVSIKKLKRKPPSTNAGRRQKHRLTCPSCDSYEKKPL |
| | | | KEFLERFKSLLQKMIHQHLSSRTHGSEDS |
| 75 | 75 | N92L, | MRSSPGNMERIVICLMVIFLGTLVHKSSSQGQDRHMIRMRQLIDI |
| | | P107I, | VDQLKNYVNDLVPEFLPAPEDVETNCEWSAFSCFQKAQLKSAN |
| | | T110L | TGNLERIINVSIKKLKRKIPSLNAGRRQKHRLTCPSCDSYEKKPPK |
| | | | EFLERFKSLLQKMIHQHLSSRTHGSEDS |
| 76 | 76 | N92I, | MRSSPGNMERIVICLMVIFLGTLVHKSSSQGQDRHMIRMRQLIDI |
| | | G113L, | VDQLKNYVNDLVPEFLPAPEDVETNCEWSAFSCFQKAQLKSAN |
| | | Q116L | TGNIERIINVSIKKLKRKPPSTNALRRLKHRLTCPSCDSYEKKPPK |
| | | | EFLERFKSLLQKMIHQHLSSRTHGSEDS |
| 77 | 77 | E93L, | MRSSPGNMERIVICLMVIFLGTLVHKSSSQGQDRHMIRMRQLIDI |
| | | K104L, | VDQLKNYVNDLVPEFLPAPEDVETNCEWSAFSCFQKAQLKSAN |
| | | P107L | TGNNLRIINVSIKKLLRKLPSTNAGRRQKHRLTCPSCDSYEKKPP |
| | | | KEFLERFKSLLQKMIHQHLSSRTHGSEDS |
| 78 | 78 | P107W, | MRSSPGNMERIVICLMVIFLGTLVHKSSSQGQDRHMIRMRQLIDI |
| | | G113Y, | VDQLKNYVNDLVPEFLPAPEDVETNCEWSAFSCFQKAQLKSAN |
| | | R114L | TGNNERIINVSIKKLKRKWPSTNAYLRQKHRLTCPSCDSYEKKPP |
| | | | KEFLERFKSLLQKMIHQHLSSRTHGSEDS |
| 79 | 79 | N92I, | MRSSPGNMERIVICLMVIFLGTLVHKSSSQGQDRHMIRMRQLIDI |
| | | E93L, | VDQLKNYVNDLVPEFLPAPEDVETNCEWSAFSCFQKAQLKSAN |
| | | P107N, | TGNILRIINVSIKKLKRKNPSTEAGRRLKHRLTCPSCDSYEKKPPK |
| | | N111E, | EFLERFKSLLQKMIHQHLSSRTHGSEDS |
| | | Q116L | |
| 80 | 80 | H35C, | MRSSPGNMERIVICLMVIFLGTLVHKSSSQGQDRCMIRMRQLIDI |
| | | E93L, | VDQLKNYVNDLVPEFLPAPEDVETNCEWSAFSCFQKAQLKSAN |
| | | K104L, | TGNNLRIINVSIKKLLRKLPSTLAGRRQKHRLTCPSCDSYEKKPP |
| | | P107L, | KEFLERFKSLLQKMIHQHLSSRTHGSEDS |
| | | N111L | |

n.a. 81 indicates not applicable.

TABLE 2
Further modified IL Sequences

| | | Mutation | |
|---|---|---|---|
| | | (with ref | |
| SEQ | Poly | to SEQ ID | |
| ID | peptide | NO: 1 | |
| NO | No. | positions) | Sequence |
| 81 | 81 | Wild type | QGQDRHMIRMRQLIDIVDQLKNYVNDLVPEFLPAPEDVETNCE |
| | | | WSAFSCFQKAQLKSANTGNNERIINVSIKKLKRKPPSTNAGRRQ |
| | | | KHRLTCPSCDSYEKKPPKE |
| 82 | 82 | R34P | QGQDPHMIRMRQLIDIVDQLKNYVNDLVPEFLPAPEDVETNCEW |
| | | | SAFSCFQKAQLKSANTGNNERIINVSIKKLKRKPPSTNAGRRQKH |
| | | | RLTCPSCDSYEKKPPKE |
| 83 | 83 | R34W | QGQDWHMIRMRQLIDIVDQLKNYVNDLVPEFLPAPEDVETNCE |
| | | | WSAFSCFQKAQLKSANTGNNERIINVSIKKLKRKPPSTNAGRRQ |
| | | | KHRLTCPSCDSYEKKPPKE |
| 83 | 84 | H35C | QGQDRCMIRMRQLIDIVDQLKNYVNDLVPEFLPAPEDVETNCEW |
| | | | SAFSCFQKAQLKSANTGNNERIINVSIKKLKRKPPSTNAGRRQKH |
| | | | RLTCPSCDSYEKKPPKE |
| 85 | 85 | H35F | QGQDRFMIRMRQLIDIVDQLKNYVNDLVPEFLPAPEDVETNCEW |
| | | | SAFSCFQKAQLKSANTGNNERIINVSIKKLKRKPPSTNAGRRQKH |
| | | | RLTCPSCDSYEKKPPKE |
| 86 | 86 | H35W | QGQDRWMIRMRQLIDIVDQLKNYVNDLVPEFLPAPEDVETNCE |
| | | | WSAFSCFQKAQLKSANTGNNERIINVSIKKLKRKPPSTNAGRRQ |
| | | | KHRLTCPSCDSYEKKPPKE |
| 87 | 87 | H35Y | QGQDRYMIRMRQLIDIVDQLKNYVNDLVPEFLPAPEDVETNCEW |
| | | | SAFSCFQKAQLKSANTGNNERIINVSIKKLKRKPPSTNAGRRQKH |
| | | | RLTCPSCDSYEKKPPKE |
| 88 | 88 | I37K | QGQDRHMKRMRQLIDIVDQLKNYVNDLVPEFLPAPEDVETNCE |
| | | | WSAFSCFQKAQLKSANTGNNERIINVSIKKLKRKPPSTNAGRRQ |
| | | | KHRLTCPSCDSYEKKPPKE |
| 89 | 89 | I37R | QGQDRHMHRMRQLIDIVDQLKNYVNDLVPEFLPAPEDVETNCE |
| | | | WSAFSCFQKAQLKSANTGNNERIINVSIKKLKRKPPSTNAGRRQ |
| | | | KHRLTCPSCDSYEKKPPKE |
| 90 | 90 | I37H | QGQDRHMHRMRQLIDIVDQLKNYVNDLVPEFLPAPEDVETNCE |
| | | | WSAFSCFQKAQLKSANTGNNERIINVSIKKLKRKPPSTNAGRRQ |
| | | | KHRLTCPSCDSYEKKPPKE |
| 91 | 91 | R38F | QGQDRHMIFMRQLIDIVDQLKNYVNDLVPEFLPAPEDVETNCEW |
| | | | SAFSCFQKAQLKSANTGNNERIINVSIKKLKRKPPSTNAGRRQKH |
| | | | RLTCPSCDSYEKKPPKE |
| 92 | 92 | R38W | QGQDRHMIWMRQLIDIVDQLKNYVNDLVPEFLPAPEDVETNCE |
| | | | WSAFSCFQKAQLKSANTGNNERIINVSIKKLKRKPPSTNAGRRQ |
| | | | KHRLTCPSCDSYEKKPPKE |
| 93 | 93 | D44K | QGQDRHMIRMRQLIKIVDQLKNYVNDLVPEFLPAPEDVETNCE |
| | | | WSAFSCFQKAQLKSANTGNNERIINVSIKKLKRKPPSTNAGRRQ |
| | | | KHRLTCPSCDSYEKKPPKE |
| 94 | 94 | D44H | QGQDRHMIRMRQLIHIVDQLKNYVNDLVPEFLPAPEDVETNCE |
| | | | WSAFSCFQKAQLKSANTGNNERIINVSIKKLKRKPPSTNAGRRQ |
| | | | KHRLTCPSCDSYEKKPPKE |
| 95 | 95 | I45V | QGQDRHMIRMRQLIDVVDQLKNYVNDLVPEFLPAPEDVETNCE |
| | | | WSAFSCFQKAQLKSANTGNNERIINVSIKKLKRKPPSTNAGRRQ |
| | | | KHRLTCPSCDSYEKKPPKE |
| 96 | 96 | I45T | QGQDRHMIRMRQLIDTVDQLKNYVNDLVPEFLPAPEDVETNCE |
| | | | WSAFSCFQKAQLKSANTGNNERIINVSIKKLKRKPPSTNAGRRQ |
| | | | KHRLTCPSCDSYEKKPPKE |
| 97 | 97 | Q48T | QGQDRHMIRMRQLIDIVDTLKNYVNDLVPEFLPAPEDVETNCEW |
| | | | SAFSCFQKAQLKSANTGNNERIINVSIKKLKRKPPSTNAGRRQKH |
| | | | RLTCPSCDSYEKKPPKE |
| 98 | 98 | Q48S | QGQDRHMIRMRQLIDIVDSLKNYVNDLVPEFLPAPEDVETNCEW |
| | | | SAFSCFQKAQLKSANTGNNERIINVSIKKLKRKPPSTNAGRRQKH |
| | | | RLTCPSCDSYEKKPPKE |
| 99 | 99 | N70G | QGQDRHMIRMRQLIDIVDQLKNYVNDLVPEFLPAPEDVETGCE |
| | | | WSAFSCFQKAQLKSANTGNNERIINVSIKKLKRKPPSTNAGRRQ |
| | | | KHRLTCPSCDSYEKKPPKE |
| 100 | 100 | A82I | QGQDRHMIRMRQLIDIVDQLKNYVNDLVPEFLPAPEDVETNCE |
| | | | WSAFSCFQKIQLKSANTGNNERIINVSIKKLKRKPPSTNAGRRQK |
| | | | HRLTCPSCDSYEKKPPKE |
| 101 | 101 | A82R | QGQDRHMIRMRQLIDIVDQLKNYVNDLVPEFLPAPEDVETNCE |
| | | | WSAFSCFQKRQLKSANTGNNERIINVSIKKLKRKPPSTNAGRRQ |
| | | | KHRLTCPSCDSYEKKPPKE |
| 102 | 102 | T89L | QGQDRHMIRMRQLIDIVDQLKNYVNDLVPEFLPAPEDVETNCE |
| | | | WSAFSCFQKAQLKSANLGNNERIINVSIKKLKRKPPSTNAGRRQ |
| | | | KHRLTCPSCDSYEKKPPKE |
| 103 | 103 | G90Y | QGQDRHMIRMRQLIDIVDQLKNYVNDLVPEFLPAPEDVETNCE |
| | | | WSAFSCFQKAQLKSANTYNNERIINVSIKKLKRKPPSTNAGRRQ |
| | | | KHRLTCPSCDSYEKKPPKE |
| 104 | 104 | G90W | QGQDRHMIRMRQLIDIVDQLKNYVNDLVPEFLPAPEDVETNCE |
| | | | WSAFSCFQKAQLKSANTWNNERIINVSIKKLKRKPPSTNAGRRQ |
| | | | KHRLTCPSCDSYEKKPPKE |
| 405 | 105 | N92L | QGQDRHMIRMRQLIDIVDQLKNYVNDLVPEFLPAPEDVETNCE |
| | | | WSAFSCFQKAQLKSANTGNLERIINVSIKKLKRKPPSTNAGRRQ |
| | | | KHRLTCPSCDSYEKKPPKE |
| 106 | 106 | N92I | QGQDRHMIRMRQLIDIVDQLKNYVNDLVPEFLPAPEDVETNCE |
| | | | WSAFSCFQKAQLKSANTGNIERIINVSIKKLKRKPPSTNAGRRQK |
| | | | HRLTCPSCDSYEKKPPKE |
| 107 | 107 | N92M | QGQDRHMIRMRQLIDIVDQLKNYVNDLVPEFLPAPEDVETNCE |
| | | | WSAFSCFQKAQLKSANTGNMERIINVSIKKLKRKPPSTNAGRRQ |
| | | | KHRLTCPSCDSYEKKPPKE |
| 108 | 108 | E93L | QGQDRHMIRMRQLIDIVDQLKNYVNDLVPEFLPAPEDVETNCE |
| | | | WSAFSCFQKAQLKSANTGNNLRIINVSIKKLKRKPPSTNAGRRQ |
| | | | KHRLTCPSCDSYEKKPPKE |
| 109 | 109 | I95K | QGQDRHMIRMRQLIDIVDQLKNYVNDLVPEFLPAPEDVETNCE |
| | | | WSAFSCFQKAQLKSANTGNNERKINVSIKKLKRKPPSTNAGRRQ |
| | | | KHRLTCPSCDSYEKKPPKE |
| 110 | 110 | I95L | QGQDRHMIRMRQLIDIVDQLKNYVNDLVPEFLPAPEDVETNCE |
| | | | WSAFSCFQKAQLKSANTGNNERLINVSIKKLKRKPPSTNAGRRQ |
| | | | KHRLTCPSCDSYEKKPPKE |
| 111 | 111 | I95R | QGQDRHMIRMRQLIDIVDQLKNYVNDLVPEFLPAPEDVETNCE |
| | | | WSAFSCFQKAQLKSANTGNNERRINVSIKKLKRKPPSTNAGRRQ |
| | | | KHRLTCPSCDSYEKKPPKE |
| 112 | 1112 | I96P | QGQDRHMIRMRQLIDIVDQLKNYVNDLVPEFLPAPEDVETNCE |
| | | | WSAFSCFQKAQLKSANTGNNERIPNVSIKKLKRKPPSTNAGRRQ |
| | | | KHRLTCPSCDSYEKKPPKE |
| 113 | 113 | V98W | QGQDRHMIRMRQLIDIVDQLKNYVNDLVPEFLPAPEDVETNCE |
| | | | WSAFSCFQKAQLKSANTGNNERIINWSIKKLKRKPPSTNAGRRQ |
| | | | KHRLTCPSCDSYEKKPPKE |
| 114 | 114 | V98Y | QGQDRHMIRMRQLIDIVDQLKNYVNDLVPEFLPAPEDVETNCE |
| | | | WSAFSCFQKAQLKSANTGNNERIINYSIKKLKRKPPSTNAGRRQ |
| | | | KHRLTCPSCDSYEKKPPKE |
| 115 | 115 | K102T | QGQDRHMIRMRQLIDIVDQLKNYVNDLVPEFLPAPEDVETNCE |
| | | | WSAFSCFQKAQLKSANTGNNERIINVSIKTLKRKPPSTNAGRRQ |
| | | | KHRLTCPSCDSYEKKPPKE |
| 116 | 116 | K102Y | QGQDRHMIRMRQLIDIVDQLKNYVNDLVPEFLPAPEDVETNCE |
| | | | WSAFSCFQKAQLKSANTGNNERIINVSIKYLKRKPPSTNAGRRQ |
| | | | KHRLTCPSCDSYEKKPPKE |
| 117 | 117 | K102F | QGQDRHMIRMRQLIDIVDQLKNYVNDLVPEFLPAPEDVETNCE |
| | | | WSAFSCFQKAQLKSANTGNNERIINVSIKFLKRKPPSTNAGRRQ |
| | | | KHRLTCPSCDSYEKKPPKE |
| 118 | 118 | L103P | QGQDRHMIRMRQLIDIVDQLKNYVNDLVPEFLPAPEDVETNCE |
| | | | WSAFSCFQKAQLKSANTGNNERIINVSIKKPKRKPPSTNAGRRQ |
| | | | KHRLTCPSCDSYEKKPPKE |
| 119 | 119 | K104L | QGQDRHMIRMRQLIDIVDQLKNYVNDLVPEFLPAPEDVETNCE |
| | | | WSAFSCFQKAQLKSANTGNNERIINVSIKKLLRKPPSTNAGRRQ |
| | | | KHRLTCPSCDSYEKKPPKE |
| 120 | 120 | R105W | QGQDRHMIRMRQLIDIVDQLKNYVNDLVPEFLPAPEDVETNCE |
| | | | WSAFSCFQKAQLKSANTGNNERIINVSIKKLKWKPPSTNAGRRQ |
| | | | KHRLTCPSCDSYEKKPPKE |
| 121 | 121 | P107L | QGQDRHMIRMRQLIDIVDQLKNYVNDLVPEFLPAPEDVETNCE |
| | | | WSAFSCFQKAQLKSANTGNNERIINVSIKKLKRKLPSTNAGRRQ |
| | | | KHRLTCPSCDSYEKKPPKE |
| 122 | 122 | P107I | QGQDRHMIRMRQLIDIVDQLKNYVNDLVPEFLPAPEDVETNCE |
| | | | WSAFSCFQKAQLKSANTGNNERIINVSIKKLKRKIPSTNAGRRQK |
| | | | HRLTCPSCDSYEKKPPKE |
| 123 | 123 | P107N | QGQDRHMIRMRQLIDIVDQLKNYVNDLVPEFLPAPEDVETNCE |
| | | | WSAFSCFQKAQLKSANTGNNERIINVSIKKLKRKNPSTNAGRRQ |
| | | | KHRLTCPSCDSYEKKPPKE |
| 124 | 124 | P107W | QGQDRHMIRMRQLIDIVDQLKNYVNDLVPEFLPAPEDVETNCE |
| | | | WSAFSCFQKAQLKSANTGNNERIINVSIKKLKRKWPSTNAGRRQ |
| | | | KHRLTCPSCDSYEKKPPKE |
| 125 | 125 | T110L | QGQDRHMIRMRQLIDIVDQLKNYVNDLVPEFLPAPEDVETNCE |
| | | | WSAFSCFQKAQLKSANTGNNERIINVSIKKLKRKPPSLNAGRRQ |
| | | | KHRLTCPSCDSYEKKPPKE |
| 126 | 126 | N111L | QGQDRHMIRMRQLIDIVDQLKNYVNDLVPEFLPAPEDVETNCE |
| | | | WSAFSCFQKAQLKSANTGNNERIINVSIKKLKRKPPSTLAGRRQ |
| | | | KHRLTCPSCDSYEKKPPKE |
| 127 | 127 | N111E | QGQDRHMIRMRQLIDIVDQLKNYVNDLVPEFLPAPEDVETNCE |
| | | | WSAFSCFQKAQLKSANTGNNERIINVSIKKLKRKPPSTEAGRRQ |
| | | | KHRLTCPSCDSYEKKPPKE |
| 128 | 128 | N111W | QGQDRHMIRMRQLIDIVDQLKNYVNDLVPEFLPAPEDVETNCE |
| | | | WSAFSCFQKAQLKSANTGNNERIINVSIKKLKRKPPSTWAGRRQ |
| | | | KHRLTCPSCDSYEKKPPKE |
| 129 | 129 | N111I | QGQDRHMIRMRQLIDIVDQLKNYVNDLVPEFLPAPEDVETNCE |
| | | | WSAFSCFQKAQLKSANTGNNERIINVSIKKLKRKPPSTIAGRRQK |
| | | | HRLTCPSCDSYEKKPPKE |
| 130 | 130 | N111M | QGQDRHMIRMRQLIDIVDQLKNYVNDLVPEFLPAPEDVETNCE |
| | | | WSAFSCFQKAQLKSANTGNNERIINVSIKKLKRKPPSTMAGRRQ |
| | | | KHRLTCPSCDSYEKKPPKE |
| 131 | 131 | A112I | QGQDRHMIRMRQLIDIVDQLKNYVNDLVPEFLPAPEDVETNCE |
| | | | WSAFSCFQKAQLKSANTGNNERIINVSIKKLKRKPPSTNIGRRQK |
| | | | HRLTCPSCDSYEKKPPKE |
| 132 | 132 | A112M | QGQDRHMIRMRQLIDIVDQLKNYVNDLVPEFLPAPEDVETNCE |
| | | | WSAFSCFQKAQLKSANTGNNERIINVSIKKLKRKPPSTNMGRRQ |
| | | | KHRLTCPSCDSYEKKPPKE |
| 133 | 133 | A112W | QGQDRHMIRMRQLIDIVDQLKNYVNDLVPEFLPAPEDVETNCE |
| | | | WSAFSCFQKAQLKSANTGNNERIINVSIKKLKRKPPSTNWGRRQ |
| | | | KHRLTCPSCDSYEKKPPKE |
| 134 | 134 | G113Y | QGQDRHMIRMRQLIDIVDQLKNYVNDLVPEFLPAPEDVETNCE |
| | | | WSAFSCFQKAQLKSANTGNNERIINVSIKKLKRKPPSTNAYRRQ |
| | | | KHRLTCPSCDSYEKKPPKE |
| 135 | 135 | G113L | QGQDRHMIRMRQLIDIVDQLKNYVNDLVPEFLPAPEDVETNCE |
| | | | WSAFSCFQKAQLKSANTGNNERIINVSIKKLKRKPPSTNALRRQ |
| | | | KHRLTCPSCDSYEKKPPKE |
| 136 | 136 | G113W | QGQDRHMIRMRQLIDIVDQLKNYVNDLVPEFLPAPEDVETNCE |
| | | | WSAFSCFQKAQLKSANTGNNERIINVSIKKLKRKPPSTNAWRRQ |
| | | | KHRLTCPSCDSYEKKPPKE |
| 137 | 137 | G113A | QGQDRHMIRMRQLIDIVDQLKNYVNDLVPEFLPAPEDVETNCE |
| | | | WSAFSCFQKAQLKSANTGNNERIINVSIKKLKRKPPSTNAARRQ |
| | | | KHRLTCPSCDSYEKKPPKE |
| 138 | 138 | G113Q | QGQDRHMIRMRQLIDIVDQLKNYVNDLVPEFLPAPEDVETNCE |
| | | | WSAFSCFQKAQLKSANTGNNERIINVSIKKLKRKPPSTNAQRRQ |
| | | | KHRLTCPSCDSYEKKPPKE |
| 139 | 139 | R114L | QGQDRHMIRMRQLIDIVDQLKNYVNDLVPEFLPAPEDVETNCE |
| | | | WSAFSCFQKAQLKSANTGNNERIINVSIKKLKRKPPSTNAGLRQ |
| | | | KHRLTCPSCDSYEKKPPKE |
| 140 | 140 | R115F | QGQDRHMIRMRQLIDIVDQLKNYVNDLVPEFLPAPEDVETNCE |
| | | | WSAFSCFQKAQLKSANTGNNERIINVSIKKLKRKPPSTNAGRFQ |
| | | | KHRLTCPSCDSYEKKPPKE |
| 141 | 141 | R115Y | QGQDRHMIRMRQLIDIVDQLKNYVNDLVPEFLPAPEDVETNCE |
| | | | WSAFSCFQKAQLKSANTGNNERIINVSIKKLKRKPPSTNAGRYQ |
| | | | KHRLTCPSCDSYEKKPPKE |
| 142 | 142 | Q116L | QGQDRHMIRMRQLIDIVDQLKNYVNDLVPEFLPAPEDVETNCE |
| | | | WSAFSCFQKAQLKSANTGNNERIINVSIKKLKRKPPSTNAGRRL |
| | | | KHRLTCPSCDSYEKKPPKE |
| 143 | 143 | H118P | QGQDRHMIRMRQLIDIVDQLKNYVNDLVPEFLPAPEDVETNCE |
| | | | WSAFSCFQKAQLKSANTGNNERIINVSIKKLKRKPPSTNAGRRQ |
| | | | KPRLTCPSCDSYEKKPPKE |
| 144 | 144 | R119M | QGQDRHMIRMRQLIDIVDQLKNYVNDLVPEFLPAPEDVETNCE |
| | | | WSAFSCFQKAQLKSANTGNNERIINVSIKKLKRKPPSTNAGRRQ |
| | | | KHMLTCPSCDSYEKKPPKE |
| 145 | 145 | R119P | QGQDRHMIRMRQLIDIVDQLKNYVNDLVPEFLPAPEDVETNCE |
| | | | WSAFSCFQKAQLKSANTGNNERIINVSIKKLKRKPPSTNAGRRQ |
| | | | KHPLTCPSCDSYEKKPPKE |
| 146 | 146 | L120Y | QGQDRHMIRMRQLIDIVDQLKNYVNDLVPEFLPAPEDVETNCE |
| | | | WSAFSCFQKAQLKSANTGNNERIINVSIKKLKRKPPSTNAGRRQ |
| | | | KHRYTCPSCDSYEKKPPKE |
| 147 | 147 | R38F | QGQDRHMIFMRQLIDIVDQLKNYVNDLVPEFLPAPEDVETNCEW |
| | | P107L | SAFSCFQKAQLKSANTGNNERIINVSIKKLKRKLPSTNAGRRQK |
| | | | HRLTCPSCDSYEKKPPKE |
| 148 | 148 | R38W | IWMRQLIDIVDQLKNYVNDLVPEFLPAPEDVETNCEWSAFSCFQ |
| | | P107L | KAQLKSANTGNNERIINVSIKKLKRKLPSTNAGRRQKHRLTCPSC |
| | | | DSYEKKPPKE |
| 149 | 149 | R38E | QGQDRHMIEMRQLIDIVDQLKNYVNDLVPEFLPAPEDVETNCEW |
| | | P107L | SAFSCFQKAQLKSANTGNNERIINVSIKKLKRKLPSTNAGRRQK |
| | | | HRLTCPSCDSYEKKPPKE |
| 150 | 150 | R105W | IRMRQLIDIVDQLKNYVNDLVPEFLPAPEDVETNCEWSAFSCFQK |
| | | P107L | AQLKSANTGNNERIINVSIKKLKWKLPSTNAGRRQKHRLTCPSC |
| | | | DSYEKKPPKE |
| 151 | 151 | R38F, | IFMRQLIDIVDQLKNYVNDLVPEFLPAPEDVETNCEWSAFSCFQK |
| | | R105W, | AQLKSANTGNNERIINVSIKKLKWKLPSTNAGRRQKHRLTCPSC |
| | | P107L | DSYEKKPPKE |
| 152 | 152 | R38W, | QGQDRHMIWMRQLIDIVDQLKNYVNDLVPEFLPAPEDVETNCE |
| | | R105W, | WSAFSCFQKAQLKSANTGNNERIINVSIKKLKWKLPSTNAGRRQ |
| | | P107L | KHRLTCPSCDSYEKKPPKE |
| 153 | 153 | A82R, | IRMRQLIDIVDQLKNYVNDLVPEFLPAPEDVETNCEWSAFSCFQK |
| | | 196P, and | RQLKSANTGNNERIPNVSIKKPKRKPPSTNAGRRQKHRLTCPSCD |
| | | L103P | SYEKKPPKE |
| 154 | 154 | T89L, | QGQDRHMIRMRQLIDIVDQLKNYVNDLVPEFLPAPEDVETNCE |
| | | N92M, | WSAFSCFQKAQLKSANLGNMERIINVSIKKLKRKPPSTNAGRRQ |
| | | P133 L | KHRLTCPSCDSYEKKPLKE |
| 155 | 155 | N92L, | QGQDRHMIRMRQLIDIVDQLKNYVNDLVPEFLPAPEDVETNCE |
| | | P107I, | WSAFSCFQKAQLKSANTGNLERIINVSIKKLKRKIPSLNAGRRQK |
| | | T110L | HRLTCPSCDSYEKKPPKE |
| 156 | 156 | N92I, | QGQDRHMIRMRQLIDIVDQLKNYVNDLVPEFLPAPEDVETNCE |
| | | G113L, | WSAFSCFQKAQLKSANTGNIERIINVSIKKLKRKPPSTNALRRLK |
| | | Q116L | HRLTCPSCDSYEKKPPKE |
| 157 | 157 | E93L, | QGQDRHMIRMRQLIDIVDQLKNYVNDLVPEFLPAPEDVETNCE |
| | | K104L, | WSAFSCFQKAQLKSANTGNNLRIINVSIKKLLRKLPSTNAGRRQ |
| | | P107L | KHRLTCPSCDSYEKKPPKE |
| 158 | 158 | P107W, | QGQDRHMIRMRQLIDIVDQLKNYVNDLVPEFLPAPEDVETNCE |
| | | G113Y, | WSAFSCFQKAQLKSANTGNNERIINVSIKKLKRKWPSTNAYLRQ |
| | | R114L | KHRLTCPSCDSYEKKPPKE |
| 159 | 159 | N92I, | QGQDRHMIRMRQLIDIVDQLKNYVNDLVPEFLPAPEDVETNCE |
| | | E93L, | WSAFSCFQKAQLKSANTGNILRIINVSIKKLKRKNPSTEAGRRLK |
| | | P107N, | HRLTCPSCDSYEKKPPKE |
| | | N111E, | |
| | | Q116L | |
| 160 | 160 | H35C, | QGQDRCMIRMRQLIDIVDQLKNYVNDLVPEFLPAPEDVETNCEW |
| | | E93L, | SAFSCFQKAQLKSANTGNNLRIINVSIKKLLRKLPSTLAGRRQKH |
| | | K104L, | RLTCPSCDSYEKKPPKE |
| | | P107L, | |
| | | N111L | |

In some embodiments are interleukins comprising a tag amino acid sequence comprising at least 2 amino acid residue repeats. In some embodiments, the tag is a histidine tag. In some embodiments, the tag comprises: HHHHHHHHHH (SEQ ID NO: 173). In some embodiments, the tag is a 10 Histidine tag. In some embodiments, the interleukin is an interleukin described in TABLE 3.

TABLE 3
IL-21 variants, sample numbers and respective mutations. Sequences
contain the C-terminal 10 × His tag for affinity purification.

| | Polypeptide | | |
|---|---|---|---|
| SEQ ID NO. | No. | Mutation | Sequence |
| 161 | 161 | n.a. | QGQDRHMIRMRQLIDIVDQLKNYVNDLV |
| | | | PEFLPAPEDVETNCEWSAFSCFQKAQLKS |
| | | | ANTGNNERIINVSIKKLKRKPPSTNAGRR |
| | | | QKHRLTCPSCDSYEKKPPKEFLERFKSLLQ |
| | | | KMIHQHLSSRTHGSEDSHHHHHHHHHH |
| 162 | 162 | N92M | QGQDRHMIRMRQLIDIVDQLKNYVNDLV |
| | | | PEFLPAPEDVETNCEWSAFSCFQKAQLKS |
| | | | ANTGNMERIINVSIKKLKRKPPSTNAGRR |
| | | | QKHRLTCPSCDSYEKKPPKEFLERFKSLLQ |
| | | | KMIHQHLSSRTHGSEDSHHHHHHHHHH |
| 163 | 163 | P107L | QGQDRHMIRMRQLIDIVDQLKNYVNDLV |
| | | | PEFLPAPEDVETNCEWSAFSCFQKAQLKS |
| | | | ANTGNNERIINVSIKKLKRKLPSTNAGRR |
| | | | QKHRLTCPSCDSYEKKPPKEFLERFKSLLQ |
| | | | KMIHQHLSSRTHGSEDSHHHHHHHHHH |
| 164 | 164 | A82I | QGQDRHMIRMRQLIDIVDQLKNYVNDLV |
| | | | PEFLPAPEDVETNCEWSAFSCFQKIQLKSA |
| | | | NTGNNERIINVSIKKLKRKPPSTNAGRRQ |
| | | | KHRLTCPSCDSYEKKPPKEFLERFKSLLQK |
| | | | MIHQHLSSRTHGSEDSHHHHHHHHHH |
| 165 | 165 | E93L, | QGQDRHMIRMRQLIDIVDQLKNYVNDLV |
| | | K104L, and | PEFLPAPEDVETNCEWSAFSCFQKAQLKS |
| | | P107L | ANTGNNLRIINVSIKKLLRKLPSTNAGRRQ |
| | | | KHRLTCPSCDSYEKKPPKEFLERFKSLLQK |
| | | | MIHQHLSSRTHGSEDSHHHHHHHHHH |
| 166 | 166 | N92L, | QGQDRHMIRMRQLIDIVDQLKNYVNDLV |
| | | P1071, and | PEFLPAPEDVETNCEWSAFSCFQKAQLKS |
| | | T110L | ANTGNLERIINVSIKKLKRKIPSLNAGRRQ |
| | | | KHRLTCPSCDSYEKKPPKEFLERFKSLLQK |
| | | | MIHQHLSSRTHGSEDSHHHHHHHHHH |
| 167 | 167 | N92I, | QGQDRHMIRMRQLIDIVDQLKNYVNDLV |
| | | G113L, and | PEFLPAPEDVETNCEWSAFSCFQKAQLKS |
| | | Q116L | ANTGNIERIINVSIKKLKRKPPSTNALRRL |
| | | | KHRLTCPSCDSYEKKPPKEFLERFKSLLQK |
| | | | MIHQHLSSRTHGSEDSHHHHHHHHHH |
| 168 | 168 | T89L, | QGQDRHMIRMRQLIDIVDQLKNYVNDLV |
| | | N92M, and | PEFLPAPEDVETNCEWSAFSCFQKAQLKS |
| | | P133L | ANLGNMERIINVSIKKLKRKPPSTNAGRR |
| | | | QKHRLTCPSCDSYEKKPLKEFLERFKSLL |
| | | | QKMIHQHLSSRTHGSEDSHHHHHHHHHH |
| 169 | 169 | N921, E93L, | QGQDRHMIRMRQLIDIVDQLKNYVNDLV |
| | | P107N, | PEFLPAPEDVETNCEWSAFSCFQKAQLKS |
| | | N111E, and | ANTGNILRIINVSIKKLKRKNPSTEAGRRL |
| | | Q116L | KHRLTCPSCDSYEKKPPKEFLERFKSLLQK |
| | | | MIHQHLSSRTHGSEDSHHHHHHHHHH |
| 170 | 170 | H35C, | QGQDRCMIRMRQLIDIVDQLKNYVNDLV |
| | | E93L, | PEFLPAPEDVETNCEWSAFSCFQKAQLKS |
| | | K104L, | ANTGNNLRIINVSIKKLLRKLPSTLAGRRQ |
| | | P107L, and | KHRLTCPSCDSYEKKPPKEFLERFKSLLQK |
| | | N111L | MIHQHLSSRTHGSEDSHHHHHHHHHH |
| 171 | 171 | A82R, 196P, | QGQDRHMIRMRQLIDIVDQLKNYVNDLV |
| | | and L103P | PEFLPAPEDVETNCEWSAFSCFQKRQLKS |
| | | | ANTGNNERIPNVSIKKPKRKPPSTNAGRR |
| | | | QKHRLTCPSCDSYEKKPPKEFLERFKSLLQ |
| | | | KMIHQHLSSRTHGSEDSHHHHHHHHHH |
| 172 | 172 | P107W, | QGQDRHMIRMRQLIDIVDQLKNYVNDLV |
| | | G113Y, | PEFLPAPEDVETNCEWSAFSCFQKAQLKS |
| | | R114L | ANTGNNERIINVSIKKLKRKWPSTNAYLR |
| | | | QKHRLTCPSCDSYEKKPPKEFLERFKSLLQ |
| | | | KMIHQHLSSRTHGSEDSHHHHHHHHHH |

Any composition or pharmaceutical composition herein comprise, consist of, or consist essentially of any engineered or engineered cytokine sequence comprising any SEQ ID NO herein. Embodiments herein can comprise an engineered cytokine sequence having a sequence having at least about 70, 75, 80, 85, 90, 95, 96, 97, 98, or 99 percent identity to the sequence of any SEQ ID NO herein and/or at least about 80, 85, 90, 95, 96, 97, 98, or 99 percent length to the sequence of any SEQ ID NO herein. In some embodiments, an engineered interleukin is used interchangeably with an engineered interleukin.

In some embodiments, a composition is provided comprising amino acids based on SEQ ID NO: 1 comprising 1, 2, 3, 4, or more amino acid substitutions. In some embodiments, a composition is provided comprising amino acids based on SEQ ID NO: 2-SEQ ID NO: 160 comprising 0, 1, 2, 3, 4, or more amino acid substitutions. In some embodiments, an engineered cytokine comprising a sequence of any one of SEQ ID NO: 2-SEQ ID NO: 160 demonstrates an increased stability in the protein's secondary structure.

In some embodiments, the engineered interleukin comprises an amino acid substitution. Also disclosed herein are engineered interleukins comprising a modification in a disordered region that reduces the level of disorder and enhances the secondary or tertiary structure of the interleukin. In some embodiments, the engineered interleukin comprises an amino acid substitution in a region of disorder of the engineered interleukin. In some embodiments, the engineered interleukin comprises an amino acid substitution in a region complimentary to a region of disorder of the engineered interleukin.

In some embodiments, the amino acid substitution provides for increased stability of the engineered interleukin compared to a wild type interleukin. A region of disorder of an engineered interleukin described herein includes regions with no defined secondary or tertiary protein structure. In some embodiments, an engineered interleukin described herein, in comparison with a wild type interleukin, lacks hydrogen bonding exhibited by the wild type interleukin.

In some embodiments, the composition of the present disclosure provides for a composition comprising an engineered IL-21 cytokine or functional fragment thereof, wherein the increased stability is characterized by an increased thermal stability of the engineered IL-21 cytokine compared to that of the wild type IL-21 cytokine. In some embodiments, the increased thermal stability is determined by comparing the melting temperature of the engineered IL-21 cytokine or the wild type IL-21 cytokine. In some embodiments, the melting temperature of the engineered IL-21 cytokine is increased by about 2 degrees Celsius compared to the wild type cytokine. In some embodiments, the melting temperature of the engineered IL-21 cytokine is increased by about 3 degrees Celsius. In some embodiments, the melting temperature of the engineered IL-21 cytokine is increased by about 4 degrees Celsius. In some embodiments, the melting temperature of the engineered IL-21 cytokine is increased by about 5 degrees Celsius. In some embodiments, the melting temperature of the engineered IL-21 cytokine is increased by about 6 degrees Celsius. In some embodiments, the melting temperature of the engineered IL-21 cytokine is increased by about 7 degrees Celsius. In some embodiments, the melting temperature of the engineered IL-21 cytokine is increased by about 8 degrees Celsius. In some embodiments, the melting temperature of the engineered IL-21 cytokine is increased by about 9 degrees Celsius. In some embodiments, the melting temperature of the engineered IL-21 cytokine is increased by about 10 degrees Celsius. In some embodiments, the melting temperature of the engineered IL-21 cytokine is increased by about 11 degrees Celsius. In some embodiments, the melting temperature of the engineered IL-21 cytokine is increased by about 12 degrees Celsius. In some embodiments, the melting temperature of the engineered IL-21 cytokine is increased by about 13 degrees Celsius. In some embodiments, the melting temperature of the engineered IL-21 cytokine is increased by about 14 degrees Celsius. In some embodiments, the melting temperature of the engineered IL-21 cytokine is increased by or about 15 degrees Celsius. In some embodiments, the melting temperature of the engineered IL-21 cytokine is increased by about 7 degrees Celsius compared to that of the wild type IL-21 cytokine. In some embodiments, the melting temperature of the engineered IL-21 cytokine is increased by about 11 degrees Celsius compared to that of the wild type IL-21 cytokine.

In some embodiments, the composition of the present disclosure provides for a composition, wherein the increased stability is further characterized by an increased resistance to pepsin digestion of the engineered IL-21 cytokine compared to that of the wild type IL-21 cytokine, wherein the pepsin digestion produces peptide fragments, and wherein the increased resistance to pepsin digestion is evaluated by quantifying the peptide fragments measured by mass spectrometry. In some embodiments, the increased resistance is about 10%. In some embodiments, the increased resistance is about 12%. In some embodiments, the increased resistance is about 14%. In some embodiments, the increased resistance is about 16%. In some embodiments, the increased resistance is about 18%. In some embodiments, the increased resistance is about 20%. In some embodiments, the increased resistance is about 22%. In some embodiments, the increased resistance is about 12%. In some embodiments, the increased resistance is about 20%.

In some embodiments, the composition of the present disclosure provides for a composition, wherein the increased stability is further characterized by an increased structural rigidity of the engineered IL-21 cytokine compared to the wild type IL-21 cytokine. In some embodiments, the increased structural rigidity is measured by an increased proton-deuterium exchange rate of the engineered IL-21 cytokine compared to that of the wild type IL-21 cytokine when measured by hydrogen deuterium exchange mass spectrometry (HDX-MS).

In some embodiments, the composition of the present disclosure provides for a composition, wherein the increased stability is further characterized by an increased compactness of the engineered IL-21 cytokine compared to that of the wild type IL-21 cytokine as measured by dynamic light scattering (DLS). In some embodiments, the DLS determines an average particle diameter of the engineered IL-21 cytokine or the wild type IL-21 cytokine, wherein the average particle diameter of the engineered IL-21 cytokine is decreased by about 0.10 nm. In some embodiments, the average particle diameter of the engineered IL-21 cytokine is decreased by about 0.15 nm. In some embodiments, the average particle diameter of the engineered IL-21 cytokine is decreased by about 0.20 nm. In some embodiments, the average particle diameter of the engineered IL-21 cytokine is decreased by about 0.25. In some embodiments, the average particle diameter of the engineered IL-21 cytokine is decreased by about 0.20 nm compared to that of the wild type IL-21 cytokine. In some embodiments, the average particle diameter of the engineered IL-21 cytokine is decreased by about 0.25 nm compared to that of the wild type IL-21 cytokine.

In some embodiments, the composition of the present disclosure provides for a composition, wherein the increased stability is further characterized by an increased yield in an expression system of the engineered IL-21 cytokine compared to that of the wild type IL-21 cytokine. In some embodiments, the increased yield is about 2-fold. In some embodiments, the increased yield is about 3-fold. In some embodiments, the increased yield is about 4-fold. In some embodiments, the increased yield is about 5-fold. In some embodiments, the increased yield is about 6-fold. In some embodiments, the increased yield is about 7-fold. In some embodiments, the increased yield is about 8-fold. In some embodiments, the increased yield is about 9-fold. In some embodiments, the increased yield is about-10 fold. In some embodiments, the increased yield is about 11-fold. In some embodiments, the increased yield is about 12-fold. In some embodiments, the increased yield is about 13-fold. In some embodiments, the increased yield is about 14-fold. In some embodiments, the increased yield is about 15-fold. In some embodiments, the increased yield is about 4-fold. In some embodiments, the increased yield is about 11-fold. In some embodiments, the increased yield is about 13-fold.

In some embodiments, the composition of the present disclosure provides for a composition, wherein the engineered IL-21 cytokine induces a higher STAT3 phosphorylation in a cell line compared to that of the wild type IL-21 cytokine.

In some embodiments, the composition of the present disclosure provides for a composition, wherein the engineered cytokine has a lower affinity to an IL-21 receptor compared to that of the wild type IL-21 cytokine. In some embodiments, the lower affinity to the IL-21 receptor is lower by about 80-fold In some embodiments, the lower affinity to the IL-21 receptor is lower by about 90-fold In some embodiments, the lower affinity to the IL-21 receptor is lower by about 100-fold In some embodiments, the lower affinity to the IL-21 receptor is lower by about 110-fold In some embodiments, the lower affinity to the IL-21 receptor is lower by about 120-fold In some embodiments, the lower affinity to the IL-21 receptor is lower by about 130-fold. In some embodiments, the lower affinity to the IL-21 receptor is lower by about 100-fold. In some embodiments, the lower affinity to the IL-21 receptor is lower by about 110-fold.

In some embodiments, the composition of the present disclosure provides for a composition, wherein the engineered IL-21 cytokine results in an improved exposure following administration to a subject relative to the wild type IL-21 cytokine, as measured by a greater area under curve (AUC) for the engineered IL-21 cytokine.

Engineered interleukins described herein include mutant, or variant interleukin. Variant interleukin sequences described herein can comprise engineered interleukins comprising sequences with 90%, 91%, 92%, 93%, 94%, 95%, 96%, 97%, 98%, 99%, 99.5%, 99.9% and 100% sequence identity to the sequences disclosed in SEQ ID NO: 2-SEQ ID NO: 160. In some embodiments, an engineered interleukin herein comprises a sequence having at least 80%, 85%, 90%, 95% or more sequence identity to any one of SEQ ID NO: 2-SEQ ID NO: 160. In some embodiments, the variant interleukin induces secondary structure in biological media. In some embodiments, the engineered interleukin comprises an amino acid substitution that results in decreased disorder of the engineered interleukin as compared to a wild type interleukin. Also disclosed herein are interleukins comprising modifications that result in decreased disorder. In some embodiments are two interleukins which form a complex with a defined tertiary structure (e.g., IL-21 and an IL-21 variant). In some embodiments, the engineered interleukin comprises an amino acid deletion. In some embodiments, the engineered interleukin comprises an amino acid insertion. In some embodiments, the amino acid deletion is of an amino acid in a disordered region of the interleukin. In some embodiments, the amino acid insertion is of an amino acid in a disordered region of the interleukin. In some embodiments, the amino acid deletion is of an amino acid in a region complimentary to a disordered region of the interleukin. In some embodiments, the amino acid insertion is of an amino acid in a region complimentary to a disordered region of the interleukin. Also described herein are engineered interleukins with one or more mutations to the amino acid sequence of the engineered interleukins resulting in variant interleukin sequences. Exemplary mutations of engineered interleukins described herein are shown in TABLE 4. In some embodiments, substitutions of engineered interleukins described herein are with an amino acid also having a charged, polar or non-polar side chain as the residue being placed. An engineered interleukin described herein can have a substitution at one amino acid on the polypeptide. An engineered interleukin described herein can have a substitution at more than one amino acid on the polypeptide. TABLE 4 provides exemplary substitutions of a wild type amino acid residue for a variant residue. Amino acid residue number refers to the location of the amino acid from N-terminus to C-terminus of the polypeptide.

TABLE 4
Exemplary Interleukin Mutations

| Amino acid | Wild type | |
|---|---|---|
| residue No. | residue | Variant residue(s) |
| 34 | R | P, W, |
| 35 | H | C, F, W, G, Y, P |
| 37 | I | K, R, H, |
| 38 | R | F, W, E |
| 44 | D | K, H |
| 45 | I | V, T |
| 48 | Q | T, S |
| 70 | N | G |
| 82 | A | I, M, A, P, Y, W, M, R |
| 89 | T | L, I, A, P, Y, W, M |
| 90 | G | Y, W |
| 92 | N | M, L, I, V, A, P, Y, W |
| 93 | E | K, H, R, L |
| 95 | I | K, L, R |
| 96 | I | P, L, I, A, Y, W, M |
| 98 | V | W, Y |
| 102 | K | T, Y, F, |
| 103 | L | P, L, I, A, Y |
| 104 | K | L, A, P, Y, W, M |
| 105 | R | W |
| 107 | P | L, I, A, P, Y, W, M, N |
| 110 | T | L, I, A, P, Y, W, M |
| 111 | N | E, K, H, R, L, W, I, M |
| 112 | A | I, M, W, |
| 113 | G | L, I, A, P, Y, W, M, Q |
| 114 | R | L, I, A, P, Y, W, M |
| 115 | R | F, Y |
| 116 | Q | L, I, A, P, Y, W, M |
| 118 | H | P |
| 119 | R | M, P |
| 120 | L | Y |
| 133 | P | L, I, A, P, Y, W, M |

Amino acid codes provided here are as follows (name/three letter code/single letter code): alanine/ala/A; arginine/arg/R; asparagine/asn/N; aspartic acid/asp/D; asparagine or aspartic acid/asx/B; cysteine/cys/C; glutamic acid/glu/E; glutamine/gln/Q; glutamine or glutamic acid/glx/Z; glycine/gly/G; histidine/his/H; isoleucine/ile/I; leucine/leu/L; lysine/lys/K: methionine/met/M: phenylalanine/phe/F: proline/pro/P: serine/ser/S; threonine/thr/T: tryptophan/trp/W: tyrosine/tyr/Y: valine/val/V.

In some embodiments, the sequence of the cytokine comprises, consists of, or consists essentially of any one of the sequences in TABLE 1 or TABLE 2.

In some embodiments, a composition or pharmaceutical composition comprises an engineered cytokine that comprises, consists of, or consists essentially of one of the sequences in TABLE 1 or TABLE 2 and an excipient, diluent, carrier, or any combination of these, any or all of which may be pharmaceutically acceptable. In some embodiments, the pharmaceutical composition, further includes a solubilizing agent and an excipient. In some embodiments, the excipient includes one or more of a buffering agent, a stabilizer, an antioxidant, a binder, a diluent, a dispersing agent, a rate controlling agent, a lubricant, a glidant, a disintegrant, a plasticizer, a preservative, or any combinations thereof. In some embodiments, the pharmaceutical composition is formulated for parenteral or enteral administration. In some embodiments, pharmaceutical composition is in a lyophilized form.

Any of the sequences described in TABLE 1 or TABLE 2 may be encoded by a corresponding nucleic acid sequence. In some embodiments, provided herein is a composition comprising a nucleic acid encoding for an engineered cytokine having a protein sequence described in TABLE 1 or TABLE 2. In some embodiments, provided herein is a composition comprising a nucleic acid encoding for an engineered cytokine having a protein sequence with any one of SEQ ID NO: 2-SEQ ID NO: 160. In some embodiments, provided herein is a composition comprising a nucleic acid encoding for an engineered cytokine having a protein sequence with at least 90%, 91%, 92%, 93%, 94%, 95%, 96%, 97%, 98%, 99%, 99.5%, 99.9% and 100% any one of SEQ ID NO: 2-SEQ ID NO: 160. In some embodiments, the present disclosure also provides for a polynucleotide encoding the engineered IL-21 cytokine or a functional fragment thereof described herein or the engineered cytokine described herein. In some embodiments, there is provided a polynucleotide comprising a nucleotide sequence encoding the engineered IL-21 cytokine or a functional fragment thereof described herein or the engineered cytokine described herein. In some embodiments, there is provided a vector including the polynucleotide encoding the engineered IL-21 cytokine or a functional fragment thereof described herein or the engineered cytokine described herein. In some embodiments, the vector is a lentiviral vector. In some embodiments, there is provided a host cell including the vector disclosed herein. In some embodiments, the host cell is an immune cell. In some embodiments, the immune cell is a T cell or an NK cell. In some embodiments, the host cell further expresses a chimeric antigen receptor T cell. In some embodiments, there is provided an oncolytic virus including an exogenous nucleic acid that codes for the engineered IL-21 cytokine or a functional fragment thereof described herein or the engineered cytokine described herein. In some embodiments, the oncolytic virus includes a lentivirus.

In some embodiments, a composition or pharmaceutical composition can comprise an engineered interleukin that comprises at least a portion of a sequence of TABLE 1 or TABLE 2. In some embodiments, an engineered interleukin here can have about: 80, 85, 90, 95, 96, 97, 98, or 99% identity to any SEQ ID NO herein, for example, SEQ ID NO: 2-SEQ ID NO: 160. In some embodiments, a composition or pharmaceutical composition comprises an engineered interleukin having from about 100 to about 120 amino acid residues, about 110 to about 130 amino acid residues, about 120 to about 140 amino acid residues, or about 130 to about 150 amino acid residues. In some embodiments, a composition or pharmaceutical composition herein can comprise a cytokine comprising an engineered interleukin wherein the engineered interleukin can have from about 100 to about 120 amino acid residues, about 110 to about 130 amino acid residues, about 120 to about 140 amino acid residues, or about 130 to about 150 amino acid residues. In some embodiments herein, an engineered interleukin can have about: 80, 85, 90, 95, 96, 97, 98, or 99% sequence length to any SEQ ID NO herein, for example, SEQ ID NO: 2-SEQ ID NO: 160. In some embodiments, engineered interleukin does not arise from an epigenetic modification. In some embodiments, each engineered interleukin is unmethylated. In some embodiments, each engineered interleukin is pegylated.

In some embodiments, an engineered interleukin is present, for example in a composition or a pharmaceutical composition, in an amount from: about 1 ng to about 100 ng, about 100 ng to about 500 ng, about 500 ng to about 1 mg, about 1 mg to about 500 mg, about 500 mg to about 1000 mg, about 1000 mg to about 5000 mg, about 5000 mg to about 10000 mg, about 10000 to 25000 mg, or about 25000 to 50000 mg.

In some embodiments, a composition and/or pharmaceutical composition can regulate effector T-cell activity. In some embodiments, a composition and/or pharmaceutical composition can regulate regulatory T-cell activity. In some embodiments, a composition and/or pharmaceutical composition can regulate monocyte activity.

### Conjugate and Fusion Compositions

Further provided herein are engineered cytokines conjugated to a functional moiety. In some embodiments, conjugation of a cytokine can occur by providing a nucleic acid that encodes for a cytokine, an amino acid linker, and a second chemical or enzyme moiety or, conjugation of a cytokine can occur via chemical conjugation. Conjugating engineered cytokines result in enhanced biological properties and other activity profile measures including: i) targeted cytotoxicity, ii) half-life, iii) biological activity, iv) specificity, v) stability; and/or vi) targeted delivery. In some embodiments, the engineered cytokine is conjugated to at least one of: i) a toxin, ii) a fusion protein, iii) an antibody, or iv) another chemical, protein, or polymer. Fusion proteins include, for example, Fc fusion proteins and albumin fusion proteins. Cytokine-albumin fusion proteins can exhibit increased biological activity and half-life properties. The term, “fusion protein,” as used herein, includes a protein comprising at least two heterologous polypeptides. The fusion protein may comprise one or more effector proteins and effector partners. In some instances, an effector protein and effector partner are not found connected to one another as a native protein or complex that occurs together in nature. In some embodiments are cytokine-Fc fusion proteins that exhibit targeted cytotoxicity properties. Another important aspect of functional engineered cytokines are their properties resulting in enhanced targeted delivery. Targeted delivery properties can also be tailored using immunocytokines. Immunocytokines are molecules that combine a tumor directed antibody, a cytotoxic drug, and an engineered cytokine described herein. In some cases, the engineered cytokine is a pro-inflammatory cytokine. In some embodiments are multivalent cytokine fusions with enhanced specificity and potency through avidity: In some cases, the multivalent cytokine fusion comprises multiple binding domains resulting in enhanced avidity: In some cases, immunocytokine fusion proteins (e.g., IL-21-αHSA or IL-21-αFcRn) are described which display enhanced for half-life extension properties. In some cases, immunocytokine fusion proteins are described which display enhanced targeted delivery properties. Also described herein are cytokine-cell conjugates. Cytokine-cell conjugates include T-cell fusion moieties which allows for local, concentrated activity of otherwise toxic anti-tumor cytokines. Also described herein are cytokine-drug conjugates. Cytokine-drug conjugates are similar to cytokine-toxin conjugates which link an engineered cytokine described herein to a drug or drug-like molecule. The drug portion of a cytokine-drug conjugate include compounds and biologic therapeutics such as antibodies, anti-cancer drugs, anti-tumor drugs, anti-inflammatory drugs, or pro-inflammatory drugs.

### Delivery Systems

In some embodiments, compositions or pharmaceutical compositions described herein are administered or contacted for treatment of a skin or a tissue, for example of a subject or a subject in need thereof. In some embodiments, delivery is intravenous.

In some embodiments, the compositions and/or pharmaceutical compositions described herein are administered once, twice, three, four, five, six, seven, eight, nine, ten, or multiple times. In some embodiments, compositions and/or pharmaceutical compositions described herein are administered directly to or contacted directly or indirectly to immune cells.

In some embodiments, compositions and/or pharmaceutical compositions described herein are formulated in extended-release formulations, wherein the engineered interleukin is to a tissue over a defined duration of time. In some embodiments the duration of time is at least about: 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 15 hours, at least about 20 hours, at least about 24 hours, at least about 2 2 days, at least about 3 days, at least about 4 days, at least about 5 days, at least about 6 days, at least about 7 days wherein a day is 24 hours, at least about 2 weeks, at least about 3 weeks, at least about 4 weeks, at least about 8 weeks, at least about 12 weeks, at least about 16 weeks, at least about 20 weeks, at least about 24 weeks, or for as long as necessary or desired.

In some embodiments, the compositions and/or pharmaceutical compositions herein further comprises a booster. In some embodiments the booster is a protein. In some embodiments the protein is a boosting protein. In some embodiments the boosting protein is a second interleukin protein. In some embodiments, the interleukin protein is a lymphocyte.

### Pharmacological Carriers and Diluents

In some embodiments, pharmacologically acceptable carriers and pharmaceutically acceptable carriers may be referred to interchangeably herein. Exemplary pharmaceutically acceptable carriers include but are not limited to buffered solutions. In some embodiments, a buffered solution can be a solution that resists changes in pH when acid or alkali is added to it. In some embodiments, a buffered solution is or comprises phosphate buffered saline (PBS). In some embodiments, a carrier or a pharmaceutically acceptable carrier can be or include a penetrant. In some cases, a carrier can be a substrate used in the process of drug delivery. In some cases, a carrier can contribute to a composition's or pharmaceutically acceptable composition's attributes such as stability, biopharmaceutical profile, appearance, and/or patient acceptability. In some cases, a carrier or pharmaceutically acceptable carrier can be or comprise an organic excipient. Excipients include functional and/or non-functional ingredients in a composition or a pharmaceutical composition. In some cases, an excipient or a pharmaceutically acceptable excipient can comprise an oil, water, an aqueous solution, an acid, a salt, an alcohol, a carbohydrate, a sugar (i.e., a cyclodextrin), a buffer, a powder, a filler, a gum, a wax (e.g., carnauba, cetyl esters, microcrystalline, nonionic emulsifying, white, yellow), or any combination thereof.

In some cases, a carrier or a diluent or pharmaceutically acceptable carrier or diluent can be or comprise a solid such as a filling agent used in the production of a pill, for example lactose or another carbohydrate.

In some embodiments, the release and/or administration of a composition or pharmaceutical composition described herein is facilitated by a delivery system. In some embodiments, the delivery system requires at least one administration or contacting. In some embodiments, the delivery system can be administered or contacted with a subject or a subject in need thereof more than once, for example 2, 3, 4, 5, 6, 7, 8, 9, or 10 times or more. In some embodiments, the delivery system requires multiple administrations.

In some embodiments, the delivery system is or can comprise a polymer-based system. In some embodiments, the polymer-based system is selected from at least one of: a poly(lactideglycolide), a copolyoxalate, a polycaprolactone, a polyesteramide, a polyorthoester, a polyhydroxy butyric acid, a polyanhydride, and any combination thereof.

### Pharmaceutical Compositions

The compositions and pharmaceutical compositions herein, and/or the engineered interleukins, and/or any therapeutic or further therapeutic herein can be formulated as neutral or salt forms, including as a pharmaceutically acceptable salt. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc. In some embodiments, a salt or a pharmaceutically acceptable salt can comprise an HCl salt, an ascorbic acid salt, a mandelic acid salt, an aspartic acid salt, a carbonic acid salt, a citric acid salt, a formic acid salt, a glutamic acid salt, a lactic acid salt, a lauric acid salt, a maleic acid salt, a palmitic acid salt, a phosphoric acid salt, or any combination thereof. In some embodiments, a salt or a pharmaceutically acceptable salt can include, but is not limited to, a metal salt such as sodium salt, potassium salt, cesium salt and the like: an alkaline earth metal salt such as calcium salt, magnesium salt and the like: an organic amine salt such as a triethylamine salt, a pyridine salt, a picoline salt, an ethanolamine salt, a triethanolamine salt, a dicyclohexylamine salt, an N,N′-dibenzylethylenediamine salt and the like: an inorganic acid salt such as hydrochloride, hydrobromide, phosphate, sulphate, and the like: an organic acid salt such as citrate, lactate, tartrate, maleate, fumarate, mandelate, acetate, dichloroacetate, trifluoroacetate, oxalate, formate, and the like; a sulfonate such as methanesulfonate, benzenesulfonate, p-toluenesulfonate, and the like; and/or an amino acid salt such as arginate, asparginate, glutamate, and the like.

The compositions and pharmaceutical compositions disclosed herein can comprise a preservative, e.g., a compound which can be added to essentially reduce bacterial and/or fungal action or presence in or on any composition or pharmaceutical composition herein. Examples of preservatives include but are not limited to octadecyldimethylbenzyl ammonium chloride, hexamethonium chloride, benzalkoniurn chloride (a mixture of alkylbenzyldimethylammonium chlorides in which the alkyl groups are long-chain compounds), and benzethonium chloride.

A composition or pharmaceutical composition herein can be formulated to be compatible with its intended route of administration. Examples of routes of administration include, but are not limited to, topical, systemic, parenteral, e.g., intravenous, intradermal, subcutaneous, oral, intranasal (e.g., inhalation), intratumoral, transdermal (e.g., topical), transmucosal, and rectal administration. In some embodiments, a composition or pharmaceutical composition can be formulated as a composition or pharmaceutical composition adapted for intravenous, subcutaneous, intramuscular, oral, intranasal, transdermal, or topical administration to a human being or subject or subject in need thereof. Compositions and pharmaceutical compositions for intravenous administration can be solutions in sterile isotonic aqueous buffer. Compositions and pharmaceutical compositions here can be sterile or aseptic.

Compositions and pharmaceutical compositions herein may also include a solubilizing agent and/or a local anesthetic such as lignocaine or a pharmaceutically acceptable salt of any of these, for example, to case pain at the site of the administration, contacting, or injection. In some embodiments, the methods of the disclosure can comprise administration of a composition formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions, or emulsions in oily or aqueous vehicles, and may contain agents such as suspending, stabilizing, and/or dispersing agents. Alternatively, the active ingredient may be in powder form for constitution with a suitable vehicle (e.g., sterile pyrogen-free water) before use.

In some embodiments, a composition or a pharmaceutical composition comprises a surfactant. Surfactants can lower the surface tension of a liquid, the interfacial tension between two liquids, or that between a liquid and a solid. The surfactant can be a detergent, a wetting agent, an emulsifier, a foaming agent, a dispersant, or any combination thereof. In some embodiments, the surfactant can be a polysorbate-type emulsifier. In some embodiments, the polysorbate is a PEG (polyethylene glycol)-ylated sorbitan esterified with one or more fatty acids. In some embodiments, the surfactant is selected from: Polysorbate 20 (polyoxyethylene 20 sorbitan monolaurate), Polysorbate 60, Polysorbate 80, or any combination thereof.

In other embodiments, compositions and pharmaceutical compositions provided herein can be provided in an oral form, a transdermal form, an oil formulation, an edible food, or a food substrate, an aqueous dispersion, an emulsion, an oil-in-water emulsion, a water-in-oil emulsion, a solution, a suspension, an elixir, a gel, a syrup, an aerosol, a mist, a powder, a pill, a tablet, a lozenge, a gel, a lotion, a paste, a formulated stick, a balm, a cream, an ointment, or comprised in a bandage or a dressing.

In some embodiments, compositions and/or pharmaceutical compositions described herein comprise an engineered interleukin. In some embodiments, the engineered interleukin comprises an amino acid sequence from TABLE 1 or TABLE 2, or any one of SEQ ID NO: 2-SEQ ID NO: 160.

Provided herein are also kits comprising an engineered interleukin, and/or compositions or pharmaceutical compositions containing an engineered interleukin disclosed herein. The kits can include packaging, instructions, and/or a container. The kits can comprise a further therapeutic, which can be comprised in composition or a pharmaceutical composition, or comprised in the kit separately from the composition or the pharmaceutical composition. In some embodiments, the kits can contain additional compositions used to generate various formulation precursors.

### Therapeutic Applications

In some embodiments, compositions herein are used for the treatment of a disease or condition in a subject or a subject in need thereof. In some embodiments the disease or condition is a health-related, a health condition associated with damaged cells, a population of tumorous cells, or a cancer.

In some embodiments, the present disclosure provides a method of inducing cell death, the method comprising contacting a cell with the composition or pharmaceutical composition disclosed herein. In some embodiments, the cell is selected from: a lymphocyte cell, a B lymphocyte cell, or an MC116 cell. In some embodiments, the present disclosure provides a method of reducing cancer cell growth, the method comprising contacting a cell with the composition or pharmaceutical composition disclosed herein. In some embodiments, the present disclosure provides a method of modulating an immune response in a subject, the method comprising administering to the subject an effective amount of the composition or pharmaceutical composition disclosed herein. In some embodiments, the present disclosure provides a method of the subject has an auto-immune disorder or an inflammatory disorder. In some embodiments, the present disclosure provides a method of treating proliferative diseases or fibrotic disorders in a subject, the method comprising administering to the subject an effective amount of the composition or pharmaceutical composition disclosed herein.

In some embodiments, the present disclosure provides a method of treating cancer in a subject in need thereof, the method comprising administering to the subject an effective amount of the composition or pharmaceutical composition disclosed herein. In some embodiments, the cancer is a solid cancer or a blood cancer. In some embodiments, the solid cancer is a carcinoma or a sarcoma. In some embodiments, the solid cancer is kidney cancer, skin cancer, bladder cancer, bone cancer, brain cancer, breast cancer, colorectal cancer, esophageal cancer, eye cancer, head and neck cancer, lung cancer, ovarian cancer, pancreatic cancer, or prostate cancer. In some embodiments, the solid cancer is metastatic renal cell carcinoma (metastatic RCC) or melanoma. In some embodiments, the blood cancer is leukemia, Non-Hodgkin's lymphoma, Hodgkin's lymphoma, or multiple myeloma. In some embodiments, compositions described herein are used for the treatment of a cancer. In some embodiments, the cancer is a solid cancer. In some embodiments, the cancer is a blood cancer. In some embodiments, the solid cancer is a melanoma, lung, liver, head and neck, hepatocellular cancer, or pancreatic cancer. In some embodiments, the solid cancer is a hepatocellular cancer, melanoma, or lung cancer. In some embodiments, a composition described herein is used for reduction of a tumor size. In some embodiments, a composition described herein is used for reduction of a tumor volume. In some embodiments, a composition described herein is used for reduction of a cancer recurrence. In some embodiments, a composition described herein is used for reduction of tumor metastasis.

In some embodiments, a composition and/or pharmaceutical composition herein is used in a cell therapy or cell expansion application. In some embodiments, a composition or pharmaceutical composition described herein increases cell expansion in T-cells of a subject undergoing cell therapy: In some embodiments, cell expansion is increased while the T-cells still have a young cell phenotype. In some embodiments, a cell therapy is used for treatment of a disease, health condition, or wound described herein.

In some embodiments, the inflammatory response comprises cytokine production. In some embodiments, the inflammatory response comprises T cell production.

In some embodiments is provided a method of treating a subject in need thereof with a therapeutic effective dose of a composition or a pharmaceutical composition described herein. In some embodiments the therapeutic effective dose is a dose sufficient to induce an inflammatory response, to promote tumor reduction, or both. In some embodiments, a therapeutically effective amount is an amount sufficient to reduce, ameliorate, or prevent at least one symptom of a disease or condition.

In some embodiments, an engineered interleukin is independently present in an amount from: about 1 ng to about 100 ng, about 100 ng to about 500 ng, about 500 ng to about 1 mg, about 1 mg to about 500 mg, about 500 mg to about 1000 mg, about 1000 mg to about 5000 mg, about 5000 mg to about 10000 mg, about 10000 to 25000 mg, or about 25000 to 50000 mg, for example by itself or as part of a composition or pharmaceutical composition.

In some embodiments, contacting or applying to a tissue of, or administering to a patient or a patient in need thereof, occurs daily, every other day, every third day, every fourth day; every fifth day, every sixth day, weekly, every two weeks, every three weeks, once a month, once every three months, once every six months, once a year, or as needed. In some embodiments, the contacting is once, twice, three, four, five, six, seven, eight, nine, or ten times in a 24-hour period.

In some embodiments, a composition or pharmaceutical composition comprising an engineered cytokine, for example an engineered interleukin comprises a sequence of: any one of SEQ ID NO: 2-SEQ ID NO: 160, is contacted with, applied to, administered to, or contacts a population of cells once, twice, three, four, five, six, seven, eight, nine, or ten times in a 24-hour period, followed by a second composition or pharmaceutical composition independently comprising an engineered cytokine, for example an engineered interleukin comprising a sequence comprising any one of SEQ ID NO: 2-SEQ ID NO: 160, contacting a population of cells once, twice, three, four, five, six, seven, eight, nine, or ten times in a 24-hour period.

### Molecular Dynamics Simulations

Disclosed herein are methods of predicting a polypeptide structure using data input generated from molecular dynamics simulations. Molecular dynamics simulations can be performed in silico to model polypeptide structural conformations and biophysical features. Molecular dynamics simulations can allow for structural dynamics, such that the secondary and tertiary structure of a polypeptide can vary within the timeline of the simulation along allowed conformations. Generally, allowed conformations are those that represent minima along various free energy wells. As such, molecular dynamics simulations can be used to visualize and sample biologically relevant conformations that static structural techniques (e.g., x-ray crystallography) may not sample. Exemplary molecular dynamics simulations for inclusion in methods described herein include, without limitation, Classical Dynamics, Replica Exchange Molecular Dynamics, Meta-Dynamics, Langevin Dynamics, and Monte Carlo Dynamics.

Provided herein are methods wherein data generated from molecular dynamic simulations that is relied upon for modelling and predicting polypeptide structures. As described herein, data generated from molecular dynamics simulations is used as an input for machine learning to iterate among allowed and rare structural conformations to generate a more robust and fulsome predicted polypeptide structure. Such data can include residue-specific biophysical properties relevant to a single residue within the molecular dynamics simulation, as well as pairwise properties that relate to a set of biophysical properties relating to interactions between at least two residues within the molecular dynamics simulation. Examples of residue specific biophysical properties generated using molecular dynamics simulations include grand average of hydropathy (GRAVY) scores, a residue identity or label, coulombic energies, Van Der Waals energies, solvent accessible surface area (SASA), side chain order parameter (S2) and the like. Examples of pairwise biophysical properties generated using molecular dynamics simulations include distance between given residues, Coulombic energies, Van Der Waals energies, a fraction of native contacts (Q) and the like.

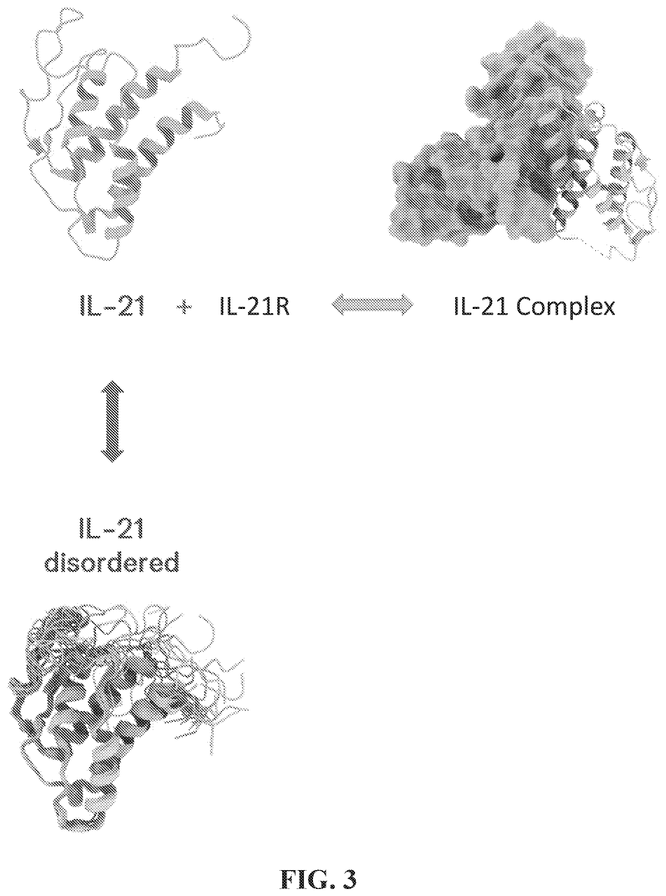

Such properties generated from molecular dynamics can be generated from a given conformation as a function of time. Accordingly, a data set of biophysical properties as a function of time from a set of polypeptide structures can be generated from the molecular dynamics simulations and used as input for machine learning algorithms. This data is arranged into a graph format prior to embedding. Each protein sequence of length L is mapped into undirected graph functions. FIG. 3 illustrates mapping of the individual graph functions as a function of time. Such graph functions can include:
continuous-time dynamic graph G(V, E_{T}, T) where V represents the set of nodes, E_{T}⊆V×V× [symbol] ⁺ represents the set of temporal edges between vertices in V and T is a function that maps each edge to a corresponding timestamp. Each edge eᵢ=(u, v, t)∈E_{T} is assigned to a unique time t∈ [symbol] ⁺ where (u, v) represents a couple of residues. This approach takes into account a set of time frames in the molecular dynamics simulation where, each time frame has a unique time t.
discrete-time dynamic graph G as a sequence of graphs G₁, G₂, . . . , G_{T} from timestamps 1 to T, where each timestamp represents a time frame in the molecular dynamics simulation. Each graph at time t is represented by Gₜ=(Vₜ, Eₜ) where Vₜ and Eₜ are the nodes and edges active between the timespan [tᵢ₋₁, tᵢ] of the graph respectively.
static graph G(V, E) where V represents the set of nodes and E represents the set of edges.

Each node in the static graph, continuous-time dynamic graph, and discrete time dynamic graph represents a residue while each edge represents the related pairwise residue-residue interaction, obtained from the compression of the information (e.g., arithmetic average) related to each time frame in the molecular dynamics simulation into a single time frame. In each graph function, ∥V∥=L, i.e., the number of nodes is equal to the sequence length L which can be different for each protein. Data generated from the dynamic graph representation is then encoded to be used as input for machine learning algorithms described herein. In some embodiments, a function ƒ: V→ [symbol] ^{D} that maps each vertex in either the continuous-time dynamic graph G(V, E_{T}, T) or the discrete-time dynamic graph Gₜ (Vₜ, Eₜ) into a D-dimensional vector is generated, where D is the embedding dimension.

A function can be a conditional log-probability of certain sets of temporal random walks. These are random walks that preserve the time order or the temporal edges, i.e. along a path of such a walk, the timestamp of the consecutive edges are non-decreasing. Additionally, such a function can be represented as a dynamic Skip-gram model trained on evolving random walks where, pre-trained Skip-gram model SGₜ₋₁ is used as initial weight for the next Skip-gram model SGₜ. Indeed, other such algorithms can be employed with the methods described herein.

After generation of graph representations as described herein, data is embedded for input into machine learning algorithms as described herein. In some instances, manifold learning techniques, e.g. t-distributed stochastic neighbor embedding (t-SNE), can be used. Embedding as described herein can include dynamic residue embedding and static protein embedding.

In dynamic residue embedding, each protein is mapped/embedded into a dense 2^{nd} rank tensor D_{μv}, where μ∈[0, . . . , ∥V∥] is the residue index and v∈[0, . . . , E_{d}] is the embedding index, where E_{d} is the dynamic embedding dimension. Hence each residue is embedded into a dense vector; rᵢ∈ [symbol] ^{E}^{_{d}} for i=0, . . . , ∥V∥ and a separate dynamic residue embedding is trained for each continuous-time dynamic graph or continuous-time dynamic graph, representing an element of C, the set of polypeptide structures. Stacking D^{μv} is derived from each protein sequence in C to generate D_{αμv} where α∈[0, . . . ∥C∥] is the protein index. rᵢ_{ⱼ} is the dynamic residue embedding vector where j∈[0, . . . , ∥C∥].

In static protein embedding, each protein is mapped/embedded into a dense 1^{st} rank tensor S^{rv}. v∈[0, . . . , Eₛ] is the embedding index, where Eₛ is the static embedding dimension, where the data output from dynamic graph embedding is used as an input. Hence each protein is embedded into a dense vector p∈ [symbol] ^{E}^{ₛ} and a single static protein embedding is trained taking into account every static graph, representing an element of C. Hausdorff distance d_{H}(D^{α=x,μ,v}, D^{α=y,μ,v}), as well as other types of distances such as Frobenius norm that involve the dynamic graph representation, can be used as graph proximity metric ∀x, y∈└0, . . . , ∥C∥┘. In some cases, the dynamic residue embedding tensors D^{α=x,μ,v} and D^{α=y,μ,v} can be two non-empty subsets of the metric space (R, d) where R represents the set of dynamic residue embedding vectors rᵢ_{ⱼ} ∀j∈[0, . . . , ∥C∥] and is the euclidean distance. Stacking S^{v} coming from each protein sequence in C is then generated to calculate S^{αv}.

### Machine Learning

Provided herein are methods wherein tensor representations D^{αμv} and S^{αv} generated from the dynamic and static embedding, respectively, are used as input for machine learning to iteratively generate low energy predicted polypeptide structures. Such machine learning framework can be used to shorten an effective simulation time, execute prediction tasks, and perform design related tasks, such that a more robust and fulsome polypeptide structure can be generated from the limited data obtained from the molecular dynamics simulations. The tensor representations D_{αμv} and S^{αv} generated from the dynamic and static embedding, respectively, allow for accurate prediction of structure beyond the current computational capabilities of molecular dynamics simulations.

In some embodiments, polypeptide structures can be generated using unstructured computation, artificial intelligence or deep learning. In some cases, unstructured computation can be employed such that calculations can be performed iteratively. Further, polypeptide structure calculation can rely on artificial intelligence or deep learning. For example, a method described herein such as random forest can employ deep learning to generate Gini impurity scores that can be used to parse out probes with improved predictive value.

In some embodiments, methods of structural prediction as described herein can employ machine learning and computational intelligence techniques, such as deep neural networks, and combinations of supervised, semi-supervised and unsupervised learning techniques. In some embodiments, methods of structural prediction as described herein employ a supervised algorithm (by way of non-limiting example, linear region, random forest classification, decision tree learning, ensemble learning, bootstrap aggregating, and the like). In some embodiments, methods of structural prediction as described herein employ a non-supervised algorithm (by way of non-limiting example, clustering or association).

In some embodiments, the methods of structural prediction as described herein may be configured to utilize one or more exemplary AI/machine learning techniques chosen from, but not limited to, decision trees, boosting, support-vector machines, neural networks, nearest neighbor algorithms, Naive Bayes, bagging, random forests, and the like. In some embodiments and, optionally, in combination of any embodiment described above or below; an exemplary neutral network technique may be one of, without limitation, feedforward neural network, radial basis function network, recurrent neural network, convolutional network (e.g., U-net) or other suitable network. In some embodiments and, optionally, in combination of any embodiment described above or below; an exemplary implementation of Neural Network may be executed as follows:
a) define Neural Network architecture/model,
b) transfer the input data to the exemplary neural network model,
c) train the exemplary model incrementally,
d) determine the accuracy for a specific number of timesteps,
c) apply the exemplary trained model to process the newly-received input data,
f) optionally and in parallel, continue to train the exemplary trained model with a predetermined periodicity:

In some embodiments and, optionally, in combination of any embodiment described above or below; the exemplary trained neural network model may specify a neural network by at least a neural network topology, a series of activation functions, and connection weights. For example, the topology of a neural network may include a configuration of nodes of the neural network and connections between such nodes. In some embodiments and, optionally, in combination of any embodiment described above or below; the exemplary trained neural network model may also be specified to include other parameters, including but not limited to, bias values/functions and/or aggregation functions. For example, an activation function of a node may be a step function, sine function, continuous or piecewise linear function, sigmoid function, hyperbolic tangent function, or other type of mathematical function that represents a threshold at which the node is activated. In some embodiments and, optionally, in combination of any embodiment described above or below, the exemplary aggregation function may be a mathematical function that combines (e.g., sum, product, etc.) input signals to the node. In some embodiments and, optionally, in combination of any embodiment described above or below; an output of the exemplary aggregation function may be used as input to the exemplary activation function. In some embodiments and, optionally, in combination of any embodiment described above or below; the bias may be a constant value or function that may be used by the aggregation function and/or the activation function to make the node more or less likely to be activated.

In some embodiments, the machine learning model for structural prediction processes the biophysical properties encoded in the embeddings described above by applying the parameters of the machine learning model to produce a model output. In some embodiments, the model output may be decoded to generate one or more numerical output values and/or vectors indicative of polypeptide structure.

In some embodiments, the parameters of the machine learning model may be trained based on known polypeptide structures. For example, the biophysical properties may be paired with a target structure and/or measurement to form a training pair, such as historical biophysical properties and an observed structure representing a data point in the relationship between the historical biophysical properties and structure. In some embodiments, the biophysical properties may be provided to the machine learning model, e.g., encoded in the embeddings, to produce data representative of polypeptide structure. In some embodiments, an optimization problem associated with the machine learning model may then compare the polypeptide structure with the known output of a training pair including the historical biophysical properties to determine an error of the polypeptide structure. In some embodiments, the optimization problem may employ a loss function, such as, e.g., Hinge Loss, Multi-class SVM Loss, Cross Entropy Loss, Negative Log Likelihood, or other suitable classification loss function to determine the error of the polypeptide structure based on the known structure.

In some embodiments, the known output may be obtained after the machine learning model produces the prediction, such as in online learning scenarios. In such a scenario, the machine learning model may receive the biophysical properties and generate the model output vector to produce the data representative of polypeptide structure. Subsequently; a user may provide feedback by, e.g., modifying, adjusting, removing, and/or verifying the predicted structure via a suitable feedback mechanism, such as a user interface device (e.g., keyboard, mouse, touch screen, user interface, or other interface mechanism of a user device or any suitable combination thereof). The feedback may be paired with the biophysical properties to form the training pair and the optimization problem may determine an error of the polypeptide structure using the feedback.

In some embodiments, based on the error, the optimization problem may update the parameters of the machine learning model using a suitable training algorithm such as, e.g., backpropagation for a prediction machine learning model. In some embodiments, backpropagation may include any suitable minimization algorithm such as a gradient method of the loss function with respect to the weights of the prediction machine learning model. Examples of suitable gradient methods include, e.g., stochastic gradient descent, batch gradient descent, mini-batch gradient descent, or other suitable gradient descent technique. As a result, the optimization problem may update the parameters of the machine learning model based on the error of predicted structure in order to train the machine learning model to model the correlation between biophysical properties and polypeptide structure in order to produce more accurate prediction of structure based on biophysical properties.

### Generation of Polypeptide Compositions

### Polypeptide and Polypeptide Structure Generation Using Predictive Data

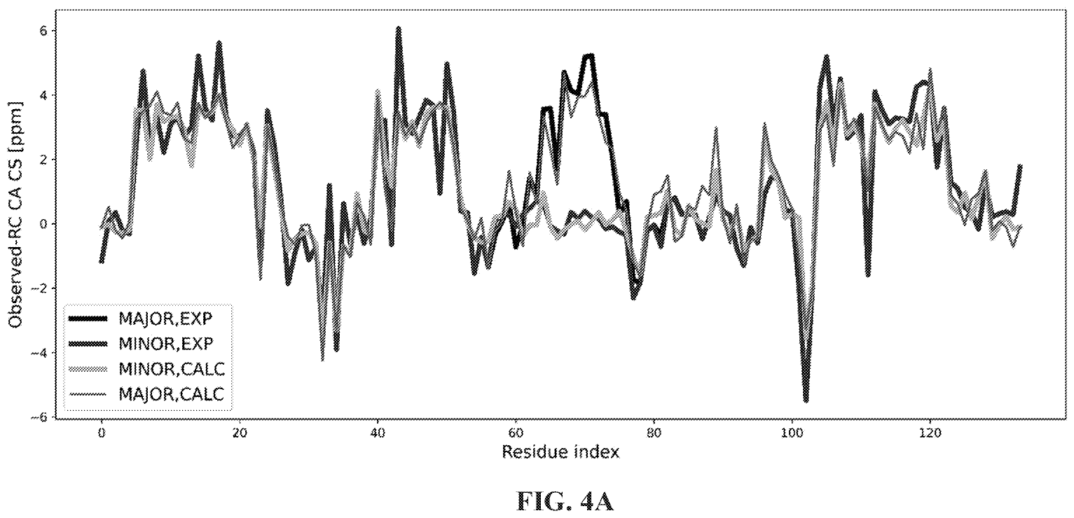

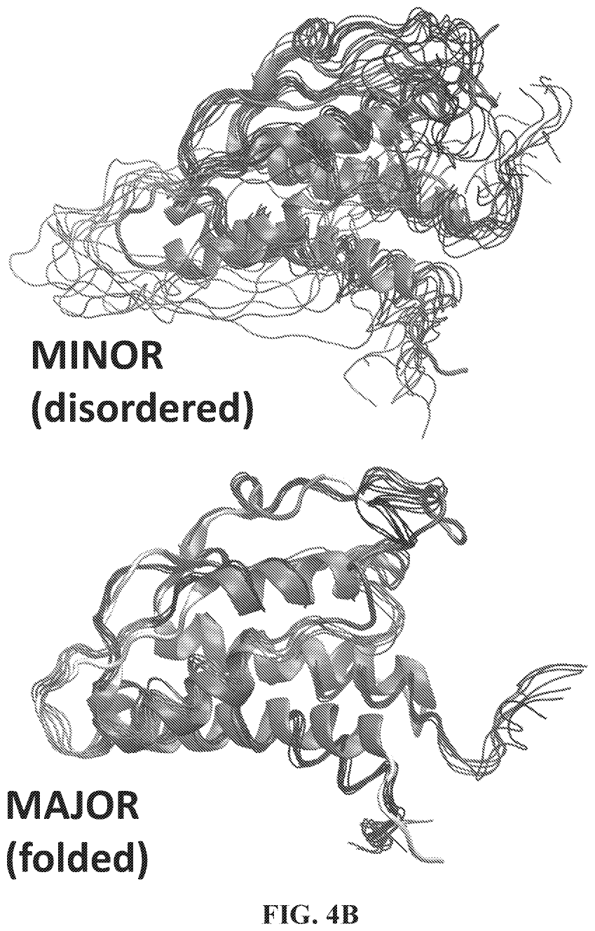

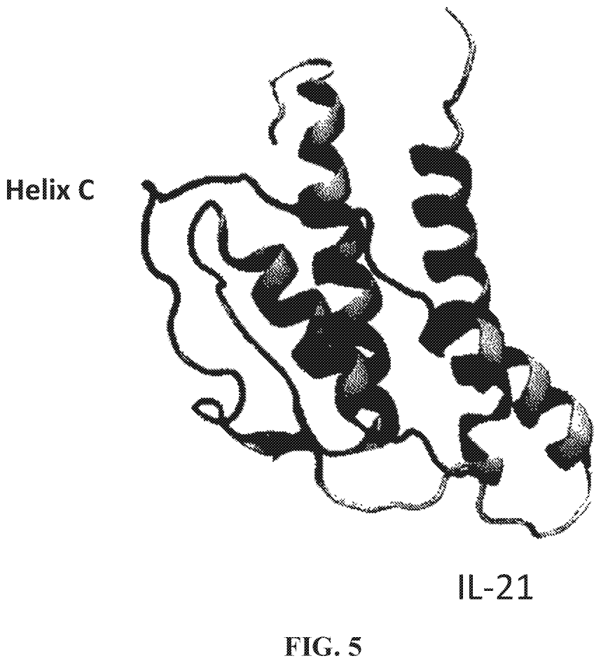

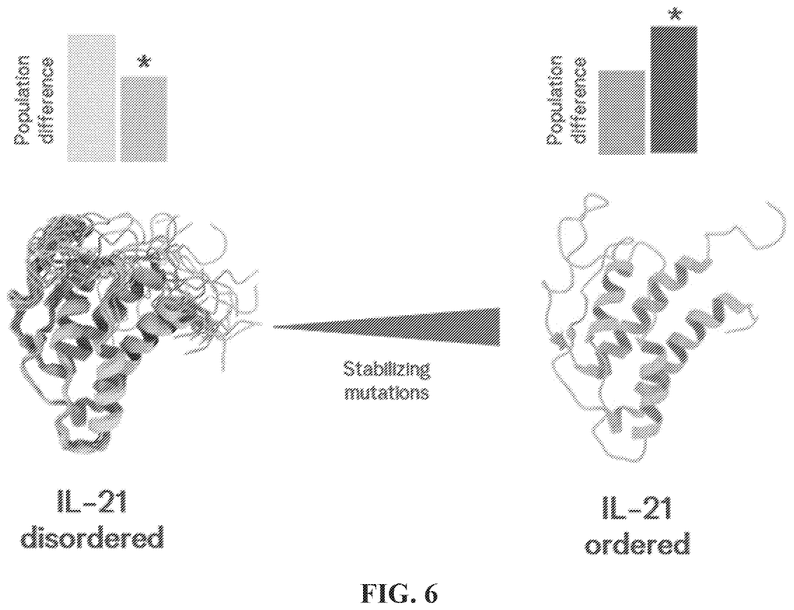

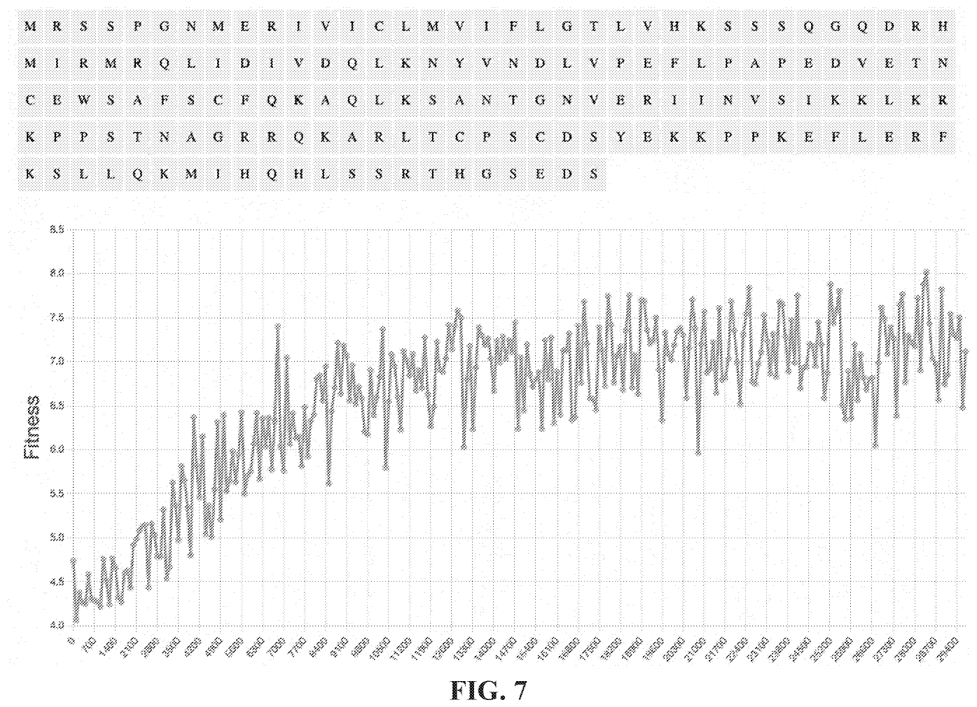

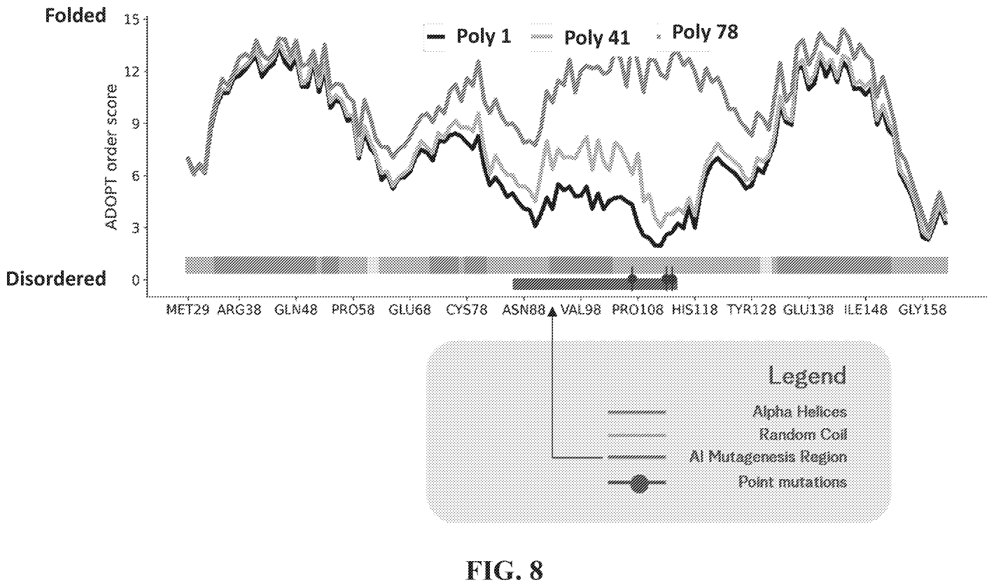

As described herein, robust and fulsome polypeptide structures can be predicted using data generated from molecular dynamics simulations using machine learning algorithms as described herein. Knowledge of such structures can be used to effectively and accurately map dynamic surfaces of a polypeptide of interest that is implicated in a disease or condition. By accurately modelling surfaces of polypeptide as a function of time, therapeutics can be generated that are able to bind to and interact with an epitope of the polypeptide of interest. Accordingly, such therapeutics can be generated with a paratope structure configured to bind to the epitope and are useful for treatment of diseases or condition. FIG. 4 depicts an illustration of a predicted epitope and paratope structure using methods described herein. Further, by capturing the dynamic structure of polypeptides using methods described herein, rare conformations that are biologically relevant can be predicted which may not be present in static structures such as those generated by x-ray crystallography. Further, iteration using machine learning using the methods described herein allows for robust simulation beyond the capability of molecular dynamics simulations alone, which allows for sampling of rare and short lived (though biologically relevant) conformations that produce epitopes.

Any combination of data can be utilized as described above to generate predicted polypeptide structures using any machine learning algorithm as described above. Further, additional input can be used to provide additional information useful for elucidating biologically relevant epitope conformations. For example, evolutionary covariance among related or homologous polypeptides can be used to determine conservation among residues separated in primary structure and secondary structure by significant distance. Without wishing to be bound by theory, methods described herein utilize evolutionary coupling between a pair of residues as an input to determine whether the pair of residues share a biological function (e.g., are present in the same binding epitope). With such input, dynamic modelling can be performed to determine whether such residues are present in the dynamic structure with minimal entropic penalty. Accordingly, evolutionary coupling and dynamics/disorder parameters are balanced to sample rare yet biologically relevant conformations that give rise to such epitopes.

Where evolutionary couplings are employed, the method described herein comprises generating multiple sequence alignments to determine homology among amino acid sequences. Identity between a reference sequence (query sequence, i.e., a sequence of the disclosure) and a subject sequence, also referred to as a global sequence alignment, can be determined using the FASTDB computer program based on the algorithm of Brutlag et al. (Comp. App. Biosci. 6:237-245 (1990)). In some embodiments, parameters using a FASTDB amino acid alignment, can include: Scoring Scheme-PAM (Percent Accepted Mutations) 0, k-tuple=2, Mismatch Penalty=1, Joining Penalty=20, Randomization Group Length=0, Cutoff Score=1, Window Size=sequence length, Gap Penalty-5, Gap Size Penalty=0.05, Window Size=500 or the length of the subject sequence, whichever can be shorter. According to this embodiment, if the subject sequence is shorter than the query sequence due to N- or C-terminal deletions, not because of internal deletions, a manual correction can be made to the results to take into consideration the fact that the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are lateral to the N- and C-terminal of the subject sequence, which is not matched/aligned with a corresponding subject residue, as a percent of the total bases of the query sequence. A determination of whether a residue is matched/aligned can be determined by results of the FASTDB sequence alignment. This percentage is then subtracted from the percent identity, calculated by the FASTDB program using the specified parameters, to arrive at a final percent identity score. This final percent identity score can be used for the purposes of this embodiment. In some cases, only residues to the N- and C-termini of the subject sequence, which is not matched/aligned with the query sequence, can be considered for the purposes of manually adjusting the percent identity score. That is, only query residue positions outside the farthest N- and C-terminal residues of the subject sequence may be considered for this manual correction. For example, a 90-residue subject sequence can be aligned with a 100-residue query sequence to determine percent identity. The deletion occurs at the N-terminus of the subject sequence, and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% can be subtracted from the percent identity score calculated by the FASTDB program. If the remaining 90 residues were perfectly matched, the final percent identity can be 90%. In another example, a 90-residue subject sequence can be compared with a 100-residue query sequence. This time the deletions can be internal deletions, so there can be no residues at the N-or C-termini of the subject sequence which can be not matched/aligned with the query. In this case, the percent identity calculated by FASTDB can be not manually corrected. Once again, only residue positions outside the N- and C-terminal ends of the subject sequence, as displayed in the FASTDB alignment, which can be not matched/aligned with the query sequence can be manually corrected for.

In some instances, a known structure can be utilized in conjunction with a sequence as an input for methods described herein. For example, a structure deposited in a protein structure database can be accessed and used as an input for determining novel epitopes. In some instances, empirical structural data can be used as an input. For example, a static structure of a target polypeptide obtained by X-ray crystallography can be used an input. Further, a dynamic structure obtained using techniques such as circular dichroism or NMR (e.g., 2D NMR, 3D NMR, solid-state NMR, and the like) can be used as an input.

An exemplary workflow of predicting an epitope structure follows below.
A protein sequence (or a list) is fed into the algorithm.
A multiple sequence alignment (MSA) is performed in order to evaluate evolutionary couplings (EC) between pairs of amino acid residues in the analyzed sequence. An evolutionary coupling reports on a probability that an arbitrary pair of amino acid residues in a given sequence evolved in a coupled fashion and thus is of evolutionary significance and likely has a biological role.
A protein homology 3D model (or models from protein sequence list) that resembles X-ray crystallography or NMR structure is computed.
A solvated 3D model of a protein (using SPC or TIP3 water models) is generated and the remaining, non-neutralized charges get neutralized by an addition of monovalent positive (Na+) and negative (Cl−) ions, so that the net charge of a simulated system (the sum of all charges) is equal to zero.
The solvated system is subjected to Replica Exchange Molecular Dynamics (REMD) simulation in which:
   a. An arbitrary number of simulation replicas (>2) is initiated. The number itself depends on the system size and scales up with the number of atoms, e.g., a 25000-atom system may require 25 replicas running for 500 nanoseconds each.
   b. Every replica receives a copy of an original forcefield assigned to the simulation, for which torsional angle potentials, dihedral potentials and selected non-bonded terms are scaled linearly by a factor proportional to the number of replicas. The first replica in the set receives full forces, whereas the last replica is exposed to a engineered forcefield scaled by an effective factor equal to 0.5.
Upon the execution of REMD, a Free Energy Surface (FES) of a configurational protein space is reconstructed, so that the most representative structures belonging to different free energy wells can be identified and bundled together as a 3D protein ensemble.
A newly constructed 3D protein ensemble is a subject to a sub-domain identification procedure, which evaluates geometrical and spatio-temporal suitability of a target protein fragments using the following metrics:
   a. Structural disorder of individual protein fragments from:
      i. Protein backbone H—N bond order parameters (S2).
      ii. Root Mean Square Fluctuations (RMSD) of CA atoms in protein backbone.
   b. Structural prominence from:
      i. Solvent Accessible Surface (SASA) of exposed amino acids.
      ii. Atomic volume map (AVM).
A graph network is constructed, in which every CA atom in the original 3D protein molecule is represented by a node, whereas its interactions with neighboring CA backbone atoms are represented by graph edges. In this representation:
   a. graph nodes are assigned:
      i. RMSF and S2.
      ii. the sum of intra-residue interaction energies computed from REMD protocol.
      iii. Combined SASA and AVM.
   b. graph edges are assigned:
      i. intra-residual interaction energies estimated from REMD protocol.
      ii. EC probabilities derived from step 2 of the algorithm.
Graph nodes clustering algorithms are applied to graphs from step 8, so that clusters of amino acid residues that share similar spatio-temporal (dynamics) and structural prominence can be identified and flagged as sub-domains. The clustering algorithms may include:
   a. K-means clustering.
   b. t-distributed stochastic neighbor embedding (t-SNE)
   c. and equivalent.
A composite druggability index (DI) is devised and computed for all clustered classes. The score is a sum of structural prominence from SASA and AVM, evolutionary conservation from EC, divided by a sum of RMSF and an inverse of S2. High score indicates domains that are prominent, exposed to solvent yet undergo small structural transitions throughout their molecular dynamics. Moreover, an addition of EC components allows for prioritization of sites with strongly conserved evolutionary features. Low scores denote domains of poor structural prominence, high dynamics and importantly low evolutionary conservation.
The DI score can be further enhanced by an addition of manually curated data on antibody-epitope interactions, such as IC₅₀ binding values. Such data can originate from privately performed experiments or through an automated literature search using Natural Language Processing (NLP) methods.

Provided herein are methods wherein after prediction of an epitope surface using methods described herein, a protein therapeutic is designed in silico to comprise a paratope structure that is configured to bind to and interact with the predicted epitope structure. A protein therapeutic can be synthesized using standard FMOC protein synthesis or other standard peptide synthesis techniques used in the art. Alternatively, some protein therapeutics can be expressed in a microorganism such as Escherichia coli from a DNA vector. In such embodiments, a polynucleotide sequence encoding the polypeptide of interest is subcloned into an expression vector for overexpression in the microorganism. Successful subcloning of the polynucleotide sequence can be confirmed by sequencing using commercially readily available methods including, without limitation, capillary sequencing, bisulfite-free sequencing, bisulfite sequencing. TET-assisted bisulfite (TAB) sequencing, ACE-sequencing, high-throughput sequencing, Maxam-Gilbert sequencing, massively parallel signature sequencing, Polony sequencing, 454 pyrosequencing, Sanger sequencing, Illumina sequencing, SOLID sequencing, Ion Torrent semiconductor sequencing. DNA nanoball sequencing, Heliscope single molecule sequencing, single molecule real time (SMRT) sequencing, nanopore sequencing, shot gun sequencing, RNA sequencing, Enigma sequencing, or any combination thereof.

Such protein therapeutics contain high potency of binding for the predicted epitope based on the robust structural sampling methods provided herein. Accordingly; such therapeutic polypeptides display biologically relevant activity against the protein of interest when administered to the subject.

Further, such protein therapeutics are expected to have high specificity and selectivity against the protein of interest. In some cases, a protein of interest can have a specificity of at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% for the target of interest, as determined for example in an in vitro competitive assay. In some cases, a protein of interest can have a selectivity of at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% for the target of interest among other proteins, as determined for example in an in vitro competitive assay.

### Systems

Also disclosed herein are systems for performing methods described herein. A system can comprise a computer readable memory storing instructions for performing methods described herein. For example, the computer readable memory can comprise instructions for in silico determination of polypeptide structure as described herein. In some embodiments, the computer readable memory can comprise instructions for epitope determination as described herein.

A system can further comprise computer systems utilizing the computer readable memory. Computer systems can include a processor operatively coupled to the computer readable memory; and can be configured to execute the instructions to perform a method described herein. A computer system can further include user input and output means, such as a keyboard, monitor, and mouse.

A system as described herein can be configured to access a database. For example, a system can be configured to access local or online (e.g., cloud) databases such as protein structure database, protein sequence databases, homology databases, nucleic acid sequence databases, and the like.

Upon execution of a method described herein, a system can further comprise data obtained by executing a method described herein. For example, a system upon execution of a method described herein can comprise druggability index scores for determining novel epitopes. Example 5 herein provides an exemplary output of such data that can be stored on a system after execution of a method described herein. A system can comprise structural information obtained from MD simulations described herein. Further, a system can comprise empirical structural data such as protein structures obtained from NMR, mass spectrometry, X-ray crystallography, or a combination thereof. A system can comprise an optimized polypeptide structure obtained using the in silico methods described herein.

Such systems can include storage means for storing or transferring data obtained by the methods described herein. In some instances, the systems can include means to transmit data obtained by the methods described herein into an external database (e.g., a local database or an online database).

### EXEMPLARY EMBODIMENTS

Provided herein are compositions comprising: an engineered IL-21 cytokine or a functional fragment thereof, that is a variant of a wild type IL-21 cytokine having amino acid residues of SEQ ID NO: 1, wherein the engineered IL-21 cytokine or a functional fragment thereof includes at least one amino acid substitution in a region including amino acid residues 30 to 135 of SEQ ID NO: 1, wherein the at least one amino acid substitution provides for an increased stability of the engineered IL-21 cytokine compared to the wild type IL-21 cytokine, and wherein the increased stability is characterized by an increased thermal stability of the engineered IL-21 cytokine compared to that of the wild type IL-21 cytokine when measured in a 75 ug/mL sample using a differential scanning fluorimetry (DSF) spectroscopic method to detect thermal melting measurements by performing a thermal ramp with a 1 degree Celsius/minute heating rate from 20 degrees Celsius to 95 degrees Celsius and measuring backscattered light intensity to detect heat induced aggregation. Also provided herein are compositions, wherein the engineered IL-21 cytokine or a functional fragment thereof includes an amino acid sequence having at least 80%, at least 85%, at least 90%, or at least 95% sequence identity to SEQ ID NO: 81. Also provided herein are compositions, wherein the engineered IL-21 cytokine or a functional fragment thereof includes at least two amino acid substitutions, at least three substitutions, at least four amino acid substitutions, or at least five amino acid substitutions in a region including amino acid residues of SEQ ID NO: 81. Also provided herein are compositions, wherein the engineered IL-21 cytokine or a functional variant thereof includes an amino acid sequence selected from any one of SEQ ID NO: 2 to SEQ ID NO: 160. Also provided herein are compositions, wherein the engineered IL-21 cytokine or a functional variant thereof includes the at least one amino acid substitution, at least two amino acid substitutions, at least three substitutions, at least four amino acid substitutions or at least five amino acid substitutions in a region including amino acid residues 30-135 of SEQ ID NO: 1 is at positions selected from: R34, H35, 137, R38, D44, 145, Q48, N70, A82, T89, G90, N92, E93, 195, 196, V98, K102, L103, K104, R105, P107, T110, N111, A112, G113, R114, R115, Q116, H118, R119, L120, or P133.

Also provided herein are compositions, wherein the at least one amino acid substitution in a region including amino acid residues 30-135 of SEQ ID NO: 1 is at position R34. Also provided herein are compositions, wherein the at least one amino acid substitution in a region including amino acid residues 30-135 of SEQ ID NO: 1 is at position 137. Also provided herein are compositions, wherein the at least one amino acid substitution in a region including amino acid residues 30-135 of SEQ ID NO: 1 is at position R38. Also provided herein are compositions, wherein the at least one amino acid substitution in a region including amino acid residues 30-135 of SEQ ID NO: 1 is at position D44. Also provided herein are compositions, wherein the at least one amino acid substitution in a region including amino acid residues 30-135 of SEQ ID NO: 1 is at position 145. Also provided herein are compositions, wherein the at least one amino acid substitution in a region including amino acid residues 30-135 of SEQ ID NO: 1 is at position Q48. Also provided herein are compositions, wherein the at least one amino acid substitution in a region including amino acid residues 30-135 of SEQ ID NO: 1 is at position N70. Also provided herein are compositions, wherein the at least one amino acid substitution in a region including amino acid residues 30-135 of SEQ ID NO: 1 is at position A82. Also provided herein are compositions, wherein the at least one amino acid substitution in a region including amino acid residues 30-135 of SEQ ID NO: 1 is at position T89. Also provided herein are compositions, wherein the at least one amino acid substitution in a region including amino acid residues 30-135 of SEQ ID NO: 1 is at position G90. Also provided herein are compositions, wherein the at least one amino acid substitution in a region including amino acid residues 30-135 of SEQ ID NO: 1 is at position N92. Also provided herein are compositions, wherein the at least one amino acid substitution in a region including amino acid residues 30-135 of SEQ ID NO: 1 is at position E93. Also provided herein are compositions, wherein the at least one amino acid substitution in a region including amino acid residues 30-135 of SEQ ID NO: 1 is at position I95. Also provided herein are compositions, wherein the at least one amino acid substitution in a region including amino acid residues 30-135 of SEQ ID NO: 1 is at position I96. Also provided herein are compositions, wherein the at least one amino acid substitution in a region including amino acid residues 30-135 of SEQ ID NO: 1 is at position V98. Also provided herein are compositions, wherein the at least one amino acid substitution in a region including amino acid residues 30-135 of SEQ ID NO: 1 is at position K102. Also provided herein are compositions, wherein the at least one amino acid substitution in a region including amino acid residues 30-135 of SEQ ID NO: 1 is at position L103. Also provided herein are compositions, wherein the at least one amino acid substitution in a region including amino acid residues 30-135 of SEQ ID NO: 1 is at position K104. Also provided herein are compositions, wherein the at least one amino acid substitution in a region including amino acid residues 30-135 of SEQ ID NO: 1 is at position R105. Also provided herein are compositions, wherein the at least one amino acid substitution in a region including amino acid residues 30-135 of SEQ ID NO: 1 is at position P107. Also provided herein are compositions, wherein the at least one amino acid substitution in a region including amino acid residues 30-135 of SEQ ID NO: 1 is at position T110. Also provided herein are compositions, wherein the at least one amino acid substitution in a region including amino acid residues 30-135 of SEQ ID NO: 1 is at position N111. Also provided herein are compositions, wherein the at least one amino acid substitution in a region including amino acid residues 30-135 of SEQ ID NO: 1 is at position A112. Also provided herein are compositions, wherein the at least one amino acid substitution in a region including amino acid residues 30-135 of SEQ ID NO: 1 is at position G113. Also provided herein are compositions, wherein the at least one amino acid substitution in a region including amino acid residues 30-135 of SEQ ID NO: 1 is at position R114. Also provided herein are compositions, wherein the at least one amino acid substitution in a region including amino acid residues 30-135 of SEQ ID NO: 1 is at position R115. Also provided herein are compositions, wherein the at least one amino acid substitution in a region including amino acid residues 30-135 of SEQ ID NO: 1 is at position Q116. Also provided herein are compositions, wherein the at least one amino acid substitution in a region including amino acid residues 30-135 of SEQ ID NO: 1 is at position H118. Also provided herein are compositions, wherein the at least one amino acid substitution in a region including amino acid residues 30-135 of SEQ ID NO: 1 is at position R119. Also provided herein are compositions, wherein the at least one amino acid substitution in a region including amino acid residues 30-135 of SEQ ID NO: 1 is at position L120. Also provided herein are compositions, wherein the at least one amino acid substitution in a region including amino acid residues 30-135 of SEQ ID NO: 1 is at position P133. Also provided herein are compositions, wherein the at least two amino acid substitutions in a region including amino acid residues 30-135 of SEQ ID NO: 1 are at positions R105 and P107. Also provided herein are compositions, wherein the at least two amino acid substitutions in a region including amino acid residues 30-135 of SEQ ID NO: 1 are at positions R38 and P107. Also provided herein are compositions, wherein the at least three amino acid substitutions in a region including amino acid residues 30-135 of SEQ ID NO: 1 are at positions E93, K104, and P107. Also provided herein are compositions, wherein the at least three amino acid substitutions in a region including amino acid residues 30-135 of SEQ ID NO: 1 are at positions N92, P107, and T110. Also provided herein are compositions, wherein the at least three amino acid substitutions in a region including amino acid residues 30-135 of SEQ ID NO: 1 are at positions N92, G113, and Q116. Also provided herein are compositions, wherein the at least three amino acid substitutions in a region including amino acid residues 30-135 of SEQ ID NO: 1 are at positions T89, N92, and P133. Also provided herein are compositions, wherein the at least three amino acid substitutions in a region including amino acid residues 30-135 of SEQ ID NO: 1 are at positions A82, 196, and L103. Also provided herein are compositions, wherein the at least three amino acid substitutions in a region including amino acid residues 30-135 of SEQ ID NO: 1 are at positions P107, G113, and R114. Also provided herein are compositions, wherein the at least three amino acid substitutions in a region including amino acid residues 30-135 of SEQ ID NO: 1 are at positions R38, R105, and P107. Also provided herein are compositions, wherein the at least five amino acid substitutions in a region including amino acid residues 30-135 of SEQ ID NO: 1 are at positions N92, E93, P107, N111, and Q116. Also provided herein are compositions, wherein the at least five amino acid substitutions in a region including amino acid residues 30-135 of SEQ ID NO: 1 are at positions H35, E93, K104, P107, and N111. Also provided herein are compositions, wherein the at least one amino acid substitution of SEQ ID NO: 1 at position R34 is R34P or R34W. Also provided herein are compositions, wherein the at least one amino acid substitution of SEQ ID NO: 1 at position H35 is H35C, H35F. H35W, H35Y, H35G, or H35P. Also provided herein are compositions, wherein the at least one amino acid substitution of SEQ ID NO: 1 at position 137 is I37K, 137R, or I37H. Also provided herein are compositions, wherein the at least one amino acid substitution of SEQ ID NO: 1 at position R38 is R38F, R38W, or R38E. Also provided herein are compositions, wherein the at least one amino acid substitution of SEQ ID NO: 1 at position D44 is D44K or D44H. Also provided herein are compositions, wherein the at least one amino acid substitution of SEQ ID NO: 1 at position 145 is 145V or 145. In some embodiments, the at least one amino acid substitution of SEQ ID NO: 1 at position Q48 is Q48T or Q48S. Also provided herein are compositions, wherein the at least one amino acid substitution of SEQ ID NO: 1 at position N70 is N70G. Also provided herein are compositions, wherein the at least one amino acid substitution of SEQ ID NO: 1 at position A82 is A82I, A82M, A82A, A82P, A82Y, A82W, A82M, or A82R. Also provided herein are compositions, wherein the at least one amino acid substitution of SEQ ID NO: 1 at position T89 is T89L, T89I, T89A, T89P, T89Y, or T89W. Also provided herein are compositions, wherein the at least one amino acid substitution of SEQ ID NO: 1 at position G90 is G90Y or G90W. Also provided herein are compositions, wherein the at least one amino acid substitution of SEQ ID NO: 1 at position N92 is N92M, N92L, N92I, N92V, N92A, N92P, N92Y. or N92W. Also provided herein are compositions, wherein the at least one amino acid substitution of SEQ ID NO: 1 at position E93 is E93K, E93H, E93R, or E93L. Also provided herein are compositions, wherein the at least one amino acid substitution of SEQ ID NO: 1 at position I95 is I95K, I95L, or I95R. Also provided herein are compositions, wherein the at least one amino acid substitution of SEQ ID NO: 1 at position I96 is I96P, I96L, I96I, I96A, I96Y, I96W, or I96M. Also provided herein are compositions, wherein the at least one amino acid substitution of SEQ ID NO: 1 at position V98 is V98W or V98Y. Also provided herein are compositions, wherein the at least one amino acid substitution of SEQ ID NO: 1 at position K102 is K102T, K102Y, or K102F. Also provided herein are compositions, wherein the at least one amino acid substitution of SEQ ID NO: 1 at position L103 is L103P, L103L, L103I, L103A, or L103Y. Also provided herein are compositions, wherein the at least one amino acid substitution of SEQ ID NO: 1 at position K104 is K104L, K104A, K104P, K104Y, K104W, or K104M. Also provided herein are compositions, wherein the at least one amino acid substitution of SEQ ID NO: 1 at position R105 is R105W. Also provided herein are compositions, wherein the at least one amino acid substitution of SEQ ID NO: 1 at position P107 is P107L, P107I, P107A, P107P, P107Y, P107W, P107M, or P107N. Also provided herein are compositions, wherein the at least one amino acid substitution of SEQ ID NO: 1 at position T110 is T110L, T110I, T110A, T110P, T110Y, T110W, or T110M. Also provided herein are compositions, wherein the at least one amino acid substitution of SEQ ID NO: 1 at position N111 is N111E, N111K, N111H, N111R, N111L, N111W, N111I, or N111M. Also provided herein are compositions, wherein the at least one amino acid substitution of SEQ ID NO: 1 at position A112 is A112I, A112M, or A112W. Also provided herein are compositions, wherein the at least one amino acid substitution of SEQ ID NO: 1 at position G113 is G113L. G113I, G113A, G113P, G113Y, G113W, G113M, or G113Q. Also provided herein are compositions, wherein the at least one amino acid substitution of SEQ ID NO: 1 at position R114 is R114L, R114I, R114A, R114P, R114Y, R114W, or R114M. Also provided herein are compositions, wherein the at least one amino acid substitution of SEQ ID NO: 1 at position R115 is R115F or R115Y. Also provided herein are compositions, wherein the at least one amino acid substitution of SEQ ID NO: 1 at position Q116 is Q116L, Q116I, Q116A, Q116P, Q116Y, Q116W, or Q116M. Also provided herein are compositions, wherein the at least one amino acid substitution of SEQ ID NO: 1 at position H118 is H118P. Also provided herein are compositions, wherein the at least one amino acid substitution of SEQ ID NO: 1 at position R119 is R119M or R119P. Also provided herein are compositions, wherein the at least one amino acid substitution of SEQ ID NO: 1 at position L120 is L120Y. Also provided herein are compositions, wherein the at least one amino acid substitution of SEQ ID NO: 1 at position P133 is P133L, P133I, P133A, P133P, P133Y, P133W, or P133M. Also provided herein are compositions, wherein the at least one amino acid substitution in a region including amino acid residues 30-135 of SEQ ID NO: 1 is A82I. Also provided herein are compositions, wherein the at least one amino acid substitution in a region including amino acid residues 30-135 of SEQ ID NO: 1 is N92M. Also provided herein are compositions, wherein the at least one amino acid substitution in a region including amino acid residues 30-135 of SEQ ID NO: 1 is P107L. Also provided herein are compositions, wherein the at least one amino acid substitution in a region including amino acid residues 30-135 of SEQ ID NO: 1 include two amino acid substitutions. Also provided herein are compositions, wherein the two amino acid substitutions are R105W and P107L. Also provided herein are compositions, wherein the two amino acid substitutions are R38F and P107L. Also provided herein are compositions, wherein the two amino acid substitutions are R38W and P107L. Also provided herein are compositions, wherein the two amino acid substitutions are R38E and P107L. Also provided herein are compositions, wherein the at least one amino acid substitution in a region including amino acid residues 30-135 of SEQ ID NO: 1 include three amino acid substitutions. Also provided herein are compositions, wherein the three amino acid substitutions are E93L, K104L, and P107L. Also provided herein are compositions, wherein the three amino acid substitutions are N92L, P107I, and T110L. Also provided herein are compositions, wherein the three amino acid substitutions are N92I, G113L, and Q116L. Also provided herein are compositions, wherein the three amino acid substitutions are T89L, N92M, and P133L. Also provided herein are compositions, wherein the three amino acid substitutions are A82R, 196P, and L103P. Also provided herein are compositions, wherein the three amino acid substitutions are P107W, G113Y, and R114L. Also provided herein are compositions, wherein the three amino acid substitutions are R38F, R105W, and P107L. Also provided herein are compositions, wherein the three amino acid substitutions are R38W, R105W, and P107L. Also provided herein are compositions, wherein the at least one amino acid substitution in a region including amino acid residues 30-135 of SEQ ID NO: 1 include five amino acid substitutions. Also provided herein are compositions, wherein the five amino acid substitutions are N92I, E93L, P107N, N111E, and Q116L. Also provided herein are compositions, wherein the five amino acid substitutions are H35C, E93L, K104L, P107L, and N111L. Also provided herein are compositions, wherein the variant of the wild type IL-21 cytokine includes a deletion, a substitution, or an addition in the region including amino acid residues of 30-135 SEQ ID NO: 1.

Also provided herein are compositions, wherein the composition of the present disclosure provides for compositions wherein the DSF spectroscopic method determines a melting temperature of the engineered IL-21 cytokine or the wild type IL-21 cytokine, wherein the melting temperature of the engineered IL-21 cytokine is increased by about 2 degrees Celsius, about 3 degrees Celsius, about 4 degrees Celsius, about 5 degrees Celsius, about 6 degrees Celsius, about 7 degrees Celsius, about 8 degrees Celsius, about 9 degrees Celsius, about 10 degrees Celsius, about 11 degrees Celsius, about 12 degrees Celsius, about 13 degrees Celsius, about 14 degrees Celsius, or about 15 degrees Celsius, compared to that of the wild type IL-21 cytokine. Also provided herein are compositions, wherein the melting temperature of the engineered IL-21 cytokine is increased by about 7 degrees Celsius compared to that of the wild type IL-21 cytokine. Also provided herein are compositions, wherein the melting temperature of the engineered IL-21 cytokine is increased by about 11 degrees Celsius compared to that of the wild type IL-21 cytokine.

Also provided herein are compositions, wherein the increased stability is further characterized by an increased resistance to pepsin digestion of the engineered IL-21 cytokine compared to that of the wild type IL-21 cytokine, wherein the pepsin digestion produces peptide fragments, and wherein the increased resistance to pepsin digestion is evaluated by quantifying the peptide fragments measured by mass spectrometry. Also provided herein are compositions, wherein the increased resistance is about 10%, about 12%, about 14%, about 16%, about 18%, about 20%, or about 22%. Also provided herein are compositions, wherein the increased resistance is about 12%. Also provided herein are compositions, wherein the increased resistance is about 20%.

Also provided herein are compositions, wherein the increased stability is further characterized by an increased structural rigidity of the engineered IL-21 cytokine compared to the wild type IL-21 cytokine. Also provided herein are compositions, wherein the increased structural rigidity is measured by an increased proton-deuterium exchange rate of the engineered IL-21 cytokine compared to that of the wild type IL-21 cytokine when measured by hydrogen deuterium exchange mass spectrometry (HDX-MS).

Also provided herein are compositions, wherein the increased stability is further characterized by an increased compactness of the engineered IL-21 cytokine compared to that of the wild type IL-21 cytokine as measured by dynamic light scattering (DLS). Also provided herein are compositions, wherein the DLS determines an average particle diameter of the engineered IL-21 cytokine or the wild type IL-21 cytokine, wherein the average particle diameter of the engineered IL-21 cytokine is decreased by about 0.10 nm, about 0.15 nm, about 0.20 nm, or about 0.25 nm compared to that of the wild type IL-21 cytokine. Also provided herein are compositions, wherein the average particle diameter of the engineered IL-21 cytokine is decreased by about 0.20 nm compared to that of the wild type IL-21 cytokine. Also provided herein are compositions, wherein the average particle diameter of the engineered IL-21 cytokine is decreased by about 0.25 nm compared to that of the wild type IL-21 cytokine.

Also provided herein are compositions, wherein the increased stability is further characterized by an increased yield in an expression system of the engineered IL-21 cytokine compared to that of the wild type IL-21 cytokine. Also provided herein are compositions, wherein the increased yield is about 2-fold, about 3-fold, about 4-fold, about 5-fold, about 6-fold, about 7-fold, about 8-fold, about 9-fold, about-10 fold, about 11-fold, about 12-fold, about 13-fold, about 14-fold, or about 15-fold. Also provided herein are compositions, wherein the increased yield is about 4-fold. Also provided herein are compositions, wherein the increased yield is about 11-fold. Also provided herein are compositions, wherein the increased yield is about 13-fold.

Also provided herein are compositions, wherein the engineered IL-21 cytokine induces a higher STAT3 phosphorylation in a cell line compared to that of the wild type IL-21 cytokine.

Also provided herein are compositions, wherein the engineered cytokine has a lower affinity to an IL-21 receptor compared to that of the wild type IL-21 cytokine. Also provided herein are compositions, wherein the lower affinity to the IL-21 receptor is lower by about 80-fold, about 90-fold, about 100-fold, about 110-fold, about 120-fold, or about 130-fold. Also provided herein are compositions, wherein the lower affinity to the IL-21 receptor is lower by about 100-fold. Also provided herein are compositions, wherein the lower affinity to the IL-21 receptor is lower by about 110-fold.

Also provided herein are compositions, the engineered IL-21 cytokine results in an improved exposure following administration to a subject relative to the wild type IL-21 cytokine, as measured by a greater area under curve (AUC) for the engineered IL-21 cytokine.

Provided herein are compositions comprising engineered cytokines that are variants of a wild type cytokine including a tertiary structure with 4 alpha helices, wherein the engineered cytokines includes at least one amino acid substitution in a non-alpha helical coil region compared to the wild type cytokine that provides for an increased stability of the engineered cytokines compared to the wild type cytokine, wherein the increased stability is characterized by an increased thermal stability of the engineered cytokine compared to that of the wild type cytokine when measured in a 75 μg/mL sample using a differential scanning fluorimetry (DSF) spectroscopic method to detect thermal melting measurements by performing a thermal ramp with a 1 degree Celsius/minute heating rate from 20 degrees Celsius to 95 degrees Celsius and measuring backscattered light intensity to detect heat induced aggregation. Also provided herein are compositions, wherein the engineered cytokines are engineered IL-2 cytokines, engineered IL-4 cytokines, engineered IL-7 cytokines, engineered IL-9 cytokines, engineered IL-15 cytokines, or engineered IL-21 cytokines.

The present disclosure provides compositions comprising: an engineered cytokine that is a variant of a wild type cytokine including a disordered region, wherein the engineered cytokine includes at least one amino acid substitution in the disordered region, wherein the at least one amino acid substitution provides for increased stability of the engineered cytokine compared to the wild type cytokine, and wherein the increased stability is characterized by an increased thermal stability of the engineered cytokine compared to the wild type cytokine when measured in a 75 μg/mL sample using a differential scanning fluorimetry (DSF) spectroscopic method to detect thermal melting measurements by performing a thermal ramp with a 1 degree Celsius/minute heating rate from 20 degrees Celsius to 95 degrees Celsius and measuring backscattered light intensity to detect heat induced aggregation. Also provided herein are compositions, wherein the disordered region lacks a stable tertiary structure. Also provided herein are compositions, wherein the disordered region includes at least one alpha-helical conformation component. Also provided herein are compositions, wherein the engineered cytokine further includes at least one amino acid substitution in a region that is not the disordered region. Also provided herein are compositions, wherein the engineered cytokine includes 4 alpha helices. Also provided herein are compositions, wherein the wild type cytokine is a human cytokine.

Provided herein are pharmaceutical compositions comprising the compositions disclosed herein. Also provided herein are pharmaceutical compositions, wherein the pharmaceutical compositions, further include solubilizing agents and excipients. Also provided herein are pharmaceutical compositions, wherein the excipients include one or more of a buffering agent, a stabilizer, an antioxidant, a binder, a diluent, a dispersing agent, a rate controlling agent, a lubricant, a glidant, a disintegrant, a plasticizer, a preservative, or any combinations thereof. Also provided herein are pharmaceutical compositions, wherein the pharmaceutical compositions are formulated for parenteral or enteral administration. Also provided herein are pharmaceutical compositions, wherein pharmaceutical compositions are in a lyophilized form.

Also provided herein are methods of inducing cell death, the methods comprising contacting a cell with the composition or pharmaceutical composition disclosed herein. Also provided herein are methods, wherein the cell is selected from: a lymphocyte cell, a B lymphocyte cell, or an MC116 cell.

Also provided herein are methods of reducing cancer cell growth, the methods comprising contacting a cell with the composition or pharmaceutical composition disclosed herein.

Also provided herein are methods of modulating an immune response in a subject, the methods comprising administering to the subject an effective amount of the composition or pharmaceutical composition disclosed herein.

Also provided herein are methods of treating proliferative diseases or fibrotic disorders in a subject, the methods comprising administering to the subject an effective amount of the composition or pharmaceutical composition disclosed herein.

Also provided herein are methods of treating cancer in a subject in need thereof, the methods comprising administering to the subject an effective amount of the composition or pharmaceutical composition disclosed herein. Also provided herein are methods, wherein the cancer is a solid cancer or a blood cancer. Also provided herein are methods, wherein the solid cancer is a carcinoma or a sarcoma. Also provided herein are compositions, wherein the solid cancer is kidney cancer, skin cancer, bladder cancer, bone cancer, brain cancer, breast cancer, colorectal cancer, esophageal cancer, eye cancer, head and neck cancer, lung cancer, ovarian cancer, pancreatic cancer, or prostate cancer. Also provided herein are methods, wherein the solid cancer is metastatic renal cell carcinoma (metastatic RCC) or melanoma. Also provided herein are methods, wherein the blood cancer is leukemia, Non-Hodgkin's lymphoma, Hodgkin's lymphoma, or multiple myeloma.

Provided herein are polynucleotides encoding the engineered IL-21 cytokines or a functional fragment thereof described herein or the engineered cytokines described herein. Also provided herein are polynucleotides, wherein the polynucleotides comprise a nucleotide sequence encoding the engineered IL-21 cytokines or a functional fragment thereof described herein or the engineered cytokines described herein.

Provided herein are vectors including the polynucleotides encoding the engineered IL-21 cytokines or a functional fragment thereof described herein or the engineered cytokines described herein. Also provided herein are vectors, wherein the vectors are lentiviral vectors.

Also provided herein are host cells including the vectors disclosed herein. Also provided herein are host cells, wherein the host cells are immune cells. Also provided herein are host cells, wherein the immune cells are T cells or NK cells. Also provided herein are host cells, wherein the host cells further express a chimeric antigen receptor T cell.

Provided herein are oncolytic viruses including exogenous nucleic acids that code for the engineered IL-21 cytokines or a functional fragment thereof described herein or the engineered cytokines described herein. Also provided herein are oncolytic viruses, wherein the oncolytic viruses include lentiviruses.

### EXAMPLES

### Example 1: Generation of Interleukin (IL) Variants

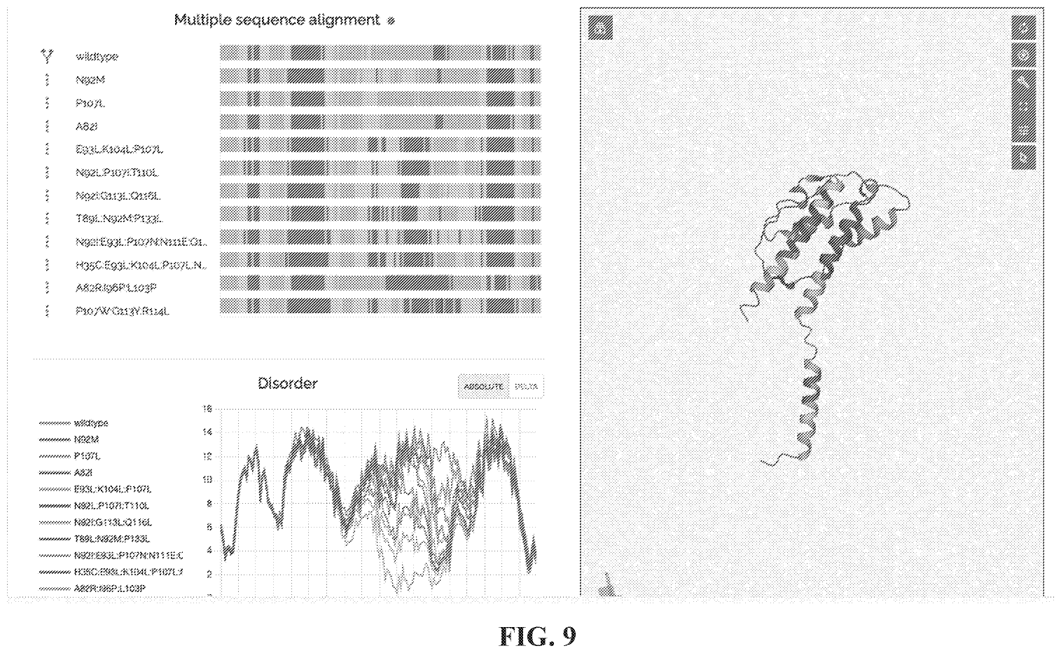

As illustrated in FIG. 1, a wild type sequence of an interleukin (e.g., an interleukin with an amino acid sequence as described in TABLE 1 or TABLE 2) was input into an AI software stack to generate initial AI model and its dynamics. Evolutionary covariance-based mapping of biological interfaces were performed to generate potency data, while structural disorder simulation was performed to generate stability and developability data for the AI-driven mutant design. Processes included structural ensemble modelling and reweighting of the experimental data as described in FIG. 9. The Fitness deciders include (1) the mean order propensity for each residue in the protein based on the existing data; (2) aggregation propensity; and (3) the likelihood per residue of what amino acid would be accepted. A list of positions not to be mutated was also provided, including the functional regions, post translational modifications, and regions likely to be cleaved (e.g., signaling peptides). The top candidates generated by the AI-driven mutant design were then tested in high-throughput experiments and data was used as experimental restraints for further optimizations.

The resulting interleukin variants resulting from an executed machine learning platform included sequences with mutations at one or more amino acid residues of the wild type sequence. The optimal resulting interleukin variants were then further screened for expression scaling and biological assay testing. The interleukin variant sequences correspond to interleukins with properties including an induced secondary structure and/or increased stability as compared to a wild type interleukin.

### Expression of Engineered Interleukins

To investigate expression and purification yields of the designed engineered interleukins, 50 mL cultures of HEK-293 kidney cells were transfected with the appropriate DNA following standard methods. The resulting cultures were harvested once the viability dropped below 90%. Culture supernatant was clarified by centrifugation at 4,000 G for 10 minutes and was then subsequently engineered by additions of: a 1× concentration protease inhibitor cocktail, 20 mM Tris pH 8.0, and 0.02% sodium azide before being frozen at −80 degrees Celsius.

### Immobilized Metal Ion Affinity Chromatography (IMAC) Purification

Culture supernatants previously frozen at −80 degrees Celsius as described above were thawed in a room temperature water bath then passed over 200 μL of Ni Excel resin in a spin column, using a 50 ml syringe as sample reservoir. The resin was then washed with 2×600 μl PBS, 2×600 μL PBS+20 mM imidazole, and then bound protein was eluted in 2×300 μL PBS+500 mM imidazole.

### SDS-Page Analysis

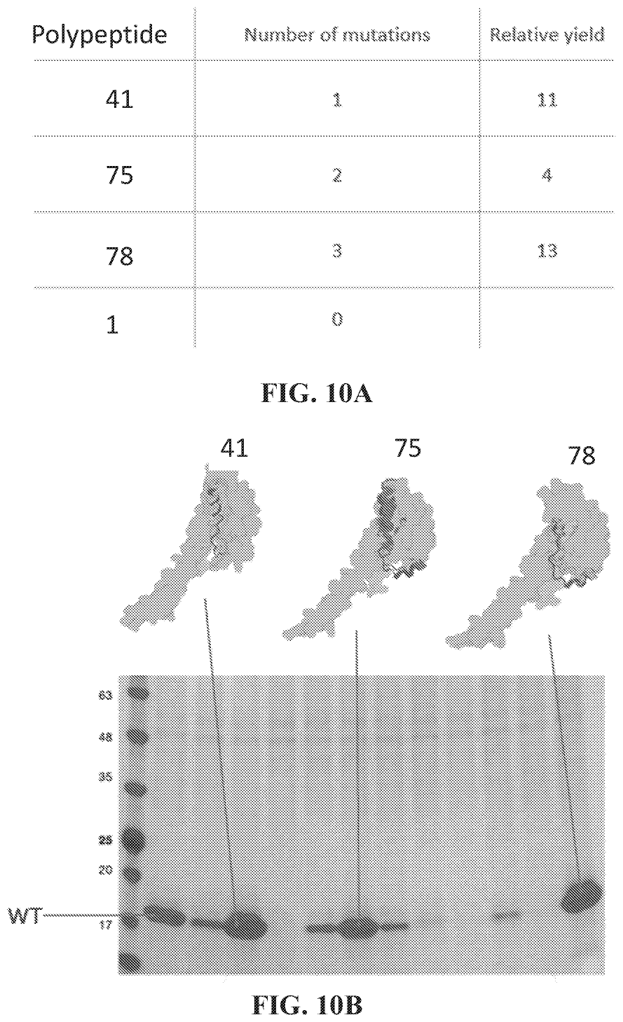

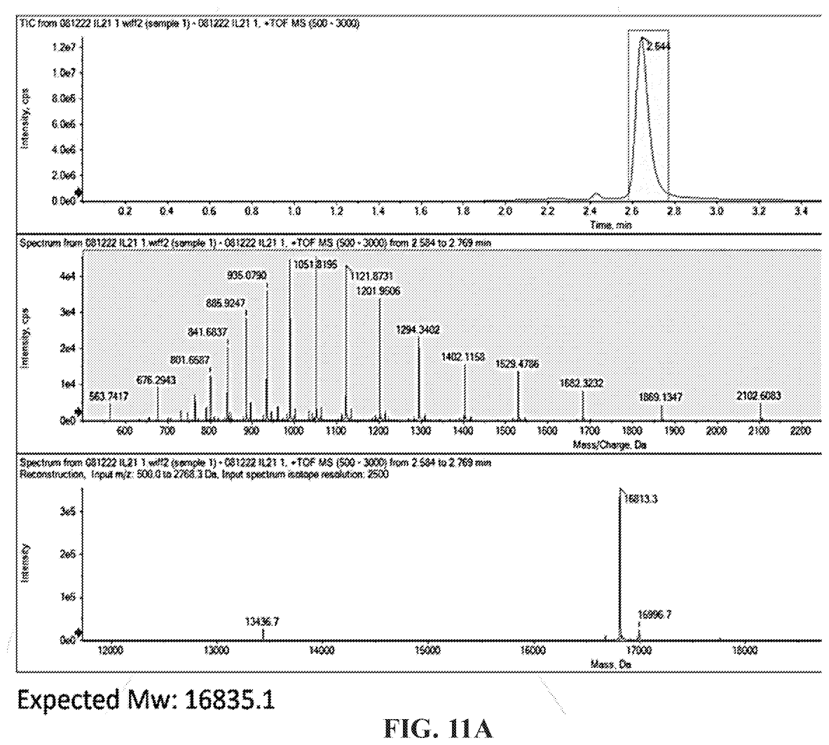

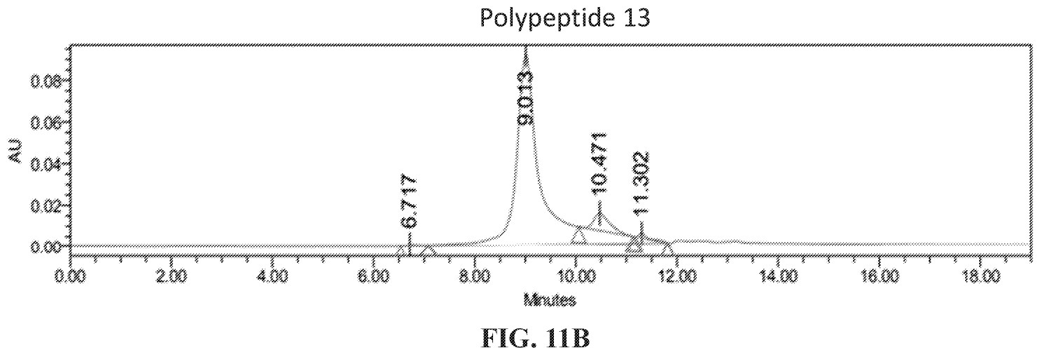

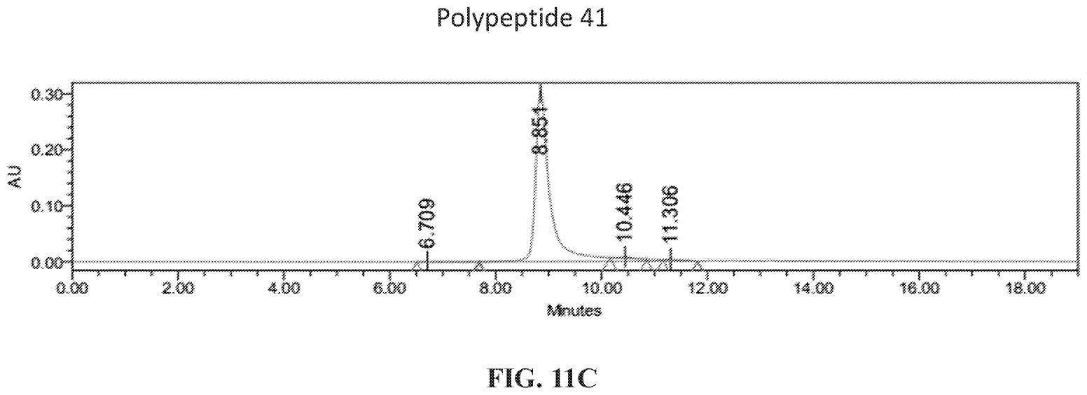

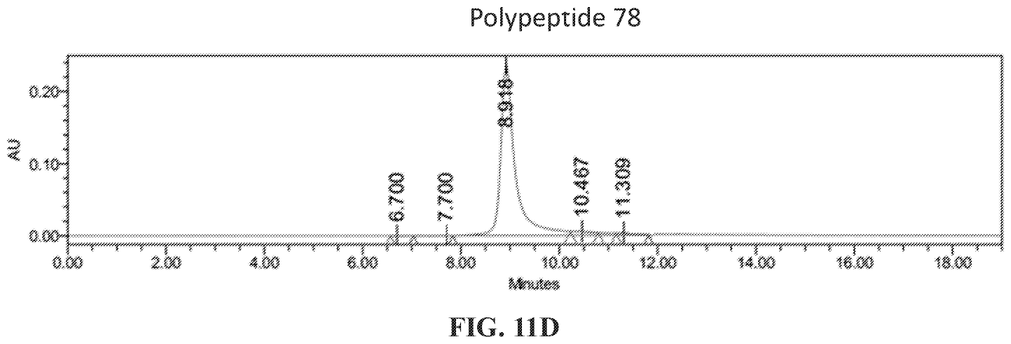

Eluted proteins purified as described above were diluted 1:1 with 2× concentration loading dye and boiled before 5 μL of each was loaded onto a 15-well gel (left), following standard protocols of SDS-PAGE analysis. The relative yield for Polypeptide 41, Polypeptide 75, and Polypeptide 78 as compared to the wild type (Polypeptide 1) are shown in FIG. 10A and the SDS-PAGE analysis is shown in FIG. 10B. The results indicate that Polypeptide 41, Polypeptide 75, and Polypeptide 78 are successfully expressed and purified with better yield as compared to the IL-21 wild-type (Polypeptide 1). The yield for Polypeptide 41, Polypeptide 75, and Polypeptide 78 are 11, 4, and 13 times as that of the IL-21 wild type. Based on these results and further sample processing, Polypeptide 41, Polypeptide 75, and Polypeptide 78 were chosen for an upscaled expression and purification. Proteins were expressed in 500 ml HEK293 cell cultures, following standard protocols. Supernatants were supplied with 0.02% sodium azide and 1× concentration Protease Inhibitors, pH was adjusted to 7.5 and samples were incubated overnight with 2 milliliters Ni Excel resin (prewashed in phosphate buffered saline (PBS). Samples were then subsequently passed through an econocolumn and washed with 10 milliliters PBS, 10 milliliters of a wash buffer (1×PBS pH 7.4:20 mM imidazole), and protein was eluted with 5× concentration at 2 milliliters volume of elution buffer (1×PBS pH 7.4:500 mM imidazole, passing the first elution over the resin twice). Fractions were pooled according to SDS-PAGE analysis and were concentrated to 2-3 milliliters using 10 kDa MWCO centricons, centrifuged to remove any precipitation (which, for some samples, was observed), and loaded on 16/60 S75 (“Lloyd”). Eluted at 1 ml/minute with SEC buffer (1×PBS pH 7.4), and collected as 2 milliliter fractions. Upscaled samples underwent quality control by using intact mass spectrometry (identity), SDS-PAGE (purity) and SEC (aggregation and purity). As shown in FIG. 11A-FIG. 11D, IL-21 WT and the variants (Polypeptide 41 and Polypeptide 78) show similar intact mass spectrometry profiles and SEC elution profiles.

### Analysis of Stability and Size

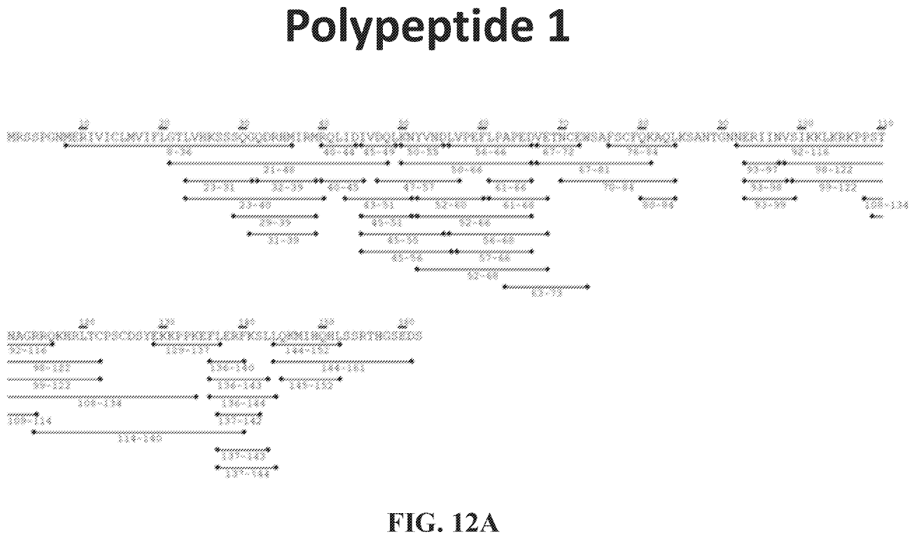

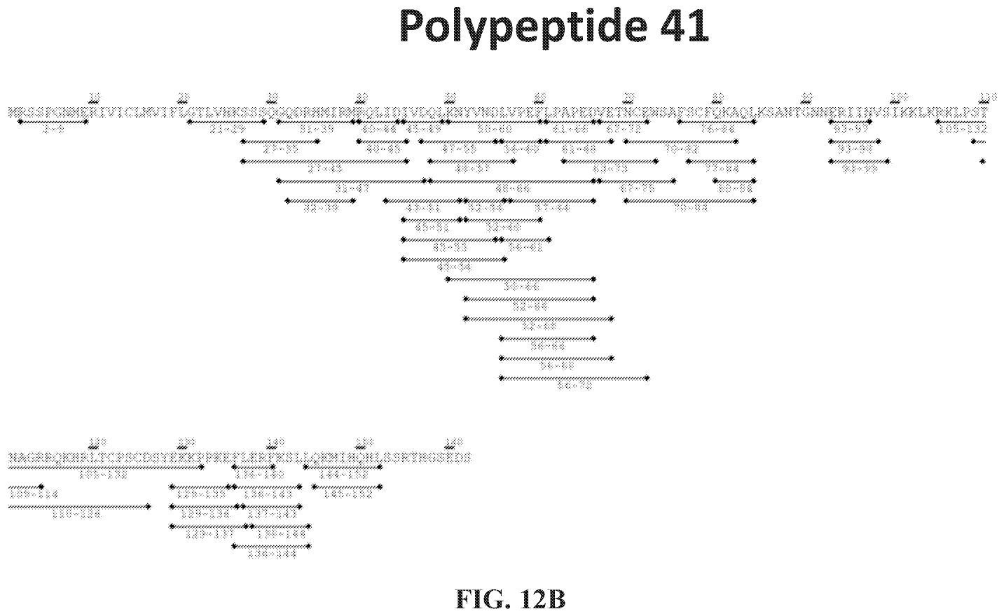

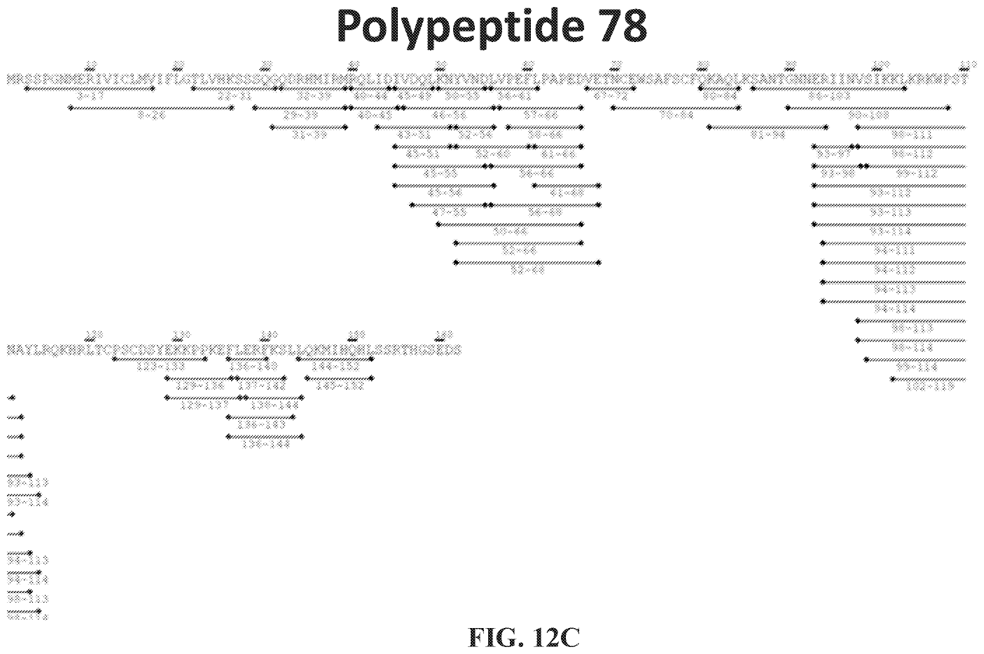

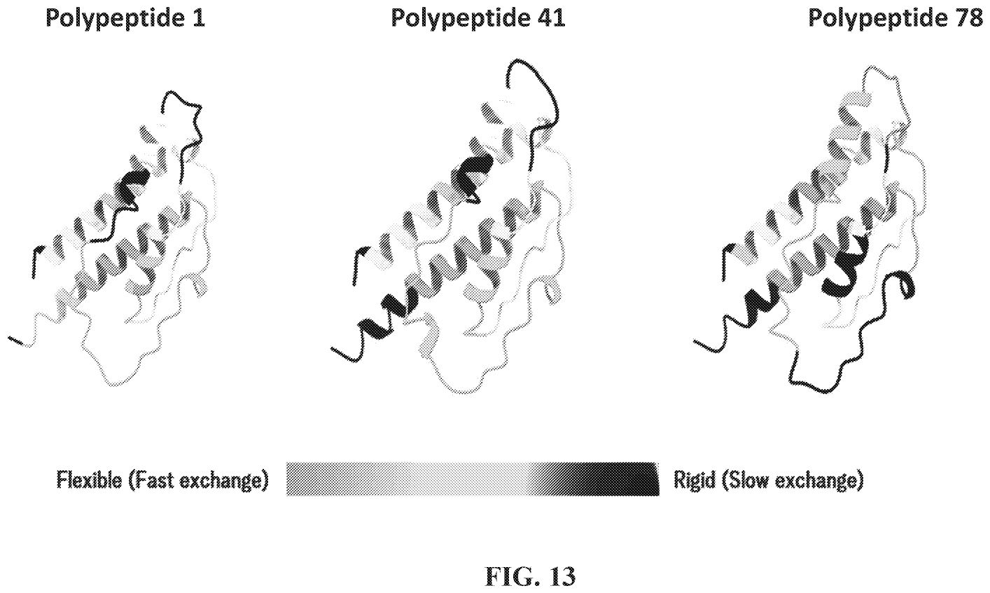

To evaluate the resistance of IL-21 variants to pepsin digest, IL-21 WT (Polypeptide 1) and variants (Polypeptide 41 and Polypeptide 78) are incubated with pepsin at 15 degrees Celsius for 5 min. The digested fragments are treated and labeled for MALDI-TOF analysis (FIG. 12A-FIG. 12C). Digestion was measured as a function of peptide coverage for Polypeptide 1, Polypeptide 41, and Polypeptide 78 resulting in 90.74%, 78,39%, and 70.11% digestion respectively. Polypeptide 41 and Polypeptide 78 show increased resistance to pepsin digestion as compared to that of the IL-21 WT (Polypeptide 1).

Sizing and thermal stability measurements with IL-21 variants were performed with a Prometheus Panta instrument (NanoTemper Technologies), based on a method setup experiment (data not shown) that identified optimal protein concentrations for the size measurements using DLS (dynamic light scattering) and thermal stability analyses using Tm (thermal melting) measurements. This approach allowed for the determination of low concentration Tm measurements to provide measurements as close as possible to the native Tm that is unbiased by aggregation effects. Protein concentrations for the analytical DLS measurements were chosen with the aim of obtaining a reasonable light scattering signal. Sample concentrations for the analytical assays for the DLS measurements were each 250 micrograms/milliliter, 75 micrograms/milliliter for the Tm measurements. Experimental sample dilutions to produce 250 ug/mL samples are shown in TABLE 5.

TABLE 5
Analytical sample preparations for interleukin
variant DLS measurements

| | Conc. | Target conc. | Target Vol. | PBS | Stock | |
|---|---|---|---|---|---|---|
| Polypeptide | (μg/mL) | (μg/mL) | (μL) | (μL) | (μL) | Use |
| 1 | 804.44 | 250 | 40 | 27.57 | 12.43 | DLS |
| 41 | 1223.65 | 250 | 40 | 31.83 | 8.17 | DLS |
| 78 | 968.65 | 250 | 40 | 29.68 | 10.32 | DLS |

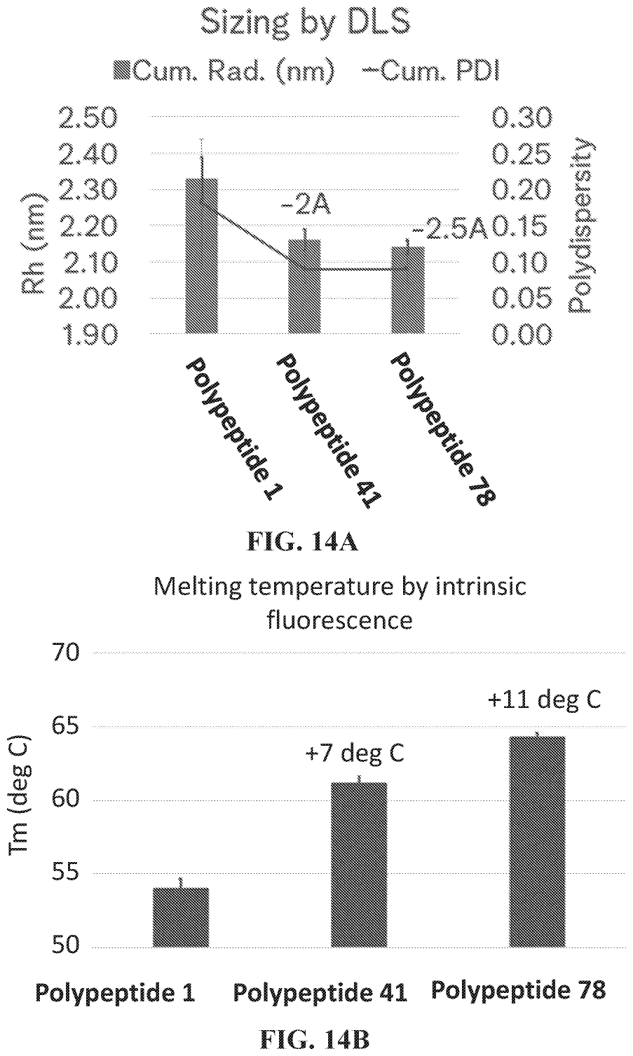

The samples were spun in a centrifuge for 30 minutes at 19,090 rpm at 4 degrees Celsius. All samples and a PBS blank sample were measured in triplicate by using high sensitivity capillaries. The DLS high sensitivity analysis was performed at 20 degrees Celsius, with maximal laser power. As shown in FIG. 14A, the size of the Polypeptide 41 is about 0.20 nm smaller than that of the IL-21 wild type. The size of the Polypeptide 78 is about 0.25 nm smaller than that of the IL-21 wild type. The reduced size of Polypeptide 41 and Polypeptide 78 indicates increased compactness.

The Tm of each sample was determined by performing a thermal ramp with a 1 degree Celsius/minute at a heating rate from 20 degrees Celsius to 95 degrees Celsius, using the maximal laser power and measuring the backscattered light as a proxy for turbidity to detect heat-induced aggregation. The Tm of each sample was detected automatically by the instrument software from the first derivative of the fluorescence ratio 330 mm/350 nm trace. Results were reported as mean±standard deviation from triplicate sample measurements. The turbidity traces consisting in the backscattered light intensity showed a flat trace below the threshold of 100 mAU throughout the entire thermal ramp indicating no aggregation in all samples at 75 ug/mL.

Experimental sample dilutions to produce 75 ug/mL are shown in TABLE 6 and the results for the Tm detections are shown in FIG. 14B. As shown in FIG. 14B, the Tm of Polypeptide 41 and Polypeptide 78 increased by about 7 degrees Celsius and 11 degrees Celsius, respectively, as compared to that of the IL-21 wild type.

TABLE 6
Analytical sample preparations for
interleukin variant Tm measurements

| | Conc. | Target conc. | Target Vol. | PBS | Stock | |
|---|---|---|---|---|---|---|
| ID | (ug/mL) | (ug/mL) | (uL) | (uL) | (uL) | Use |
| 1 | 250 | 75 | 30 | 21 | 9 | Tm |
| 41 | 250 | 75 | 30 | 21 | 9 | Tm |
| 78 | 250 | 75 | 30 | 21 | 9 | Tm |

### Example 2: Bioassay Design and Procedures of IL-21 Variants

To evaluate biological activity of IL-21 wild type and variants thereof, an MC116 cell line was selected. The MC116 cell line is a diffuse large B-cell Lymphoma (DLBCL) isolated from the ascites of a lymphoma patient. Aspects evaluated relating to interleukin variant function included: (1) IL-21R expression on MC116 cell lines (as shown in Example 3): (2) the viability of MC116 cells after IL-21 wild type and variant treatments (as shown in Example 4): (3) the induction of STAT3 phosphorylation by IL-21 wild type and variant treatments in MC116 cells (as shown in Example 5); and (4) the IL-21 wild type/variant apoptosis induction in MC116 cells (as shown in Example 6).

Cells were grown in RPMI medium supplemented with 20% h.i.FBS and 1% of Penicillin-Streptomycin. Cultures were maintained in a humidified incubator at 37 degrees Celsius in 5% CO₂. The medium used in the experiments steps were as follows: (i) the pre-seeding step was performed in RPMI 20% h.i.FBS 1% PS; (ii) the starvation step was performed in RPMI 5% h.i.FBS 1% PS; (iii) the IL-21 wild type and variants treatments to trigger STAT3 phosphorylation were performed in RPMI 5% h.i.FBS 1% PS; and (iv) the IL-21 wild type and variants treatments during cell viability and apoptosis induction assays were performed in RPMI 10% h.i.FBS 1% PS.

### Example 3: Receptor Expression Analyses

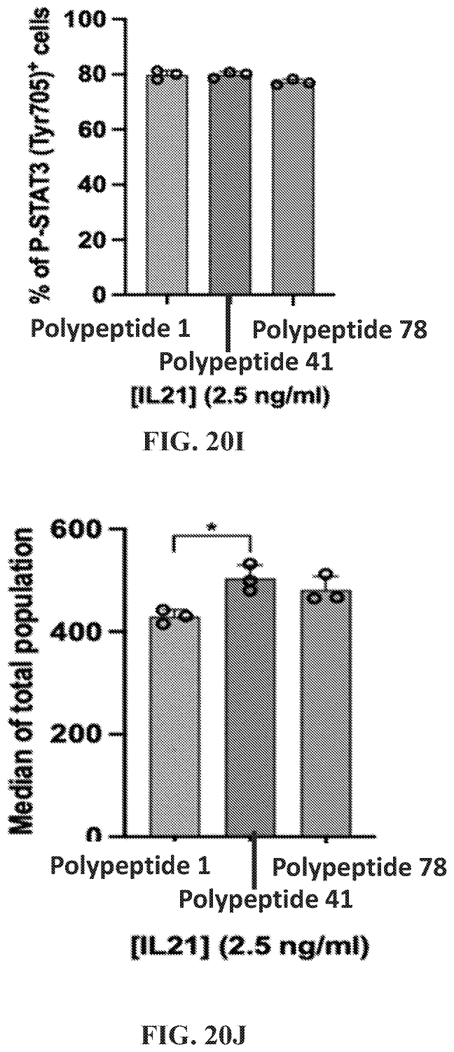

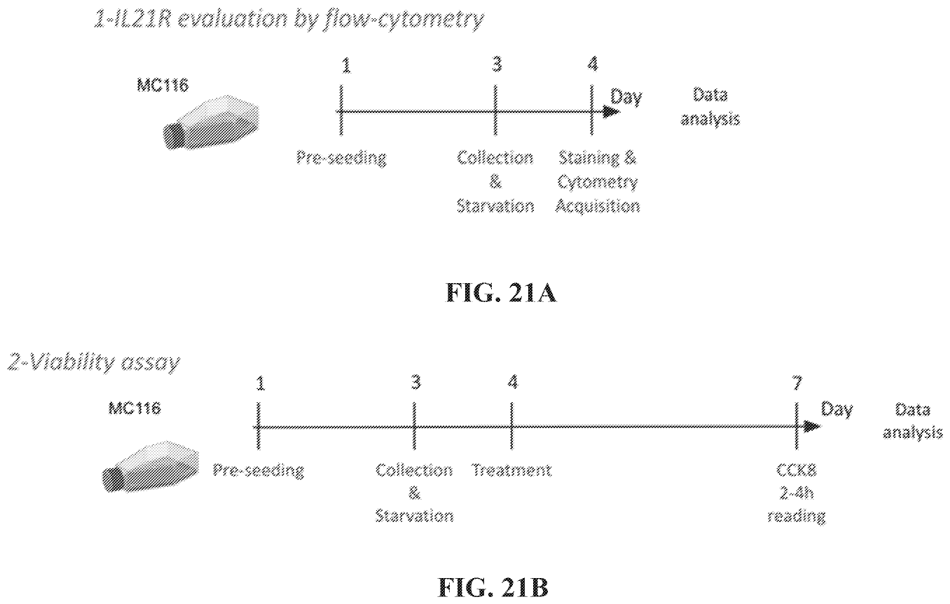

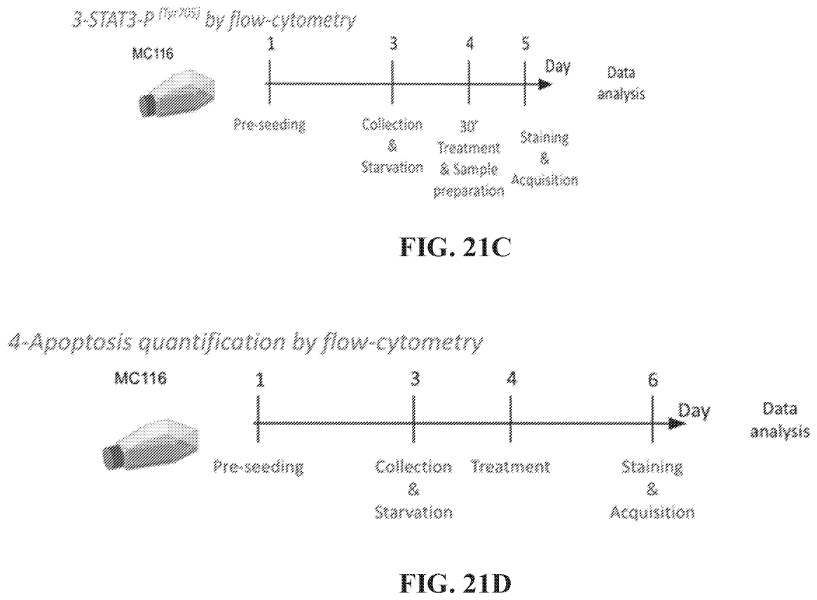

To demonstrate that the IL-21 receptor is present on the MC116 cell surface, cells were analyzed by flow cytometry as described in FIG. 21A. At day 1, MC116 cells were pre-seeded at a density of 0.1×10⁶/m in RPMI 20% h.i.FBS. After 48 hours, the cells were collected and centrifuged at 150 G for 5 minutes and starved in RPMI 5% h.i.FBS at a density of 0.4×10⁶/ml overnight. After starvation cells were collected and stained to assess the IL-21Ra chain (CD360) expression.

Harvested cells were washed with 2 ml of FACS Buffer (5-10% FBS) by centrifugation at 1500 rpm for 5 seconds. The supernatant was then discarded. 2-2.5 ug of Human BD Fc Block™ (BD 564219) were added to each tube to avoid non-specific binding (background fluorescence) and incubated for 10 minutes at room temperature. Conjugated mAbs were added and incubated for 15-20 minute at 4 degrees Celsius in the dark. The specific antibody used was a PE anti-human CD360 (IL-21R) Antibody-CLONE 17A2-Biologend 651004 #Lot B335052; Isotype control: PE Mouse IgG1, κ Isotype Control-Biolegend 400114 #Lot B362215. Cells were washed 2 times with FACS Buffer by centrifugation at 1500 rpm for 5 minutes and resuspended in 200-500 μl of ice cold FACS buffer. Before acquiring 7AAD (BD 559925) was added to exclude dead cells, and then the cells were acquired by BD FACSMelody™ Cell Sorter.

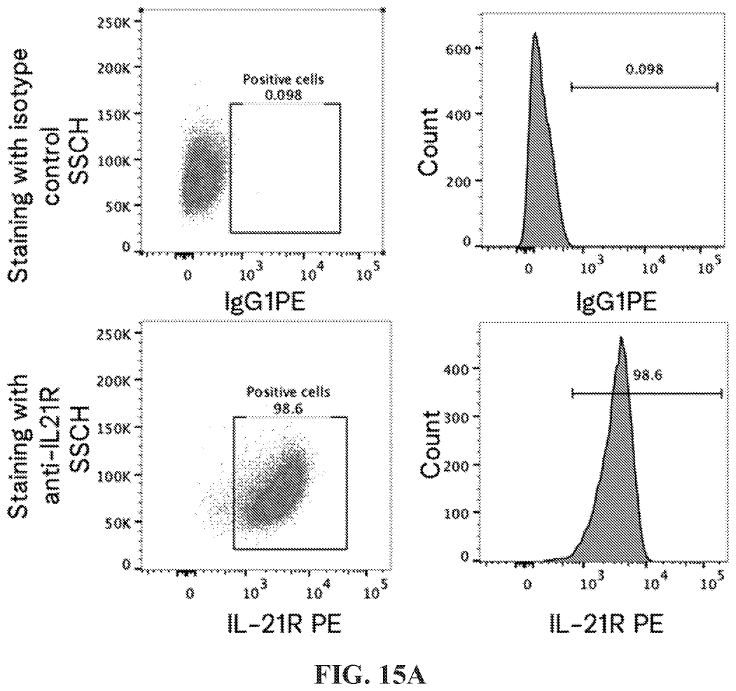

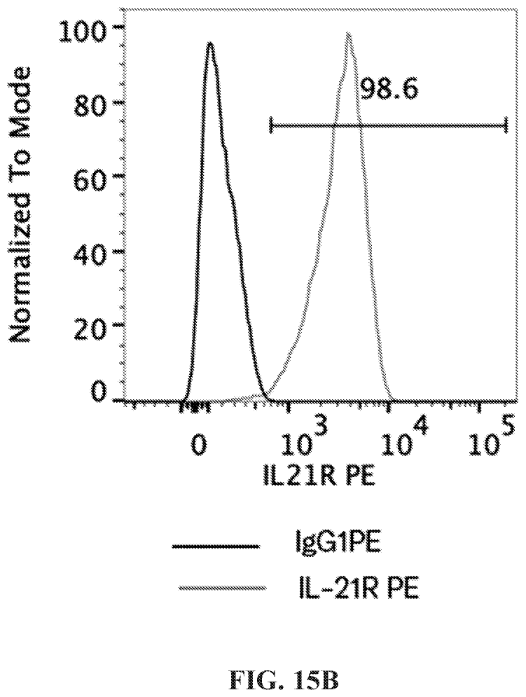

As shown in FIG. 15A-FIG. 15B, positive cells were not detected when the cells were stained with isotype control, but are shown when cells are stained with the anti-IL-21R Antibody. The results indicate IL-21R expression in MC116 cell lines.

### Example 4: Cell Viability Measurements

To investigate the impact of IL-21 variants on MC116 cell viability, the cells were treated as described in FIG. 21B. At day 1 MC116 cells were pre-seeded at a density of 0.1×106/m in RPMI 20% h.i.FBS. After 48 hours cells were collected and centrifuged at 150 g for 5 minutes and starved in RPMI 5% h.i.FBS at a density of 0.4×10 c/ml overnight. 0.04×10⁶ cells/well were plated in 96 flat well plate. After the starvation, pre-diluted stimuli were added at different concentrations. Final concentrations from 100 ng/ml to 6.25 ng/ml in medium with 10% of h.i. FBS (final concentration). Cells were incubated for 72 hours in a humidified incubator (at 37 degrees Celsius, 5% CO₂). After 72 hours, to determine cell viability a standard curve was prepared by using MC116 cells and CCK-8 kit. Accordingly with the instruction 10 μl of the CCK-8 solution were added to each well of the plate. The plate was incubated in a humidified incubator (at 37 degrees Celsius, 5% CO₂) for 3 hours and then the absorbance was measured at 450 nm using a microplate reader.

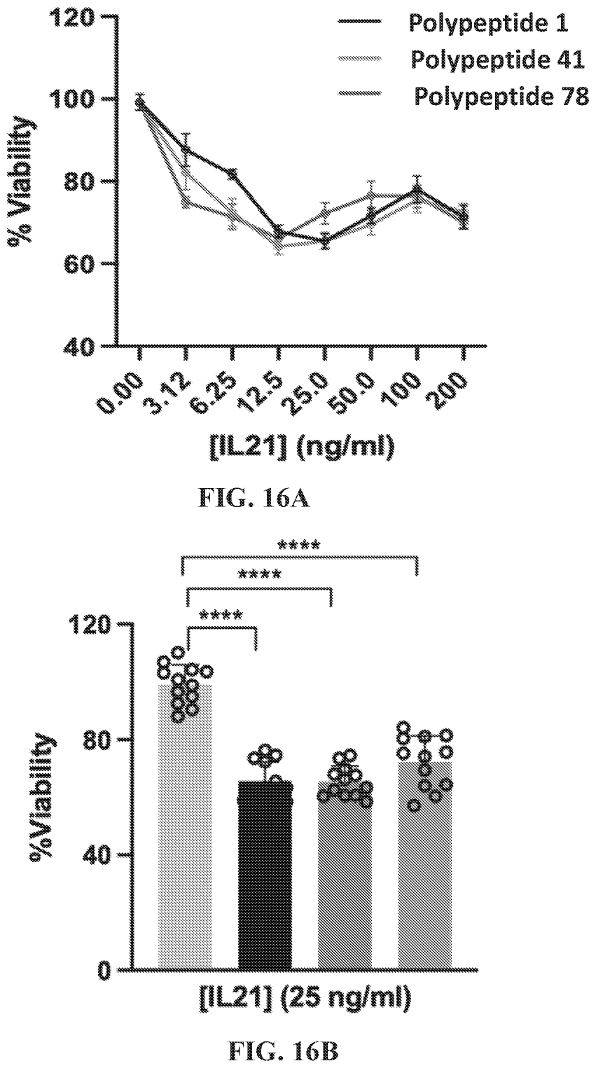

As shown in FIG. 16A, MC116 cell viability slightly decreased from 100% to 60% when the concentration of IL-21 wild type or variants increased to 12.5 ng/ml, but then fluctuated between 60% and 80% when the concentration of IL-21 wild type or variants was higher than 12.6 ng/ml. at 25 ng/ml, MC116 cell viability for IL-21 wild type, Polypeptide 41, and Polypeptide 78 are comparable to each other, but all are lower than that in the absence of any IL-21 (FIG. 16B). Therefore, Polypeptide 41 and Polypeptide 78 demonstrate % viabilities of MC116 cells comparable to IL-21 viability.

### Example 5: STAT3 Phosphorylation

To investigate the impact of IL-21 variants on STAT3 phosphorylation, MC116 cells were treated as described in FIG. 21C. MC116 cells were pre-seeded at 0.1×10⁶/m in RPMI 20% h.i.FBS, 48 hours later cells were collected and centrifuged at 150 g for 5 minutes and starved in RPMI 5% h.i.FBS at 0.4×106/ml overnight. After the starvation cells were collected and centrifuged at 150 g for 5 minutes. 0.3×10⁶ cells/tube were suspended in RPMI 5% h.i.FBS at 1×10⁶/ml and stimuli were added and incubated for 30 minutes at 37 degrees Celsius. Then cells were stained as is described below. Cells were fixed immediately after treatment by adding an equal volume of pre-warmed Fixation Buffer (Biolegend 420801), and they were incubated at 37 degrees Celsius for 15 minutes. Cells were centrifuged at 350×g at room temperature for 5 minutes, supernatant was discarded and cell pellet was resuspended by vortexing. Cells were washed by adding 2 ml of FACS Buffer (5-10% FBS) and centrifuged at 350×g at room temperature for 5 minutes for a total of two washes. Cell pellet was resuspended by gently pipetting using the residual volume. While vortexing, cells were permeabilized by adding pre-chilled True-Phos™ Perm Buffer (Biolegend 425401). 0.3×10⁶ cells were permeabilized with 0.3 ml of pre-chilled True-Phos™ Perm Buffer. Samples were stored overnight at −20 degrees Celsius. Samples were centrifuged at 1000×g at room temperature for 5 minutes, decant supernatant was discarded, and cell pellets were resuspended by vortexing. Cells were washed with 2 mL of FACS Buffer (5-10% FBS) and centrifuged at 1000×g at room temperature for 5 minutes, for a total of two times. Cells were resuspended in 100 μL of FACS Buffer (5-10% FBS) and conjugated mAb were added and incubated for 30 minutes at room temperature in the dark. Cells were washed with 2 ml of FACS Buffer (5-10% FBS) and centrifuged at 1000×g at room temperature for 5 minutes, for a total of two times. Conjugated mAb was added and incubated for 15-20 min at 4 degrees Celsius in the dark (PE anti-STAT3 Phospho (Tyt705) Biolegend 651004 #Lot B341628). After discarding the supernatant cells were resuspended in 300 μL of FACS Buffer and acquired by BD FACSMelody™ Cell Sorter.

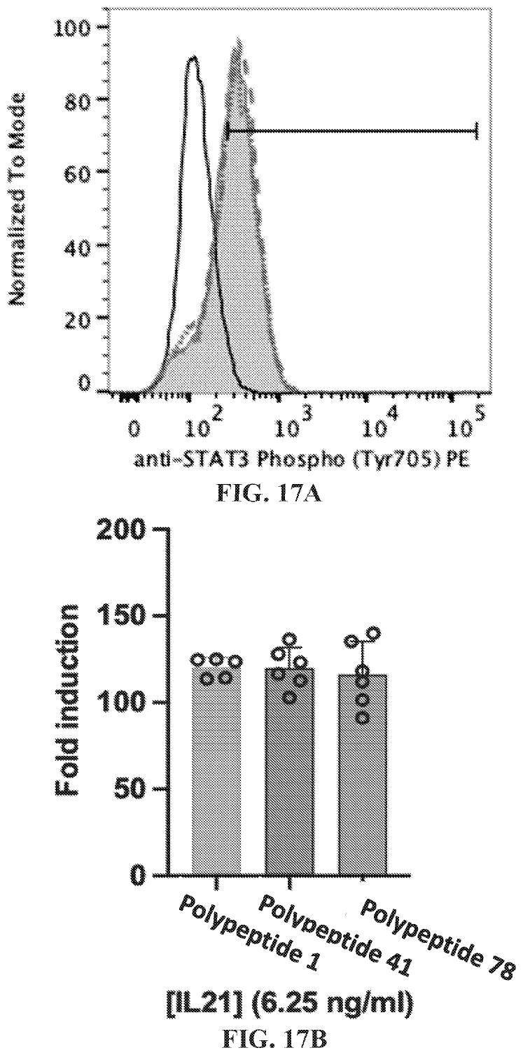

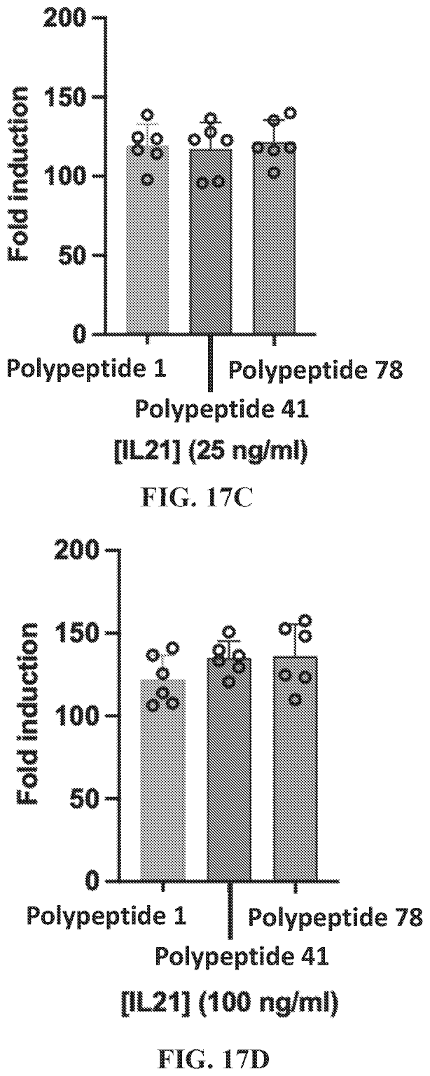

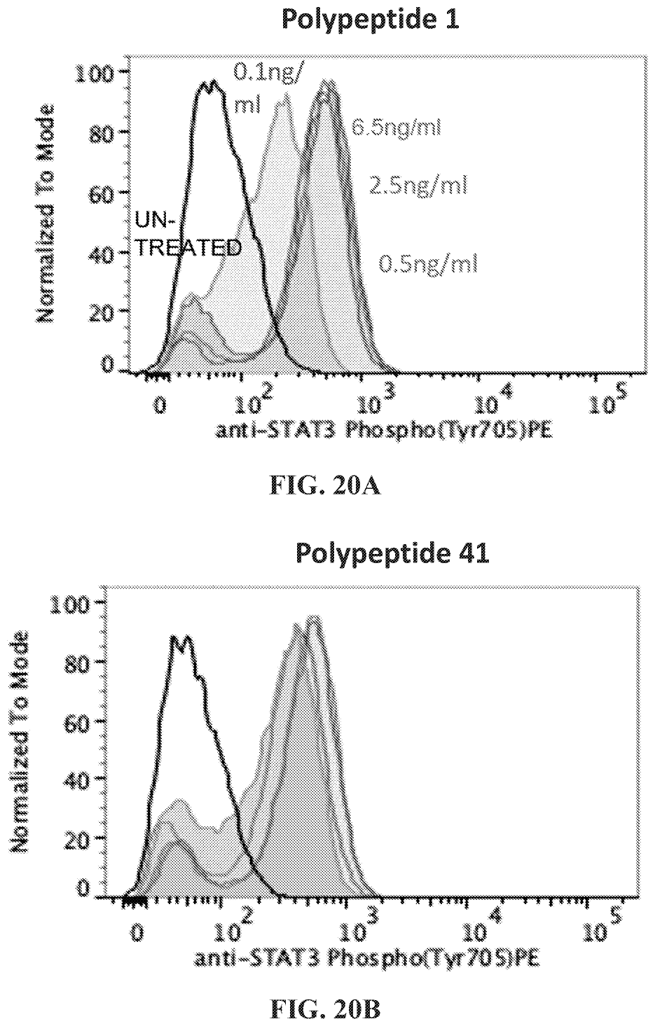

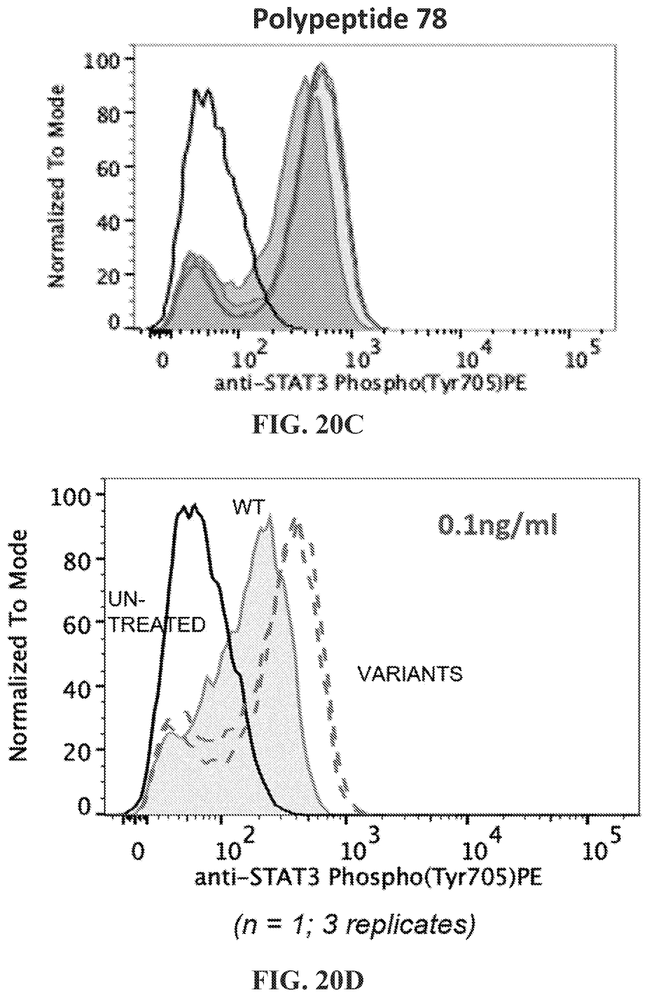

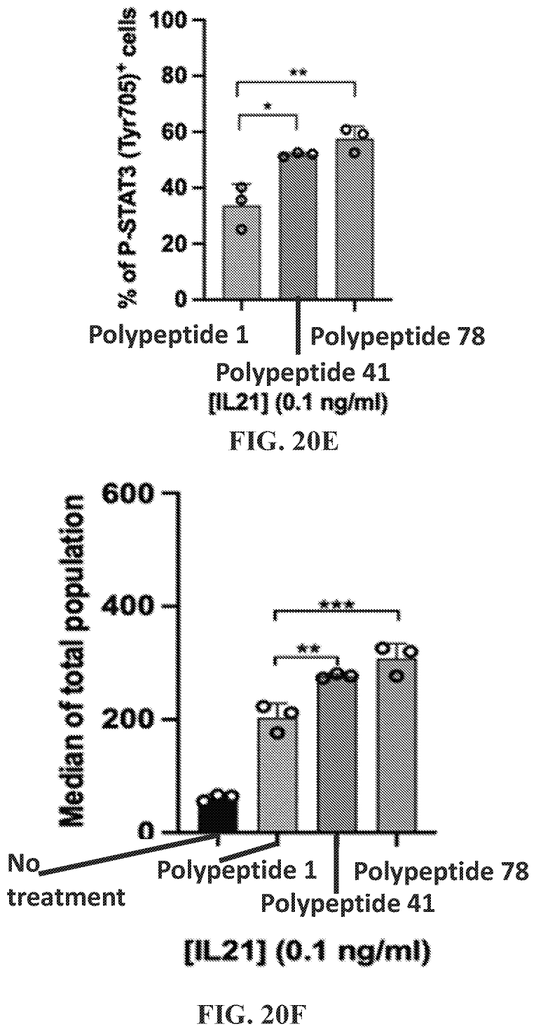

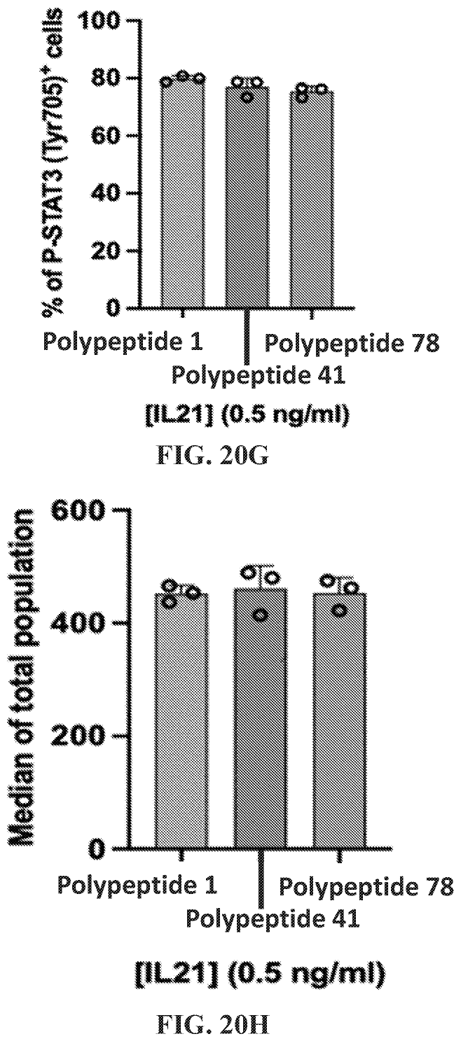

As shown in FIG. 17A-FIG. 17B and FIG. 20A-FIG. 20J, STAT3 phosphorylation is induced by both IL-21 wild type (polypeptide 1) and variants (Polypeptide 41, and Polypeptide 78). The fold induction for the IL-21 variants (Polypeptide 41, and Polypeptide 78) at 6.25 ng/ml is comparable to that of IL-21 wild type (Polypeptide 1) (FIG. 17B). The results show that at low concentration ranges, such as 0.1 ng/ml, Polypeptide 41 and Polypeptide 78 induces higher STAT3 phosphorylation (FIG. 20E-FIG. 20F) as compared to that of IL-21 wild type.

### Example 6: Measurements of Apoptosis

To investigate the impact of IL-21 variants on apoptosis MC116 cells were treated as described in FIG. 21D. MC116 cells were pre-seeded at a density of 0.1×106/m in RPMI 20% h.i.FBS. After 48 hours cells were collected and centrifuged at 150 g for 5 minutes and starved in RPMI 5% h.i.FBS at a density of 0.4×106/ml overnight. 0.04×10⁶ cells/well were plated in a 96 flat well plate. After the starvation, pre-diluted stimuli were added at different concentrations. Final concentrations: 100 ng/ml, 50 ng/ml, 25 ng/ml and 6.25 ng/ml in medium with 10% of h.i. FBS (final concentration). Cells were incubated for 48 h in a humidified incubator (at 37 degrees Celsius, 5% CO₂). After 72 hours cells were collected and the apoptosis was detected as is described below. The cells were washed by adding 2 ml of cold PBS and centrifuge at 1500 rpm for 5 minutes. After discarding the supernatant, cells were resuspended in Annexin V Binding Buffer at a concentration of 0.25-1.0×107 cells/ml. 5 μL of FITC Annexin V and 5 μL of 7-AAD Viability Staining Solution were added. Cells were gently vortexed and incubated for 15 minutes at room temperature in the dark. 200 μL of Annexin V Binding Buffer were added to each tube and sample were acquired by BD FACSMelody™ Cell Sorter.

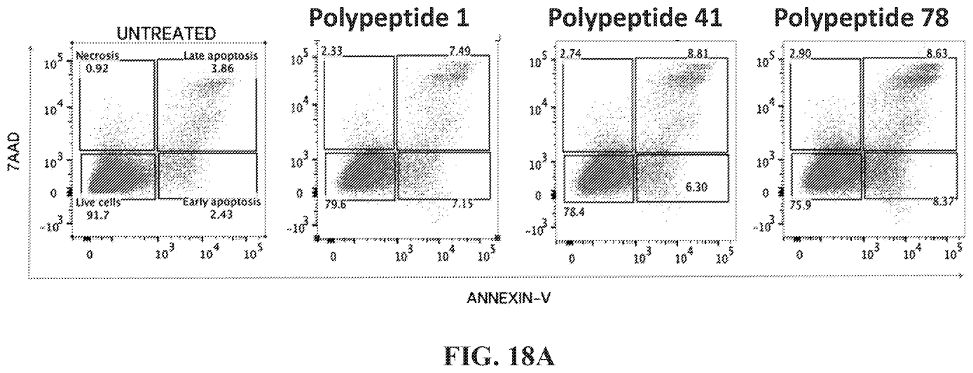

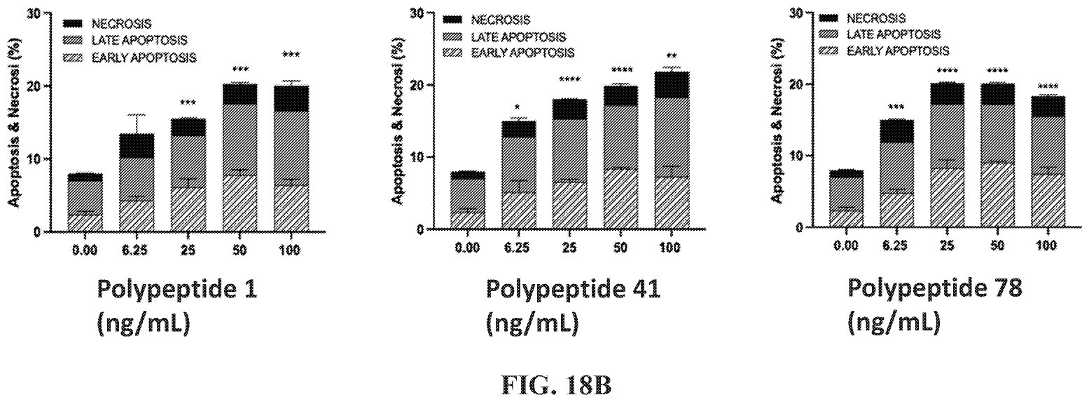

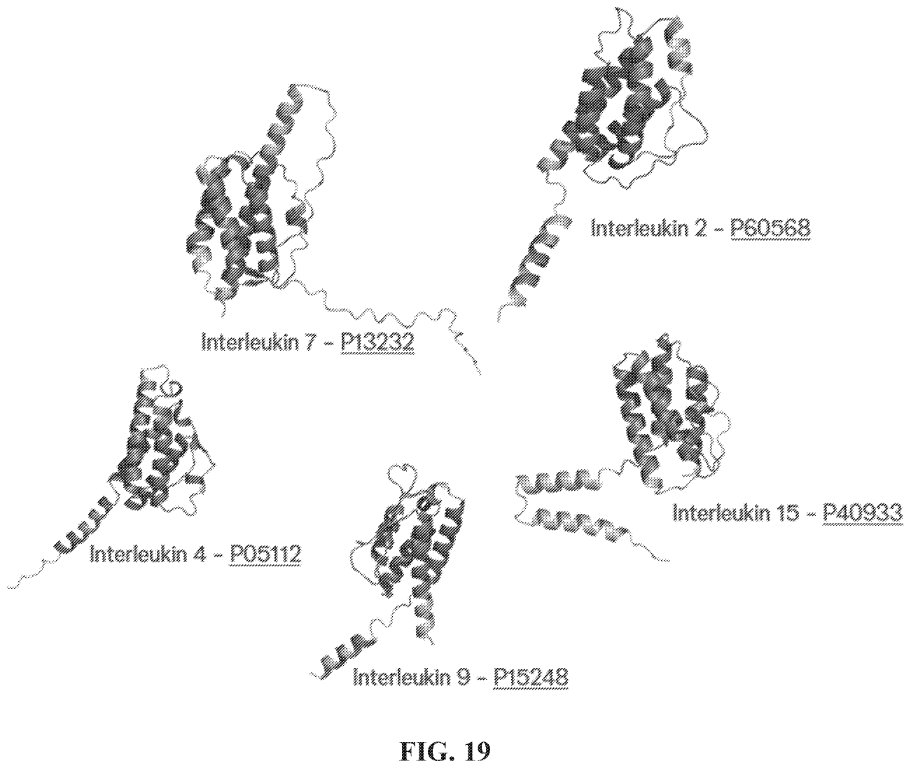

As shown in FIG. 18A-FIG. 18B, the percentage of apoptosis and necrosis increased in the presence of increasing concentration of IL-21 W wild type and variants (Polypeptide 41 and Polypeptide 78) from 0 to 50 ng/ml. at concentrations higher than 50 ng/ml, Polypeptide 1 and Polypeptide 78 do not induce more apoptosis and necrosis, while Polypeptide 41 still induces more apoptosis and necrosis. Therefore, the IL-21 variants (Polypeptide 41 and Polypeptide 78) induce MC116 cell death activity similar to wild type.

### Example 7: Generation of Cytokine Variants

A wild type sequence of an cytokine is input into an AI software stack to generate initial AI model and its dynamics. Evolutionary covariance-based mapping of biological interfaces is performed to generate potency data, while structural disorder simulation is performed to generate stability and developability data for the AI-driven mutant design. The Fitness deciders include (1) the mean order propensity for each residue in the protein based on the existing data: (2) aggregation propensity; and (3) the likelihood per residue of what amino acid would be accepted. A list of positions not to be mutated is also provided, including the functional regions, post translational modifications, and regions likely to be cleaved (e.g., signaling peptides). The top candidates generated by the AI-driven mutant design are then tested in high-throughput experiments and data was used as experimental restraints for further optimizations.

The resulting cytokine variants resulting from an executed machine learning platform include sequences with mutations at one or more amino acid residues of the wild type sequence. The optimal resulting interleukin variants are then further screened for expression scaling and biological assay testing. The cytokine variant sequences correspond to cytokines with properties including an induced secondary structure and/or increased stability as compared to a wild type cytokine.

### Example 8: Serum Stability

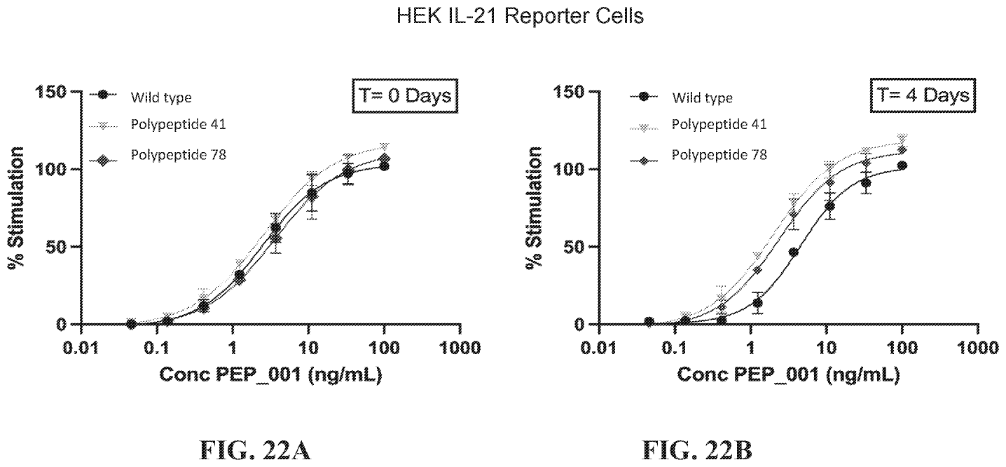

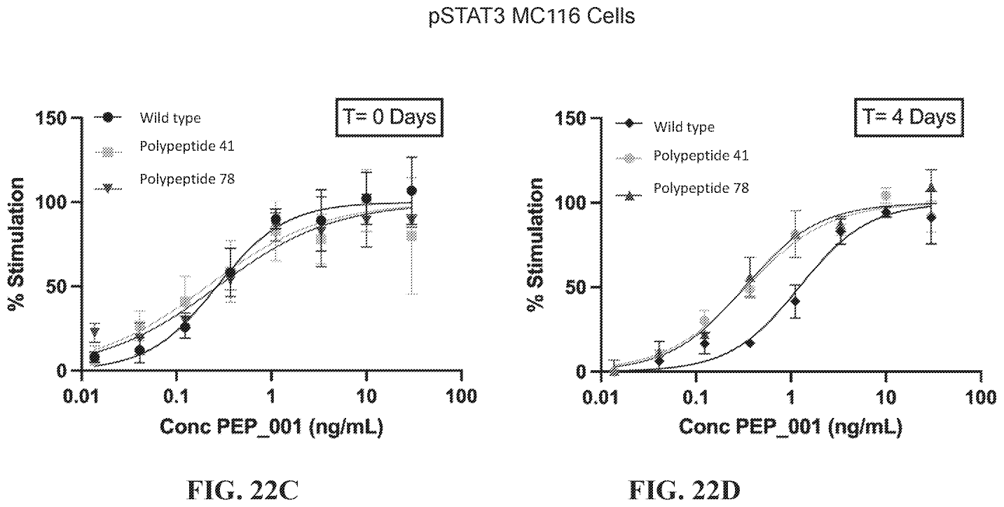

As shown in FIG. 22A and FIG. 22B, exemplary cytokine variants (Polypeptides 41 and Polypeptide 78) showed an enhanced in vitro serum stability. IL-21 function, as measured by HEK IL-21 reporter cells and STAT3 phosphorylation in physiologically relevant MC116 cells, was maintained over 4 days in a serum stability assay; exhibiting high binding resulting from an executed machine learning platform include sequences with mutations as described herein.

### Example 9: PK Profile

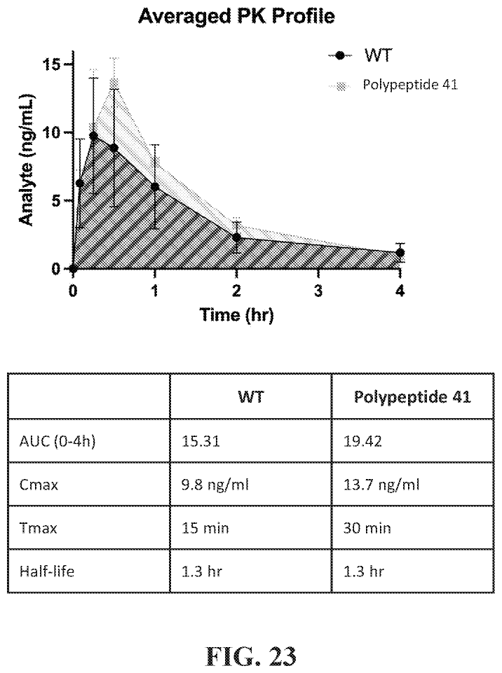

As shown in FIG. 23, an exemplary IL-21 variant (Polypeptide 41) showed improved an pharmacokinetic profile compared to wild type IL-21, with an improved area under the curve (AUC) as well as a higher Cmax and Tmax.

1.-180. (canceled)

181. A composition comprising:
an engineered IL-21 cytokine or a functional fragment thereof, that is a variant of a wild type IL-21 cytokine having amino acid residues of SEQ ID NO: 1,
wherein the engineered IL-21 cytokine or a functional fragment thereof comprises at least one amino acid substitution in a region comprising amino acid residues 30 to 135 of SEQ ID NO: 1,
wherein the at least one amino acid substitution in a region comprising amino acid residues 30-135 of SEQ ID NO: 1 is at position R34 or P107,
wherein the at least one amino acid substitution provides for an increased stability of the engineered IL-21 cytokine compared to the wild type IL-21 cytokine, and
wherein the increased stability is characterized by an increased thermal stability of the engineered IL-21 cytokine compared to that of the wild type IL-21 cytokine when measured in a 75 ug/mL sample using a differential scanning fluorimetry (DSF) spectroscopic method to detect thermal melting measurements by performing a thermal ramp with a 1 degree Celsius/minute heating rate from 20 degrees Celsius to 95 degrees Celsius and measuring backscattered light intensity to detect heat induced aggregation.

182. The composition of claim 181, wherein the DSF spectroscopic method determines a melting temperature of the engineered IL-21 cytokine or the wild type IL-21 cytokine, wherein the melting temperature of the engineered IL-21 cytokine is increased by at least 3 degrees Celsius as compared to that of the wild type IL-21 cytokine.

183. The composition of claim 181, wherein the increased stability is further characterized by an increased resistance to pepsin digestion of the engineered IL-21 cytokine compared to that of the wild type IL-21 cytokine, wherein the pepsin digestion produces peptide fragments, and wherein the increased resistance to pepsin digestion is evaluated by quantifying the peptide fragments measured by mass spectrometry.

184. The composition of claim 183, wherein the increased resistance is at least about 10%.

185. The composition of claim 181, wherein the increased stability is further characterized by an increased structural rigidity of the engineered IL-21 cytokine compared to the wild type IL-21 cytokine.

186. The composition of claim 185, wherein the increased structural rigidity is measured by an increased proton-deuterium exchange rate of the engineered IL-21 cytokine compared to that of the wild type IL-21 cytokine when measured by hydrogen deuterium exchange mass spectrometry (HDX-MS).

187. The composition of claim 181, wherein the increased stability is further characterized by an increased compactness of the engineered IL-21 cytokine compared to that of the wild type IL-21 cytokine as measured by dynamic light scattering (DLS).

188. The composition of claim 187, wherein the DLS determines an average particle diameter of the engineered IL-21 cytokine or the wild type IL-21 cytokine, wherein the average particle diameter of the engineered IL-21 cytokine is decreased by at least about 0.10 nm compared to that of the wild type IL-21 cytokine.

189. The composition of claim 181, wherein the increased stability is further characterized by an increased yield in an expression system of the engineered IL-21 cytokine compared to that of the wild type IL-21 cytokine.

190. The composition of claim 181, wherein the engineered IL-21 cytokine comprises the amino acid substitution at position P107 of SEQ ID NO: 1, and wherein the at least one amino acid substitution of SEQ ID NO: 1 at position P107 is P107L, P107I, P107A, P107P, P107Y, P107W, P107M, or P107N.

191. The composition of claim 190, wherein the at least one amino acid substitution of SEQ ID NO: 1 at position P107 is P107L.

192. The composition of claim 191, wherein the engineered IL-21 cytokine or a functional variant thereof comprises an amino acid sequence of SEQ ID NO: 41.

193. The composition of claim 181, wherein the engineered IL-21 cytokine or a functional variant thereof further comprises one amino acid substitution, two amino acid substitutions, three substitutions, or four amino acid substitutions OF SEQ ID NO: 1 selected from the group consisting of: H35, 137, R38, D44, 145, Q48, N70, A82, T89, G90, N92, E93, I95, I96, V98, K102, L103, K104, R105, T110, N111, A112, G113, R114, R115, Q116, H118, R119, L120, and P133.

194. The composition of claim 193, wherein the engineered IL-21 cytokine or a functional variant thereof further comprises one amino acid substitution with respect to SEQ ID NO: 1 at position G113.

195. The composition of claim 194, wherein the one amino acid substitution of SEQ ID NO: 1 at position G113 is G113L, G113I, G113A, G113P, G113Y, G113W, G113M, or G113Q.

196. The composition of claim 195, wherein the one amino acid substitution of SEQ ID NO: 1 at position G113 is G113Y.

197. The composition of claim 193, wherein the engineered IL-21 cytokine or a functional variant thereof further comprises one amino acid substitution of SEQ ID NO: 1 at position R114.

198. The composition of claim 197, wherein the one amino acid substitution of SEQ ID NO: 1 at position R114 is R114L, R114I, R114A, R114P, R114Y, R114W, or R114M.

199. The composition of claim 198, wherein the one amino acid substitution of SEQ ID NO: 1 at position R114 is R114L.

200. The composition of claim 193, wherein the engineered IL-21 cytokine or a functional variant thereof comprises at least three amino acid substitutions with respect to SEQ ID NO: 1.

201. The composition of claim 200, wherein the at least three amino acid substitutions 5 of SEQ ID NO: 1 are at positions P107, G113, and R114.

202. The composition of claim 201, wherein the at least three amino acid substitutions with respect to SEQ ID NO: 1 at positions P107, G113, and R114 are P107W, G113Y, and R114L.

203. The composition of claim 201, wherein the engineered IL-21 cytokine or a functional variant thereof comprises an amino acid sequence of SEQ ID NO: 78.
